(19) United States
(12) Patent Application Publication
(10) Pub. No.: US 2024/0307501 A1
(43) Pub. Date: Sep. 19, 2024

(54) TREATMENT OF CONGENITAL HYPERINSULINISM WITH AVEXITIDE

(71) Applicant: Eiger Biopharmaceuticals, Inc., Palo Alto, CA (US)

(72) Inventor: Colleen M. Craig, Palo Alto, CA (US)

(73) Assignee: Eiger Biopharmaceuticals, Inc., Palo Alto, CA (US)

(21) Appl. No.: 18/572,074

(22) PCT Filed: Jun. 21, 2022
(86) PCT No.: PCT/US2022/034415

§ 371 Date: Dec. 19, 2023

Related U.S. Application Data
(60) Provisional application No. 63/213,051, filed on Jun. 21, 2021.

(51) Int. Cl. A61K 38/26; A61P 3/08

(52) U.S. Cl. CPC A61K 38/26; A61P 3/08

(57) ABSTRACT

Methods of treating congenital hyperinsulinism (CHI) are provided. In particular, the present disclosure provides for treatment of CHI by administering avexitide to a subject having CHI. The provided methods can involve subcutaneously administering avexitide to a subject having CHI. In some instances, a reduction in the glucose infusion rate (GIR) necessary to maintain euglycemia in the subject is achieved by administering avexitide to the subject. In some instances, administering avexitide reduces the hypoglycemia event rate in a subject having CHI.

### CROSS-REFERENCE TO RELATED APPLICATIONS

This application claims the of and priority to U.S. Provisional Application No. 63/213,051, filed Jun. 21, 2021, the contents of which are incorporated by reference herein.

### SEQUENCE LISTING

The official copy of the sequence listing is submitted electronically via EFS-Web as an ASCII formatted sequence listing with a file named 097854-1317041-004010PC.txt, created on Jun. 10, 2022 and having a size of 649 bytes, and is filed concurrently with the specification. The sequence listing contained in this ASCII formatted document is part of the specification and is herein incorporated by reference in its entirety.

### FIELD

The present disclosure provides methods and compositions for the treatment of hypoglycemia, particularly congenital hyperinsulinism, and so relates to the fields of chemistry, medicinal chemistry, medicine, molecular biology, and pharmacology.

### BACKGROUND

Congenital hyperinsulinism (CHI) is a genetic disorder of pancreatic beta-cell function characterized by failure to suppress insulin secretion in the presence of hypoglycemia and particularly severe fasting-hypoglycemia. Some patients also present with protein-induced hyperinsulinemic hypoglycemia as well.

CHI first manifests in the neonatal period with persistent, life-threatening hypoglycemia characterized by seizures, lethargy, apnea and other symptoms resulting from neuroglycopenia and requiring hospitalization, in some instances in intensive care, and high glucose infusion requirements (e.g., through central lines). Severe hypoglycemia places infants at risk of death and causes irreparable brain damage and neurodevelopmental deficits in up to 50% of children due to inadequate treatment.

Initial management involves prolonged intensive care hospitalization and administration of high glucose infusion levels, putting patients at increased risk of heart failure due to fluid overload. Pancreatectomy is often undertaken to mitigate severe hypoglycemia and preserve brain function for these patients. However, pancreatectomy often exposes patients to other, high risk conditions. For example, long-term outcome studies have shown a high incidence of diabetes mellitus that is iatrogenic in nature and attributed to subtotal pancreatectomy. Apart from diabetes, pancreatectomy carries a high risk of other acute and long-term complications, such as bowel obstruction, malabsorption, and death.

In summary, CHI is a serious and life-threatening disease, as illustrated by the risk of death, high incidence of brain damage and neurodevelopmental deficits, and high incidence of diabetes and other devastating and permanent disease and treatment sequelae. Therefore, there is an urgent need for progress in development of safe, effective, and targeted pharmacotherapies for treatment of CHI.

### SUMMARY

The present disclosure provides methods of treating a subject having congenital hyperinsulinism (CHI), the method comprising administering a therapeutically effective amount of avexitide to the subject. In some instances, the avexitide is administered subcutaneously. In some instances, CHI is associated with a genetic abnormality, a mutation, or a syndrome. In some instances the avexitide is administered by subcutaneous injection. In some instances the avexitide is administered by intravenous infusion.

In one aspect, provided are methods of treating a subject with congenital hyperinsulinism (CHI), wherein the subject is an infant receiving continuous intravenous glucose infusion, the method comprising subcutaneously administering, optionally subcutaneously administering, a therapeutically effective amount of avexitide to the subject.

In another aspect, provided are methods of reducing a glucose infusion rate (GIR) for a subject having congenital hyperinsulinism (CHI), the method comprising administering a therapeutically effective amount of avexitide to the subject.

In another aspect, provided are methods of treating congenital hyperinsulinism (CHI) in a subject, the method comprising administering a total daily dose of 4 mg to 400 mg of avexitide to the subject.

In another aspect, provided are methods of reducing the hypoglycemia event rate for a subject having congenital hyperinsulinism (CHI), the method comprising administering therapeutically effective dose of avexitide to the subject, wherein the therapeutically effective dose is a total daily dose of 4 mg to 210 mg if the subject weighs 56 kg or less, optionally 2.3 to 56 kg, and is a total daily dose of 100 mg to 400 mg if the subject weighs more than 56 kg.

### BRIEF DESCRIPTION OF THE DRAWINGS

These figures are intended to be illustrative, not limiting. Although the aspects of the disclosure are generally described in the context of these figures, it should be understood that it is not intended to limit the scope of the disclosure to these particular aspects.

FIG. 1 illustrates the mechanism of action for avexitide in patients with loss-of-function mutations in the K_{ATP} channel.

FIG. 2 is a schematic showing the design of a study of avexitide for treatment of CHI in neonates and infants, as described in Example 1 and according to embodiments of this disclosure. In particular, the schematic identifies a randomized, single-ascending dose (100 to 1000 pmol/kg/min) study of avexitide and placebo by continuous IV infusion for up to 12 hours on two days administered to thirteen neonates and infants with diazoxide-unresponsive CHI in crossover design and random order. Plasma glucose was monitored every 30 minutes, with glucose infusion rate (GIR) adjusted hourly to maintain glucose in the range of 70-90 mg/dL. The primary endpoint was GIR, defined in the protocol as mean GIR during the final 2 hours of infusion and redefined for the current analyses as mean GIR during the final 6 hours of infusion.

FIG. 3 is a set of graphs showing the individual fitting of the concentration-time profiles of eleven neonate and infant patients treated with avexitide as described in Example 1 and according to embodiments of this disclosure. In the graphs, circles indicate observed avexitide plasma concentrations, black lines indicate population prediction of avexitide concentration-time profiles, and red lines indicate individual prediction of avexitide concentration-time profiles.

FIG. 4 is a set of graphs showing the general goodness-of-fit plots of the external validation for the treatment described in Example 1 and according to embodiments of this disclosure. In particular, FIG. 4 shows: (a) individual predicted (IPRED) plasma avexitide concentrations versus observed avexitide concentrations on a logarithmic scale (bottom left); (b) population predicted (PRED) plasma avexitide concentrations versus observed plasma avexitide concentrations on a logarithmic scale (top left); (c) conditional weighted residuals (CWRES) of plasma avexitide concentrations versus time (top right); and (d) CWRES versus PRED (bottom right). In these graphs, points are individual data, solid black lines represent the unit diagonal, and blue solid lines represent the unit line at zero.

FIG. 5 is a set of graphs showing the model-predicted individual avexitide concentration-time profiles following avexitide IV infusion for the treatment described in Example 1 and according to embodiments of this disclosure.

FIG. 6 is a graph showing the results of a model fitting of the pharmacokinetic/pharmacodynamics (PK/PD) relationship between avexitide concentration and absolute change in GIR for treatment described in Example 1 and according to embodiments of this disclosure. In FIG. 6, the black line indicates the model predicted mean response, the dark grey area indicates 90% CI, the light grey area indicates 90% PI, and the circles indicate observed absolute change in GIR (ng/mL).

FIGS. 7A-7B are graphs showing simulated avexitide plasma concentrations following subcutaneous injections of avexitide in neonate and infant patients with CHI according to embodiments of this disclosure. FIG. 7A shows simulated avexitide plasma concentrations following BID subcutaneous injections, and FIG. 7B shows simulated avexitide plasma concentrations following TID subcutaneous injections.

FIG. 8 is a schematic showing the design of a study of avexitide for treatment of CHI in neonates and infants, as described in Example 2 and according to certain embodiments of this disclosure. In particular, the schematic illustrates a double-blind, placebo-controlled, dose escalation, crossover study followed by an open-label period of avexitide in neonates and infants with CHI. A Run-In GIR will be established (mean GIR over an 8-hour period). Patients will be randomized to 2 treatment sequences (avexitide-placebo or placebo-avexitide) in a 1:1 ratio. All patients will receive SC injections of avexitide TID for 72 hours and SC injections of placebo TID for 72 hours in crossover design and random order.

### DETAILED DESCRIPTION

### I. Definitions

The terminology used herein is for the purpose of describing particular embodiments only, and is not intended to be limiting, because the scope of the present invention will be limited only by the appended claims. Unless defined otherwise, all technical and scientific terms used herein have the same meaning as commonly understood by one of ordinary skill in the art to which this invention belongs. In this specification and in the claims that follow, reference will be made to a number of terms that shall be defined to have the following meanings unless a contrary intention is apparent. In some cases, terms with commonly understood meanings are defined herein for clarity and/or for ready reference, and the inclusion of such definitions herein should not be construed as representing a substantial difference over the definition of the term as generally understood in the art.

Although any methods and materials similar or equivalent to those described herein can be used in the practice or testing of the present invention, the preferred methods and materials are now described. All technical and patent publications cited herein are incorporated herein by reference in their entirety. Nothing herein is to be construed as an admission that the invention is not entitled to antedate such disclosure by virtue of prior invention.

All numerical designations, e.g., pH, temperature, time, concentration, and molecular weight, including ranges, are approximations which are varied (+) or (−) by increments of 0.1 or 1.0, as appropriate. It is to be understood, although not always explicitly stated that all numerical designations are preceded by the term “about.” References to ranges include the end-points unless indicated otherwise. For example, administration of avexitide at a concentration of 30 mg/mL to 180 mg/mL includes administration of 30 mg/mL or 180 mg/mL.

The singular forms “a,” “an,” and “the” include plural referents unless the context clearly dictates otherwise. Thus, for example, reference to “a compound” includes a plurality of compounds.

The term “administration” refers to introducing a compound, a composition, or an agent of the present disclosure into a subject, such as a human. One preferred route of administration of the agents is subcutaneous administration. Other routes include including oral, intravenous, buccal, rectal, parenteral, intraperitoneal, intradermal, intratracheal, intramuscular, inhalational, and the like.

The term “baseline,” unless otherwise specified or apparent from context, refers to a measurement (of, e.g., weight, height, insulin secretion) made prior to a course of therapy.

The term “comprising” is intended to mean that the compounds, compositions and methods include the recited elements, but not excluding others. “Consisting essentially of” when used to define compounds, compositions and methods, shall mean excluding other elements that would materially affect the basic and novel characteristics of the claimed invention. “Consisting of” shall mean excluding any element, step, or ingredient not specified in the claim. Embodiments defined by each of these transition terms are within the scope of this invention.

The terms “course of treatment” and “course of therapy” are used interchangeably herein, and refer to the medical interventions made after a patient is diagnosed. Medical interventions include, without limitation, the administration of drugs for a period of time.

The term “formulation” or “pharmaceutical formulation,” as used herein, refers to a composition suitable for administration to a subject. A pharmaceutical formulation may be sterile, and preferably free of contaminants that are capable of eliciting an undesirable response within the subject (e.g., the compounds in the pharmaceutical formulation are pharmaceutical grade). Pharmaceutical formulations can be designed for administration to subjects or patients in need thereof via a number of different routes of administration, including oral, intravenous, buccal, rectal, parenteral, intraperitoneal, intradermal, intramuscular, subcutaneous, inhalational and the like. In some embodiments, a pharmaceutical formulation as described herein is formulated for subcutaneous administration.

The terms “patient” or “subject,” are used interchangeably and refer to an individual (e.g., a human or a non-human mammal). In some embodiments, a patient or subject has CHI. In some embodiments, a patient or subject has a condition that speeds transit to the ileum (early nutrient exposure to L-cells).

The term “pharmaceutical composition” is meant to encompass a composition suitable for administration to a subject. In general a “pharmaceutical composition” is sterile, and preferably free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, intravenous, buccal, rectal, parenteral, intraperitoneal, intradermal, intratracheal, intramuscular, subcutaneous, inhalational, and the like.

As used herein, a “therapeutically effective amount” is an amount of an active ingredient (e.g., avexitide or its pharmaceutically acceptable salt) that eliminates, ameliorates, alleviates, or provides relief of the symptoms or leads to clinical outcomes for which it is administered.

The terms “treatment”, “treating”, and “treat” are defined as acting upon a disease, disorder, or condition with an agent to reduce or ameliorate the pharmacologic and/or physiologic effects of the disease, disorder, or condition and/or its symptoms. In particular, the terms refer to administering avexitide to treat hypoglycemia (e.g., CHI). “Treatment,” as used herein, covers any treatment of a disease in a human subject, and includes: (a) reducing the risk of occurrence of the disease in a subject determined to be predisposed to the disease but not yet diagnosed as infected with the disease, (b) impeding the development of the disease, and/or (c) relieving the disease, i.e., causing regression of the disease and/or relieving one or more disease symptoms. “Treatment” is also meant to encompass delivery of an inhibiting agent to provide a pharmacologic effect, even in the absence of a disease or condition. For example, “treatment” encompasses delivery of an agent that provides for enhanced or desirable effects in the subject.

“QD,” “BID,” and “TID” have their usual meanings of administration of a composition (e.g., a formulation of avexitide) once per day, twice per day, or three times per day, respectively. In some embodiments, administration once per day (QD) means that at least 20 hours, at least 22 hours, or about 24 hours elapse between administrations. In some embodiments, administration once per day means administration about every 24 hours. In some embodiments, administration twice per day (BID) means that at least 4 hours, at least 6 hours, at least 8 hours, at least 10 hours, at least 11 hours, or about 12 hours elapse between administrations. In some embodiments, administration twice per day means administration about every 12 hours.

“Run-In Period” refers to a designated period after recruitment but before randomization into treatment groups during which all trial participants receive the same intervention of active treatment, placebo, or no intervention.

“Continuous glucose monitoring” (CGM) refers to a process of continuously tracking blood glucose levels in a subject. This is typically performed using a device worn by the subject that monitors blood glucose levels throughout the day and night.

“Self-monitoring of blood glucose” (SMBG) refers to a process in which a subject periodically measures their blood sugar levels. This is typically performed using a glucometer (glucose meter).

“Euglycemia” as used herein refers to a blood glucose concentration of 70 to 90 mg/dL (e.g., 70 to 80 mg/dL). In some instances, euglycemia refers to a blood glucose concentration of 70 to 80 mg/dL.

“Hypoglycemia” or “hypoglycemic” or the like are used herein to refer to a blood glucose level of less than 70 mg/dL, also measured as less than 3.9 mmol/L, in a subject. A “hypoglycemic event” is a period of time in which a subject is experiencing hypoglycemia. When a subject is experiencing hypoglycemia (i.e. a hypoglycemic event), they are referred to as “hypoglycemic.”

As used herein “glucose infusion rate” refers to an amount of glucose (dextrose) administered to a subject by infusion over time sufficient to maintain euglycemia in a subject with CHI.

“Clinically important hypoglycemia” as used herein to refers to a blood glucose level of less than 54 mg/dL, also measured as less than 3.0 mmol/L, in a subject.

“Severe hypoglycemia” as used herein refers to a hypoglycemia event in which a subject experiences altered mental functioning and/or altered physical functioning that requires assistance from another person for recovery, regardless of whether a patient actually receives external assistance.

“Time in Range” or “TIR” is defined as the percent time in the glucose range of 70 to 180 mg/dL (3.9 to 10.0 mmol/L), inclusive, as measured by CGM.

“Time Below Range Level 1” or “TBR Level 1” is defined as the percent time in hypoglycemia as measured by CGM, which is calculated as the total number of CGM data points that are <70 mg/dL, divided by the total number of CGM data points obtained from a patient within the same time period.

“Time Below Range Level 1 Nocturnal” or “TBR Level 1 Nocturnal” is defined as the percent time in nocturnal hypoglycemia during the period of time of 12:00 AM to 8:00 AM as measured by CGM. TBR Level 1 Nocturnal can be equated to the period of time during which a subject is typically asleep during a day, even if not during the hours of 12:00 AM to 8:00 AM.

“Hypoglycemia event rate (Level 1)” or “rate of hypoglycemia (Level 1)” and like phrasing are defined as the number of hypoglycemia events in a subject observed per week as measured by SMBG or as the number of hypoglycemia events observed per week as measured by CGM and sustained for at least 15 minutes. This definition is consistent with the use of the term as used by the European Association for the Study of Diabetes (EASD) and the American Diabetes Association (ADA).

“Clinically important hypoglycemia event rate (Level 2)” is defined as the number of clinically important hypoglycemia events in a subject observed per week as measured by CGM and sustained for at least 15 minutes, or as the number of clinically important hypoglycemia events in a subject observed per week as measured by SMBG. This definition is consistent with the use of the term as used by the European Association for the Study of Diabetes (EASD) and the American Diabetes Association (ADA).

“Time Below Range Level 2” or “TBR Level 2” is defined as the percent time a subject is experiencing clinically important hypoglycemia as measured by CGM.

“Severe hypoglycemia event rate (Level 3)” (EASD and ADA) is defined as the total number of severe hypoglycemia events observed per week.

### II. Methods of Treatment

The present disclosure provides methods of treating congenital hyperinsulinism (CHI). In some embodiments, the methods comprise administering avexitide or a formulation comprising avexitide to a subject having CHI. In some embodiments, provided are methods of treating a subject with CHI by subcutaneously administering a therapeutically effective dose of avexitide to the subject. In some instances, the provided methods are useful in treating CHI in an acute setting, such as in a hospital after birth, in order to stabilize blood glucose level of a subject having CHI. As described herein, in some embodiments, treatment with avexitide reduces the glucose infusion rate necessary to maintain euglycemia in a subject having CHI. The present disclosure also provides methods of reducing a glucose infusion rate (GIR) for a subject having congenital hyperinsulinism (CHI), the method comprising administering a therapeutically effective amount of avexitide to the subject. In some instances, the provided methods are useful for chronic, long-term treatment so as to maintain a stable blood glucose level of a subject having CHI. In some embodiments, administration can be subcutaneous or intravenous. In some instances, the methods comprise administering specific dosages of avexitide based on patient body weight. According to certain embodiments, a subject with CHI can be effectively treated by subcutaneous administration of avexitide. In particular, it has been determined that subcutaneous administration (e.g., BID or TID administration) of avexitide achieves therapeutic plasma levels of avexitide in subjects (e.g., children and/or neonates). In some embodiments, the therapeutic plasma levels of avexitide are in the range of 0.9 mg/kg-3.6 mg/kg. In some embodiments, administration of avexitide (e.g., subcutaneously) effectively reduces the GIR necessary to maintain euglycemia in subjects (e.g., children and/or neonates) having CHI.

Avexitide has demonstrated preliminary evidence of substantial improvement in safety profile over other pharmacotherapies used off-label. Off-label use of pharmacotherapy for treatment of CHI is often associated with poor efficacy and tolerability. Moreover, these medications can be associated with safety concerns. For example, diazoxide is ineffective for the majority of patients with CHI and is associated with hypertrichosis, fluid retention, pulmonary edema, pulmonary hypertension, neutropenia, thrombocytopenia, hyperuricemia, hyperglycemia, and GI effects, such as appetite suppression and vomiting. Another example is octreotide, which is not indicated for neonates and infants less than 2 months of age due to effects on splanchnic circulation and risk of necrotizing enterocolitis. In children older than 2 months old, octreotide can elevate liver enzymes, cause biliary sludging and gallstone formation, and occasionally lead to growth failure by reducing growth hormone secretion. Another example is IV glucagon, the use of which commonly causes peripheral line occlusion as a result of fibrillation of native glucagon, rendering continuous treatment not only ineffective but also unsafe. Catheter occlusion has also been observed during investigational administration by continuous SC infusion of glucagon. Glucagon has also been associated with erythema necrolyticum, which was observed in 4 out of 11 patients with CHI who were administered chronic doses of glucagon across 2 studies. Glucagon can also be associated with poor tolerability, including GI symptoms and vomiting. In addition, avexitide infusion has been shown to increase fasting blood glucose levels and reduce insulin/glucose AUC in patients. In a study of adolescents and adults with CHI, avexitide raised fasting blood glucose concentrations, reduced the requirement for glycemic rescue, and reduced fasting insulin/glucose ratios. See Calabria et al. (2012).

Provided herein are methods useful for acute treatment of hospitalized neonates and infants with CHI. In one aspect, provided are methods of treating a subject with congenital hyperinsulinism (CHI), wherein the subject is an infant having a central line for providing continuous intravenous glucose infusion at a GIR, the method comprising administering a therapeutically effective amount of avexitide to the subject. In another aspect, provided are methods of reducing a glucose infusion rate (GIR) for a subject having congenital hyperinsulinism (CHI), the method comprising administering a therapeutically effective amount of avexitide to the subject. As described herein, in some embodiments, treatment with avexitide reduces the glucose infusion rate necessary to maintain euglycemia in a subject having CHI. In some embodiments, the subject requires continuous glucose infusion. In some embodiments, the subject has a baseline GIR of 1 to 40 mg/kg/min. In some embodiments, the avexitide is administered subcutaneously. In some embodiments, the avexitide is administered intravenously. In some embodiments, the subject is less than 2 years old. In some instances, the subject is less than 1 year old. In some instances, the subject is a neonate (0-28 days old). In some instances, the subject is less than 14 days old. In some instances, the subject is 14 to 364 days old. Additional information on and embodiments relating to the patient population, avexitide, routes of administration and dosing regimens for these methods is provided below in Sections IIB, IIC, II.D and II.E.

Glucose infusion rate (GIR) is a measure of glucose requirements to maintain euglycemia in children with CHI. Maintenance of euglycemia in CHI patients often depends on high GIR (up to 20-25 mg/kg/min) by intravenous or central lines. This practice is frequently associated with severe complications including sepsis and fluid overload, which in the presence of the myocardial hypertrophy seen in affected children increases the risk for heart failure. Those patients whose GIR requirements exceed 10 mg/kg/min are not candidates for intragastric dextrose (via g-tube), a strategy frequently used to manage the hypoglycemia and prevent the need for a pancreatectomy, because rates exceeding this threshold involve excessively high osmotic loads to the gut, causing poor absorption and tolerance (Vajravelu 2019). Thus, children with GIR requirements of greater than 10 mg/kg/min often undergo subtotal (98%) pancreatectomy to lower GIR rates, leading to prolonged hospitalization, life-threatening complications, and ultimately induction of insulin-dependent diabetes. Among these subject groups, pancreatectomy is a common approach to mitigate severe hypoglycemia. In some cases, the methods described herein may eliminate the need for pancreatectomy or reduce the degree of pancreatectomy required.

In some embodiments, the treatment reduces the mean GIR of the subject during the treatment period (i.e. a 24 hour period of time that the subject is being treated with avexitide). In some embodiments, the treatment reduces the mean GIR of the subject by the end of the treatment period (i.e. a 24 hour period of time that the subject is being treated with avexitide). In some instances, reduction in mean GIR is achieved after both doses of a BID dosing regimen are administered. In some instances, reduction in mean GIR is achieved after all three doses of a TID dosing regimen are administered. In some instances, reduction in mean GIR is achieved once avexitide reaches steady state plasma concentration. In some instances, reduction in mean GIR is achieved after a bolus dose and at least one additional dose (i.e. maintenance dose) are administered. In some embodiments, the treatment reduces the mean GIR of the subject during a final segment of the treatment period. In some embodiments, the treatment reduces the mean GIR of the subject during the final 2 hours, the final 3 hours, the final 4 hours, the final 5 hours, the final 6 hours, the final 7 hours, the final 8 hours, the final 10 hours, the final 12 hours, the final 14 hours, the final 16 hours, the final 18 hours, the final 20 hours, or the final 22 hours of the treatment period.

In some embodiments, the treatment method reduces the GIR of the subject by 5% to 100% as compared to treatment with a placebo or as compared to baseline GIR, e.g., 5% to 90%, 5% to 80%, 5% to 70%, 5% to 60%, 5% to 50%, 10% to 100%, 10% to 90%, 10% to 80%, 10% to 70%, 10% to 60%, 10% to 50%, 15% to 100%, 15% to 90%, 15% to 80%, 15% to 70%, 15% to 60%, 15% to 50%, 20% to 100%, 20% to 90%, 20% to 80%, 20% to 70%, 20% to 60%, 20% to 50%, 25% to 100%, 25% to 90%, 25% to 80%, 25% to 70%, 25% to 60%, 25% to 50%, 30% to 90%, 30% to 60%, 40% to 60%, 40% to 90%, 50% to 90%, 60% to 90%, 40% to 100%, 45% to 100%, 50% to 100%, 55% to 100%, 60% to 100%, 65% to 100%, 70% to 100%, 75% to 100%, 80% to 100%, 85% to 100%, 90% to 100%, or 95% to 100%. For example, the GIR may be reduced by any of 5%, 10%, 15%, 20%, 25%, 30%, 35%, 40%, 45%, 50%, 55%, 60%, 65%, 70%, 75%, 80%, 85%, 90%, 95%, or 100%. In some instances, the methods reduce the GIR of the subject by 15% to 60%. In some instances, the methods reduces the GIR to 0. In some instances, the subject has a baseline GIR of 1 to 40 mg/kg/min. In terms of lower limits, the treatment method may reduce the GIR by at least 5% as compared to treatment with a placebo or as compared to baseline GIR, e.g., at least 10%, at least 15%, at least 20%, or at least 25%. In terms of upper limits, the treatment method may reduce the GIR as compared to treatment with a placebo or as compared to baseline GIR by up to 100%, e.g., up to 90%, up to 80%, up to 70%, up to 60%, or up to 50%. In some embodiments, the reduction is the GIR is compared to treatment with a placebo. In some embodiments, the reduction is the GIR is compared to baseline GIR.

In some embodiments, the treatment method reduces the GIR of the subject to less than or equal to 10 mg/kg/min, e.g., less than 9 mg/kg/min, less than 8 mg/kg/min, less than 7 mg/kg/min, less than 6 mg/kg/min, or less than 5 mg/kg/min. In some embodiments, the treatment method may reduce the GIR to 0. Said another way, in some embodiments, the treatment method eliminates the need to administer glucose to maintain euglycemia.

Administering avexitide, as described herein, and thereby reducing the GIR of the subject presents a number of benefits and advantages. Reduction in GIR beneficially reduces the volume of dextrose infused, thereby lowering the risk of complications from fluid overload during inpatient care and prior to partial pancreatectomy (in the case of focal disease) or subtotal pancreatectomy (in the case of diffuse disease). This is particularly true among children and/or neonates. In some cases, reduction in GIR can allow for a less extensive pancreatectomy (e.g., removal of 90% vs. 98% of the pancreas) and therein delay onset of insulin-dependent diabetes, for example, by 5-10 years. Furthermore, a reduction in GIR to less than or equal to 10 mg/kg/min can allow for the removal of intravenous or central lines and/or a transition to enteral dextrose support. In some cases, a reduction of GIR to less than or equal to 10 mg/kg/min can allow for (earlier) hospital discharge and/or avoidance of pancreatectomy.

Prior to the studies described herein, the effectiveness of avexitide in reducing the GIR needed to maintain euglycemia has not been previously established. Nor had the effectiveness of avexitide in achieving the benefits associated with reduced GIR, including lowering the risk of complications from fluid overload during inpatient care and prior to partial pancreatectomy (in the case of focal disease) or subtotal pancreatectomy (in the case of diffuse disease), reduced extent of pancreatectomy, removal of intravenous or central lines and/or a transition to enteral dextrose support, and/or the avoidance of pancreatectomy.

In some embodiments, administering avexitide to the subject permits stopping continuous glucose infusion for the subject. In some embodiments, administering avexitide to the subject permits stopping continuous glucose infusion for the subject (e.g., removal of central or peripheral line removal). In some embodiments, administering avexitide to the subject reduces at least one of the total amount or volume of carbohydrate required to be administered to the subject to maintain euglycemia or the need for IV carbohydrate rescue of the subject. In some embodiments, the carbohydrate is dextrose/glucose. In some embodiments, administering avexitide to the subject at least one of hypoglycemia event rate or clinically important hypoglycemia event rate. In some embodiments, administering avexitide to the subject reduces time to stopping continuous glucose infusion (e.g., central or peripheral line removal), and optionally, time to discharge readiness. In some embodiments, administering avexitide to the subject permits avoidance of performing a pancreatectomy on the subject.

Provided herein are methods useful for long term treatment of CHI patients to maintain euglycemia. In one aspect, provided are methods of treating congenital hyperinsulinism (CHI) in a subject, the method comprising subcutaneously administering a total daily dose of 4 mg to 400 mg of avexitide to the subject. In another aspect, provided are methods of reducing the hypoglycemia event rate for a subject having CHI, the method comprising administering, optionally by subcutaneously administering, a total daily dose of 4 mg to 400 mg of avexitide to the subject. In some embodiments, the subject has documented uncontrolled hypoglycemia but does not require continuous glucose infusion. In some embodiments, the therapeutically effective dose is a total daily dose of 4 mg to 210 mg if the subject weighs 56 kg or less, optionally 2.3 to 56 kg. In some embodiments, the therapeutically effective dose is a total daily dose of 50 mg to 400 mg if the subject weighs more than 56 kg. In some embodiments, the avexitide is administered to such patients twice daily (BID). In some embodiments, the avexitide is administered to such patients subcutaneously. In some embodiments, the avexitide is administered to such patients by intravenous infusion. Additional information on and embodiments relating to the patient population, avexitide, routes of administration and dosing regimens for these methods is provided below in Sections II.B, II.C, II.D and II.E.

In some embodiments, avexitide treatment in such patients results in maintenance of euglycemia in the subject. In some embodiments, avexitide treatment reduces in such subjects at least one of hypoglycemia event rate, clinically important hypoglycemia event rate, or severe hypoglycemia event rate. In some embodiments, avexitide treatment reduces in such subjects at least one of TBR Level 1, TBR Level 1 Nocturnal, TBR Level 2, or TIR. In some embodiments, avexitide treatment permits reduction in such subjects of at least one of total carbohydrates administered via oral route, nasogastric tube, or gastrostomy tube per week to treat or prevent hypoglycemia events. In some embodiments, avexitide treatment permits reduction in such subjects of total nightly carbohydrates administered. In some embodiments, avexitide treatment is such patients permits removal of a nasogastric tube or gastrostomy tube from the subject.

In some embodiments, administration of avexitide reduces risk of heart failure in the subject. In some embodiments, administration of avexitide reduces risk of sepsis in the subject. In some embodiments, administration of avexitide reduces the requirement for pancreatectomy for the subject. In some embodiments, administration of avexitide reduces time to hospital discharge for the subject. In some embodiments, administration of avexitide reduces time to central or peripheral line removal in the subject. In some embodiments, administration of avexitide reduces risk for brain damage or related neurodevelopmental outcomes in the subject. In some embodiments, administration of avexitide reduces risk of death for the subject. In some embodiments, administration of avexitide reduces risk for fluid overload for the subject. In some embodiments, administration of avexitide reduces risk for seizure in the subject. In some embodiments, administration of avexitide permits reducing the dose of another drug, e.g., somatostatin analogue (such as octreotide or lanreotide) or diazoxide in the subject. In some embodiments, administration of avexitide reduces the extent of pancreatectomy in the subject (e.g., 90% pancreatectomy rather than 98% pancreatectomy). In some embodiments, administration of avexitide increases time to development of insulin-dependent diabetes in the subject. In some embodiments, administration of avexitide reduces the requirement for diuretic use (to protect against heart failure) in the subject.

### A. Congenital Hyperinsulinism

CHI arises from inherited or de novo mutations of genes involved in the regulation of insulin secretion. Mutations in more than 11 genes have been identified as associated with monogenic forms of CHI (see Table 1). The mechanism of action for avexitide in treating CHI in islets isolated from SUR1−/− mice as well as in pancreatic specimens from infants with K^{ATP} CHI has been determined. See De Leon et al. (2008) and Calabria et al. (2012). Syndromic genetic forms of CHI have also been identified, such as Beckwith-Wiedemann, Kabuki, and Turner syndromes.

TABLE 1

| Gene | Protein | Description |
|---|---|---|
| ABCC8 (ATP-binding | SUR1 | Sulfonylurea receptor; subunit of the K_{ATP} channel; regulation |
| cassette subfamily C, | | of channel gating |
| member 8) | | |
| KCNJ11 (potassium inward | Kir6.2 | Subunit of the K_{ATP} channel; regulation of channel gating |
| rectifying channel, | | |
| subfamily J, member 11) | | |
| SLC16A1 | MCT1 | Monocarboxylate transporter 1; mediates the transport of |
| | | lactate and pyruvate across cell membranes |
| GLUD-1 (glutamate | GDH | Glutamate dehydrogenase |
| dehydrogenase 1) | | |
| GCK | GCK | Glucokinase; has a central role in nitrogen metabolism |
| HADH (hydroxyacyl- | SCHAD | Short chain 3-hydroxyacyl-CoA dehydrogenase functioning |
| coenzyme A | | as inhibitor of glutamate dehydrogenase through direct |
| dehydrogenase) | | protein/protein interaction |
| UCP2 | UCP2 | Uncoupling protein 2; control of pathway involved in |
| | | dissipation of the proton electrochemical gradient across the |
| | | inner mitochondrial membrane |
| HK1 | HK1 | Hexokinase 1; catalyzes the first step in glucose metabolism |
| | | us ATP for the phosphorylation of glucose to glucose-6- |
| | | phosphate |
| PGM1 | PGM1 | Phosphoglucomutase; catalyzes the transfer of phosphate |
| | | between the 1 and 6 positions of glucose |
| HNF1A | HNF1- | Hepatic nuclear transcription factor 1-alpha; transcriptional |
| | alpha | activator that regulates genes largely involved in the hepatic |
| | | gluconeogenic program and lipid metabolism |
| HNF4A | HNF₄- | Hepatic nuclear transcription factor 4-alpha; binds to DNA |
| | alpha | sites required for the transcription of alpha 1-antitrypsin, |
| | | apolipoprotein CIII, transthyretin genes, and HNF1-alpha |

Sources: Stanley 2016; Galcheva et al 2019, Ackermann and Palladino 2015

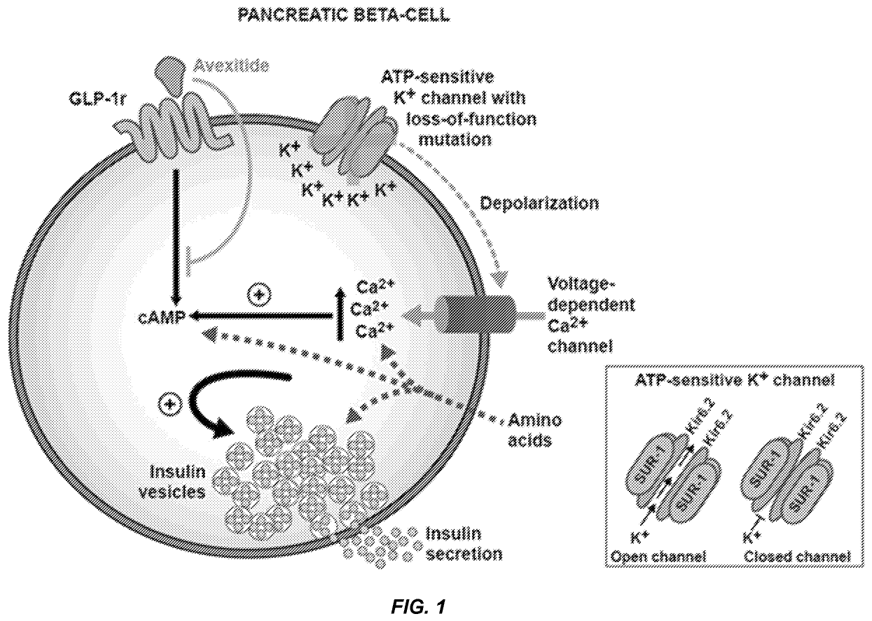

FIG. 1 describes the mechanism of action for avexitide in patients with loss-of-function mutations in the K_{ATP} channel. Briefly, in healthy individuals, beta-cell K_{ATP} channels couple the metabolic state of the beta-cell to the cell's membrane potential by responding to changes in intracellular ATP concentrations. In the presence of glucose, K_{ATP} channels close due to a rise in the ATP/adenosine diphosphate ratio, leading to beta-cell depolarization, activation of voltage-gated calcium channels, an increase in cytosolic concentrations of calcium, and exocytosis of insulin-containing granules. However, in affected patients, a loss-of-function mutation of the K_{ATP} channel leads to persistent beta-cell membrane depolarization, cytosolic calcium accumulation, and insulin degranulation, causing fasting- and protein-induced hypoglycemia regardless of plasma glucose concentrations.

Preclinical studies conducted in a mouse model of CHI due to mutations in K_{ATP} channel genes (K_{ATP} CHI) and in patients with CHI suggest a role for GLP-1r signaling in catalyzing calcium-mediated insulin degranulation in K_{ATP} CHI. GLP-1r signaling activates adenylyl cyclase and generation of cyclic adenosine monophosphate (cAMP). Inhibition of GLP-1r signaling has been shown to reduce cAMP accumulation and insulin degranulation, thereby demonstrating a role for GLP-1r signaling in the pathophysiology of K_{ATP} CHI (FIG. 1). K_{ATP} channels, composed of Kir6.2 and SUR-1, couple the metabolic state of the beta-cell to the membrane potential by sensing changes in intracellular ATP concentrations. Loss-of-function K_{ATP} channel mutations lead to membrane depolarization and activation of voltage-gated calcium channels with a rise in cytosolic calcium. Basal and ligand-stimulated GLP-1r signaling raises cAMP levels, promoting calcium-mediated insulin degranulation.

The most common and severe form of CHI is known as K_{ATP} CHI results from inactivating (loss-of-function) mutations in one of two adjacent genes located on chromosome 11p15.1; ABCC8 and KCNJ11. These genes encode the sulfonylurea receptor 1 (SUR-1) and a K⁺-selective pore-forming subunit (Kir6.2), which together form the ATP-sensitive potassium channel (K_{ATP} channel) on the plasma membrane of pancreatic beta-cells. In healthy individuals, K^{ATP} channels couple the metabolic state of the beta-cell to the membrane potential by responding to changes in intracellular ATP concentrations. In the presence of glucose, K^{ATP} channels close due to a rise in the ATP/ADP ratio, leading to beta-cell depolarization, activation of voltage-gated calcium channels, an increase in cytosolic concentrations of calcium, and exocytosis of insulin-containing granules. The loss of K_{ATP} channel activity leads to persistent beta-cell membrane depolarization and insulin release, regardless of plasma glucose levels. in affected patients with CHI, loss-of-function mutation of the K^{ATP} channel leads to persistent beta-cell membrane depolarization, cytosolic calcium accumulation and insulin degranulation, causing fasting and amino acid-induced hypoglycemia regardless of plasma glucose concentrations. In the absence of functional K_{ATP} channels, hypoglycemia is usually unresponsive to medical treatment with diazoxide, a K_{ATP} channel agonist that is otherwise the mainstay of therapy for hyperinsulinism. K_{ATP} channel defect mutations account for approximately 97% of children with diazoxide unresponsive CHI.

K_{ATP} mutations are inherited in a recessive or a dominant manner. In children with recessive K_{ATP} mutations, subunit K_{ATP} channel proteins do not traffic to the beta-cell surface, resulting in either diffuse disease (diffuse CHI) involving the entire pancreas or focal disease (focal CHI) involving discrete and potentially resectable pancreatic lesions of islet cell adenomatosis, depending upon the pattern of inheritance. Diffuse CHI is caused by biallelic recessive mutations. Focal CHI is caused by paternal transmission of a monoallelic recessive mutation followed by a somatic loss of maternal chromosome 11p15.1. In children with dominant K_{ATP} mutations, subunit proteins traffic normally to the beta-cell surface but form K_{ATP} channel complexes at the cell surface that are impaired with varying degrees of functionality. Consequently, children with dominant K_{ATP} mutations may either be responsive or unresponsive to diazoxide, depending upon the degree of residual K_{ATP} channel activity.

### B. Patient Population

In some embodiments, a subject to be treated according to the methods described herein is a subject having congenital hyperinsulinism (CHI). Congenital hyperinsulinism is a disorder defined by excess production and/or secretion of insulin, which in turn causes hypoglycemia. In particular, CHI is characterized by the failure to suppress insulin secretion in the presence of hypoglycemia. In some embodiments, the subject to be treated exhibits excess production and/or secretion of insulin, e.g., in the presence of hypoglycemia. Several forms of congenital hyperinsulinism have been recognized. These include diazoxide-responsive and diffuse K_{ATP} hyperinsulinism, focal K_{ATP} hyperinsulinism, GHD-hyperinsulinism, glucokinase hyperinsulinism, and HNF1A and 4a defects.

In some embodiments, the subject who is treated is a human subject. In some embodiments, the subject is a pediatric subject. In some embodiments, the subject is a child. In some embodiments, the subject is a juvenile. In some embodiments, the subject is a neonate (newborn) (e.g., aged 0-28 days). In some instances, the subject is less than 14 days old. In some instances, the subject is less than 28 days old. In some instances, the subject is 14 to 364 days old. In some embodiments, the subject is an infant (e.g., aged 2 years or younger). In some instances, the subject is less than 2 years old. In some instances, the subject is less than 1 year old. In some embodiments, the subject is 1 year old to 12 years old. In some embodiments, the subject is 2 years old to 12 years old. In some embodiments, the subject is an adolescent (e.g., aged 18 years or younger). In some embodiments, the subject is an adult (e.g., 18 years or older). In some embodiments, the subject is any other type of subject known in the art. Each possibility represents a separate embodiment of the present invention.

In some embodiments, the subject has diffuse or focal disease. In some embodiments, the subject has diffuse disease. In some embodiments, the subject has focal disease. In some embodiments, a subject with focal disease can be cured by surgical therapy to remove the focal lesion (lesionectomy). In some instances, the subject has focal disease but has not yet received surgical therapy. In some instances, the subject has focal disease and has received focal lesionectomy but is still experiencing hypoglycemia. In some instances, the subject has focal disease but surgical therapy is not advised (e.g., due to focal lesion location).

In some embodiments, the subject has not had a pancreatectomy. In some embodiments, the subject has had at least a partial pancreatectomy. In some embodiments, the subject had had 50%, 60%, 70%, 80%, 85%, 90%, 95%, 98%, 99%, or 100% of their pancreas removed.

In some embodiments, the subject has a nasogastric tube or a gastrostomy tube. In some embodiments, the subject does not have a nasogastric tube or a gastrostomy tube.

Diazoxide can increase blood glucose by inhibiting insulin release in the pancreas. In some embodiments, the subject is unresponsive to medical treatment with diazoxide. In some embodiments, the subject is responsive to medical treatment with diazoxide. In some embodiments, the subject is responsive to medical treatment with octreotide. In some embodiments, the subject is unresponsive to medical treatment with octreotide. Octeotide is a synthetic version of the hormone somatostatin that can inhibit insulin and glucagon secretion in the pancreas.

In some instances, a diagnosis of CHI is confirmed for a subject to be treated based on genetic, clinical, and diagnostic workup consistent with the standard of care for patients with CHI.

In some embodiments, the CHI is associated with a genetic abnormality. In some embodiments, the CHI is associated with a genetic mutation. In some embodiments, the CHI is a result of a genetic abnormality. In some embodiments, the CHI is a result of a genetic abnormality, mutation, and/or syndrome. Germline mutations in several genes have been associated with CHI. genes encoding the sulfonylurea receptor (SUR-1), genes encoding an inward rectifying potassium channel (Kir6.2), genes encoding glucokinase (GCK), genes encoding glutamate dehydrogenase (GLUD-1), and genes encoding short-chain L-3-hydroxyacyl-CoA (SCHAD). In some embodiments, the genetic abnormality or mutation is a mutation in a gene encoding a sulfonylurea receptor (SUR-1), a gene encoding glucokinase (GCK), a gene encoding glutamate dehydrogenase (GLUD-1), a gene encoding mitochondrial enzyme short-chain 3-hydroxyacyl-CoA dehydrogenase (HADHSC), and/or any of the genes listed in Table 1. In some embodiments, the CHI is associated with any other mutation known in the art to be associated with a CHI. Each possibility represents another embodiment of the present invention.

In some embodiments, the subject's CHI is associated with a KATP channel dysfunction. In some embodiments, the CHI is a KATP hyperinsulinism. As noted above, the KATP channel is composed of Kir6.2 and SUR-1. Dysfunction of the KATP channel may be associated with dysfunction in one or both of Kir6.2 and SUR-1. In some embodiments, the CHI is associated with a mutation in a gene encoding a sulfonylurea receptor (ABCC8). In some embodiments, the CHI is associated with a mutation in a gene encoding an inward rectifying potassium channel, Kir6.2 protein (KCNJII). In some embodiments, the CHI is associated with a mutation in a gene or genes encoding a sulfonylurea receptor and/or a Kir6.2 protein.

In some embodiments, the subject's CHI is associated with a mutation in a gene encoding a glucokinase (GCK). In some embodiments, the CHI is associated with a mutation in a gene encoding a glutamate dehydrogenase (GLUD-1). In some embodiments, the CHI is associated with a mutation in a gene encoding a mitochondrial enzyme short-chain 3-hydroxyacyl-CoA dehydrogenase (HADHSC).

In some instances, a diagnosis of CHI is confirmed for a subject to be treated based on clinical and diagnostic workup consistent with the standard of care for patients with CHI, inclusive of at least one of the following criteria during hypoglycemia: hyperinsulinemia, hypofattyacidemia, hypoketonemia, or a glycemic response. In some instances, hyperinsulinemia occurs when plasma insulin is above the limit of detection of the assay documented during an event of hypoglycemia. In some instances, hypofattyacidemia occurs when the plasma free fatty acid is less than 1.7 mmol/L. In some instances, hypoketonemia occurs when plasma beta-hydroxybutyrate is less than 1.8 mmol/L. In some instances, a glycemic response occurs when there is an increase in plasma glucose of greater than 30 mg/dL (greater than 1.7 mmol/L) after IV or intramuscular glucagon administration. In some embodiments, the subject during hypoglycemia has at least one of hyperinsulinemia, hypofattyacidemia, hypoketonemia, or a glycemic response.

In some embodiments, the subject requires continuous glucose infusion, e.g., has a central or peripheral line for providing continuous intravenous glucose infusion. In some embodiments, the subject does not require continuous intravenous glucose infusion. In some embodiments, the subject has documented uncontrolled hypoglycemia but does not require continuous glucose infusion. In some instances the subject has documented uncontrolled hypoglycemia but can be managed in the outpatient setting. In some instances, uncontrolled hypoglycemia can be a subject experiencing an average of at least three documented episodes of hypoglycemia (e.g., hypoglycemia (Level 1)) per week as measured by SMBG checks.

As discussed above, severe hypoglycemia present particular risk for children and neonates, who face risks of death, brain damage, and neurodevelopmental deficits. The methods described herein may therefore be particularly beneficial for children (e.g., subjects aged 12 years or younger) and/or neonates (e.g., subject aged 1 year or younger).

In some embodiments, the subject is aged 0 days (e.g., newborn) to 1 month, e.g., 0 days to 14 days, 14 days to 4 weeks, 1 day to 4 weeks, 2 days to 4 weeks, 3 days to 4 weeks, 4 days to 4 weeks, 5 days to 4 weeks, 6 days to 4 weeks, 1 week to 2 weeks, 1 week to 3 weeks, 1 week to 4 weeks, 2 weeks to 3 weeks, 2 weeks to 4 weeks, or 3 weeks to 4 weeks.

In some embodiments, the subject is an age older than 1 month up to 18 years of age, e.g., 1 month to 12 years, 5 weeks to 18 years, 5 weeks to 12 years, 5 weeks to 10 years, 5 weeks to 8 years, 5 weeks to 6 years, 5 weeks to 4 years, 5 weeks to 2 years, 5 weeks to 1 year, 2 months to 18 years, 2 months to 12 years, 2 months to 10 years, 2 months to 8 years, 2 months to 6 years, 2 months to 4 years, 2 months to 2 years, 2 months to 1 year, 3 months to 18 years, 3 months to 12 years, 3 months to 10 years, 3 months to 8 years, 3 months to 6 years, 3 months to 4 years, 3 months to 2 years, 3 months to 1 year, 4 months to 18 years, 4 months to 12 years, 4 months to 10 years, 4 months to 8 years, 4 months to 6 years, 4 months to 4 years, 4 months to 2 years, 4 months to 1 year, 6 months to 18 years, 6 months to 12 years, 6 months to 10 years, 6 months to 8 years, 6 months to 6 years, 6 months to 4 years, 6 months to 2 years, 6 months to 1 year, 8 months to 18 years, 8 months to 12 years, 8 months to 10 years, 8 months to 8 years, 8 months to 6 years, 8 months to 4 years, 8 months to 2 years, 8 months to 1 year.

In some embodiments, the subject is 1 year to 18 years of age, e.g., 1 year to 17 years, 1 year to 16 years, 1 year to 15 years, 1 year to 14 years, 1 year to 13 years, 1 year to 12 years, 1 year to 11 years, 1 year to 10 years, 1 year to 9 years, 1 year to 8 years, 1 year to 7 years, 1 year to 6 years, 1 year to 5 years, 1 year to 4 years, 1 year to 3 years, 1 year to 2 years, 2 years to 12 years, 2 years to 11 years, 2 years to 10 years, 2 years to 9 years, 2 years to 8 years, 2 years to 5 years, 3 years to 12 years, 3 years to 11 years, 3 years to 10 years, 3 years to 9 years, 3 years to 8 years, 4 years to 12 years, 4 years to 11 years, 4 years to 10 years, 4 years to 9 years, 4 years to 8 years, 5 years to 12 years, 5 years to 11 years, 5 years to 10 years, 5 years to 9 years, or 5 years to 8 years, 6 years to 12 years, 10 years to 12 years, or 12 years to 18 years.

In terms of upper limits, the subject can be under 18 years of age, e.g., under 17 years, under 16 years, under 15 years, under 14 years, under 13 years, under 12 years, under 11 years, under 10 years, under 9 years, under 8 years, under 7 years, under 6 years, under 5 years, under 4 years, under 3 years, under 2 years, under 18 months, under 1 year, under 10 months, under 8 months, under 6 months, under 4 months, under 3 months, under 2 months, under 1 month, under 8 weeks, under 7 weeks, under 6 weeks, under 5 weeks, or under 4 weeks. In terms of lower limits, the subject a neonate, i.e. first day of life (day 0) or older, e.g., over 1 day, over 2 days, over 3 days, over 4 days, over 5 days, over 1 week, over 2 weeks, over 3 weeks over 4 weeks, over 1 month, over 2 months, over 3 months, over 4 months, over 6 months of age, over 1 year, over 2 years, over 3 years, over 5 years, over 7 years, over 10 years, over 12 years, over 18 years.

In some embodiments, the subject is a child over 12 years old or an adult, e.g., having an age of at least 12 years, 15 years, 18 years, 20 years, 30 years, 40 years, 50 years, 60 years, 65 years, or 70 years.

In some embodiments, the subject requires administration (e.g., infusion) of glucose to maintain euglycemia (e.g., a blood glucose concentration of 70 to 90 mg/dL or, more narrowly, 70 to 80 mg/dL). In some instances, the subject, such as an infant subject, is receiving continuous intravenous glucose infusion (e.g., via a central or peripheral line) prior to administration of avexitide. In some embodiments, the subject can have a baseline glucose infusion rate (GIR) to maintain euglycemia. Thus, the baseline GIR for a subject refers to the GIR of a subject before initiation of treatment with avexitide according to embodiments of the present disclosure.

In some embodiments, the subject has a baseline GIR of 1 to 50 mg/kg/min, e.g., 1 to 38 mg/kg/min, 1 to 36 mg/kg/min, 1 to 34 mg/kg/min, 1 to 32 mg/kg/min, 1 to 30 mg/kg/min, 2 to 40 mg/kg/min, 2 to 38 mg/kg/min, 2 to 36 mg/kg/min, 2 to 34 mg/kg/min, 2 to 32 mg/kg/min, 2 to 30 mg/kg/min, 2 to 25 mg/kg/min, 2 to 20 mg/kg/min, 2 to 15 mg/kg/min, 2 to 10 mg/kg/min, 4 to 40 mg/kg/min, 4 to 38 mg/kg/min, 4 to 36 mg/kg/min, 4 to 34 mg/kg/min, 4 to 32 mg/kg/min, 4 to 30 mg/kg/min, 4 to 25 mg/kg/min, 4 to 20 mg/kg/min, 4 to 15 mg/kg/min, 4 to 10 mg/kg/min, 5 to 40 mg/kg/min, 5 to 38 mg/kg/min, 5 to 36 mg/kg/min, 5 to 34 mg/kg/min, 5 to 32 mg/kg/min, 5 to 30 mg/kg/min, 5 to 25 mg/kg/min, 5 to 20 mg/kg/min, 5 to 15 mg/kg/min, 5 to 10 mg/kg/min, 6 to 40 mg/kg/min, 6 to 38 mg/kg/min, 6 to 36 mg/kg/min, 6 to 34 mg/kg/min, 6 to 32 mg/kg/min, 6 to 30 mg/kg/min, 6 to 25 mg/kg/min, 6 to 20 mg/kg/min, 6 to 15 mg/kg/min, 6 to 10 mg/kg/min, 8 to 40 mg/kg/min, 8 to 38 mg/kg/min, 8 to 36 mg/kg/min, 8 to 34 mg/kg/min, 8 to 32 mg/kg/min, 8 to 30 mg/kg/min, 8 to 25 mg/kg/min, 8 to 20 mg/kg/min, 8 to 15 mg/kg/min, 10 to 40 mg/kg/min, 10 to 38 mg/kg/min, 10 to 36 mg/kg/min, 10 to 34 mg/kg/min, 10 to 32 mg/kg/min, 10 to 30 mg/kg/min, 10 to 25 mg/kg/min, 10 to 20 mg/kg/min, 15 to 40 mg/kg/min, 15 to 38 mg/kg/min, 15 to 36 mg/kg/min, 15 to 34 mg/kg/min, 15 to 32 mg/kg/min, 15 to 30 mg/kg/min, 15 to 25 mg/kg/min, 15 to 20 mg/kg/min, 20 to 40 mg/kg/min, 20 to 38 mg/kg/min, 20 to 36 mg/kg/min, 20 to 34 mg/kg/min, 20 to 32 mg/kg/min, 20 to 30 mg/kg/min, 20 to 25 mg/kg/min, 25 to 40 mg/kg/min, 25 to 38 mg/kg/min, 25 to 36 mg/kg/min, 25 to 34 mg/kg/min, 25 to 32 mg/kg/min, 30 to 40 mg/kg/min, 35 to 40 mg/kg/min, or 25 to 50 mg/kg/min. In some embodiments, the subject has a baseline GIR or 5 to 15 mg/kg/min. In terms of lower limits, the subject may have a baseline GIR greater than 1 mg/kg/min, e.g., greater than 2 mg/kg/min, greater than 4 mg/kg/min, greater than 6 mg/kg/min, or greater than 8 mg/kg/min. In terms of upper limits, the subject may have a baselines GIR less than 40 mg/kg/min, less than 38 mg/kg/min, less than 36 mg/kg/min, less than 34 mg/kg/min, less than 32 mg/kg/min, or less than 30 mg/kg/min.

### C. Avexitide

Avexitide represents a targeted therapeutic approach for treatment of CHI based on the underlying disease pathophysiology and the mechanism of action of avexitide. Avexitide is a first-in-class glucagon-like peptide-1 receptor (GLP-1r) antagonist with inverse agonist properties. Avexitide binds to the GLP-1r and competes with endogenous GLP-1. As an inverse agonist, avexitide also prevents basal GLP-1r signaling. In the absence of avexitide, basal GLP-1r signaling activates adenyl cyclase and generation of cAMP. Inhibition of basal GLP-1r signaling by avexitide inverse agonism has been shown to reduce cAMP accumulation and insulin degranulation.

Avexitide, which is also referred to as exendin (9-39), is a 31 amino acid peptide with an empirical formula of C149H234N40047S and a molecular weight of 3369.8 Daltons. Avexitide comprises residues 9-39 of the GLP-1 receptor agonist exendin-4 and is a GLP-1 receptor antagonist with inverse agonist properties. See Montrose-Rafizadeh et al., Journal of Biological Chemistry, 272:21201-21206 (1997). The amino acid sequence for avexitide is shown as follows: H-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH₂ (SEQ ID NO: 1). Avexitide has a predicted isoelectric point of 4.69 and has a net charge of −1 at pH 6 that increases to a net charge of +4 at pH 3.0. As used herein, the term “avexitide” also encompasses pharmaceutically acceptable salts of avexitide (exendin (9-39)), including but not limited to sulfate, hydrochloride, phosphate, sulfamate, acetate, citrate, lactate, tartrate, methanesulfonate, ethanesulfonate, benzenesulfonate, p-toluenesulfonate, cyclohexylsulfamate and quinate salts. In some embodiments, avexitide is in the form of an acetate or trifluoroacetate salt. In some embodiment, the avexitide is avexitide acetate. Avexitide and pharmaceutically acceptable salts thereof are commercially available (e.g., Bachem). Variants of avexitide retaining GLP-1 receptor antagonist activity are also useful in the disclosed methods.

In some embodiments, the avexitide administered to the subject comprises an amino acid sequence having at least 90% identity to SEQ ID NO:1. In some embodiments, the avexitide comprises the amino acid sequence of SEQ ID NO:1. In some embodiments, the avexitide consists of the amino acid sequence of SEQ ID NO:1. The term “identity” or “substantial identity,” as used in the context of avexitide as described herein, refers to an amino acid sequence that has at least 90% sequence identity to SEQ ID NO:1. Alternatively, percent identity can be any integer from 90% to 100%. Exemplary embodiments include at least: 90%, 91%, 92%, 93%, 94%, 95%, 96%, 97%, 98%, or 99%, as compared to SEQ ID NO:1. Each possibility represents another embodiment of the present invention.

Methods of alignment of sequences for comparison are well-known. Optimal alignment of sequences for comparison may be conducted, for example, by the local homology algorithm of Smith and Waterman, 1981, by the homology alignment algorithm of Needleman and Wunsch, 1970, by the search for similarity method of Pearson and Lipman, 1988, by computerized implementations of these algorithms (for example, BLAST), or by manual alignment and visual inspection. For sequence comparison, typically one sequence acts as a reference sequence, in this case SEQ ID NO:1, to which test sequences (for example, candidate antisense oligonucleotide sequences) are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Each possibility represents another embodiment of the present invention.

Substitutions, deletions, or additions to the amino acid sequence of SEQ ID NO:1 that add or delete a single amino acid or a small percentage of amino acids in the sequence or alterations that result in the substitution of a single amino acid or a small percentage of amino acids in the sequence with a chemically similar amino acid(s) are considered a “conservatively modified variant”. Conservative substitution tables providing functionally similar amino acids are well known in the art. Such conservatively modified variants are in addition to and do not exclude polymorphic variants, interspecies homologs, and alleles of the invention. Each possibility represents another embodiment of the present invention.

Avexitide has been well-tolerated across multiple clinical investigations conducted to date, administered as a reconstituted lyophilized formulation for continuous IV infusion and as a solution formulation for SC administration (avexitide injection). Across the Phase 1/2 investigator-initiated trials in patients with CHI or hypoglycemia after fundoplication (Protocol 2007-1-5131, Protocol 2008-10-6255, Protocol 2008-10-6256, Protocol 09-007372), the Phase 1 Eiger-sponsored PK/PD study in healthy adult volunteers (Study EIG-EXD-002), the Phase 2 Eiger-sponsored PREVENT trial in patients with post-bariatric hypoglycemia (PBH) (Study EIG-EXD-001, Craig 2021), and the Phase 2 investigator-initiated trials in patients with PBH (Salehi 2011, Salehi 2014, Craig 2017, Craig 2018, Tan 2020, Craig 2021), both formulations of avexitide have been well tolerated with no drug-related serious AEs and no discontinuations due to AEs.

In some embodiments, avexitide is administered as a formulation. In some embodiments, the formulation comprises avexitide at a concentration of 2-200 mg/mL, e.g., 5 mg/mL, 10 mg/mL, 15 mg/mL, 20 mg/mL, 25 mg/mL, 30 mg/mL, 35 mg/mL, 40 mg/mL, 45 mg/mL, 50 mg/mL, 55 mg/mL, 60 mg/mL, 65 mg/mL, 70 mg/mL, 75 mg/mL, 80 mg/mL, 85 mg/mL, 90 mg/mL, 95 mg/mL, 100 mg/mL, 105 mg/mL, 110 mg/mL, 115 mg/mL, 120 mg/mL, 125 mg/mL, 130 mg/mL, 135 mg/mL, 140 mg/mL, 145 mg/mL, 150 mg/mL, 155 mg/mL, 160 mg/mL, 165 mg/mL, 170 mg/mL, 175 mg/mL, 180 mg/mL, 185 mg/mL, 190 mg/mL, 195 mg/mL, and/or 200 mg/mL). In some embodiments, the formulation comprises avexitide at a concentration of 40 mg/mL. In some embodiments, the formulation comprises avexitide at a concentration of 100 mg/mL. In some embodiments, the formulation comprises avexitide at a concentration of 200 mg/mL.

In some embodiments, the formulation comprises avexitide or a pharmaceutically acceptable salt thereof in a physiologically acceptable buffer having a pH in the range of 5 to 6, such as pH 5.1 to pH 6.0, or pH 5.2 to pH 6.0, or pH 5.2 to pH 5.8. In some embodiments, the buffer is compatible with subcutaneous administration. In some embodiments, the physiologically acceptable buffer is a buffer that results in a liquid formulation having a pH at or about physiological pH, or within a relatively narrow pH range near physiological pH (e.g., between 5.0 and 8.0, generally above 5.1). In one embodiment, the buffered liquid formulation comprises a physiologically acceptable buffer having a pH above 5.0 and up to 6. In some embodiments, the physiologically acceptable buffer has a pH above 5.0 and up to about 5.5. In one embodiment, the physiologically acceptable buffer has a pH in the range of 5.2 to 5.8 (e.g., 5.2, 5.3, 5.4, 5.5, 5.6, 5.7, or 5.8). In one embodiment, the physiologically acceptable buffer has a pH in the range of 5.0 to 5.5 (e.g., 5.1, 5.2, 5.3, 5.4, or 5.5). In one embodiment, the physiologically acceptable buffer has a pH in the range of about 5.5 to about 6. In one embodiment, the physiologically acceptable buffer has a pH of about 5.5. Exemplary avexitide formulations are described in International Application Publication Nos. WO2018094404 and WO2020081534, which are incorporated by reference in their entireties herein for all purposes.

In some embodiments, the avexitide formulation is provided in a unit or multi-dose glass vial or ampule for administration with the use of a syringe, similar to a glucagon emergency kit. In some instances, a syringe is provided with the vial or ampule. In some instances, a syringe is not provided with the vial or ampule. In some embodiments, the avexitide formulation is provided in a pre-filled vial. In some embodiments, the avexitide formulation is provided as a single-use prefilled syringe, e.g., in a kit comprising multiple single-use prefilled syringes (e.g., 10, 20, 30, 40, 50, or 60 prefilled syringes). In some embodiments, the avexitide formulation is provided in a pen injector device. In some embodiments, the pen injector device is a glass device (e.g., a glass cartridge pen injector device). In some embodiments, the pen injector device is a single-use device. In some embodiments, the avexitide formulation is provided as an injectable solution in a single-dose tray containing a vial of an avexitide formulation as described herein, a vial connector, a syringe, and one or more needles.

### D. Routes of Administrations

In the methods of the present disclosure, avexitide may be administered by any route of administration known in the art. In some embodiments, avexitide is administered to the subject by subcutaneous, intravenous, buccal, rectal, parenteral, intraperitoneal, intradermal, intratracheal, intramuscular, and/or inhalational administration.

In some embodiments, avexitide (e.g., a formulation comprising avexitide) is administered to a subject by subcutaneous or intravenous administration (e.g., subcutaneous injection, intravenous infusion). Sites of injection, administration, or infusion, include, but are not limited to, injection in the thigh, abdomen, upper arm region, or upper buttock region.

In some embodiments, avexitide (e.g., a formulation comprising avexitide) is formulated for subcutaneous administration. In one embodiment, for example, the avexitide composition is formulated for subcutaneous administration according to a twice daily (BID) or three times daily (TID) dosing regimen.

### E. Dosing Regimen

In embodiments where avexitide is administered by injection (e.g., subcutaneous injection), the dose of avexitide administered to the subject is not particularly limited. In some embodiments, avexitide is administered subcutaneously. In some instances, avexitide is administered twice daily (BID) or three times daily (TID). In some embodiments, the avexitide is administered by intravenous infusion.

In some embodiments, avexitide is administered to the subject at any of the dosages indicated in Table 12. Each possibility represents another embodiment of the present invention.

In some embodiments, avexitide is administered to the subject at a total daily dose of 1 to 200 mg, e.g., 1 to 170 mg, 1 to 175 mg, 1 to 165 mg, 1 to 160 mg, 1 to 155 mg, 1 to 150 mg, 1 to 145 mg, 1.5 to 175 mg, 1.5 to 170 mg, 1.5 to 165 mg, 1.5 to 160 mg, 1.5 to 155 mg, 1.5 to 150 mg, 1.5 to 145 mg, 2 to 175 mg, 2 to 170 mg, 2 to 165 mg, 2 to 160 mg, 2 to 155 mg, 2 to 150 mg, 2 to 145 mg, 2.2 to 175 mg, 2.2 to 170 mg, 2.2 to 165 mg, 2.2 to 160 mg, 2.2 to 155 mg, 2.2 to 150 mg, 2.2 to 145 mg, 2.4 to 175 mg, 2.4 to 170 mg, 2.4 to 165 mg, 2.4 to 160 mg, 2.4 to 155 mg, 2.4 to 150 mg, 2.4 to 145 mg, 2.6 to 175 mg, 2.6 to 170 mg, 2.6 to 165 mg, 2.6 to 160 mg, 2.6 to 155 mg, 2.6 to 150 mg, or 2.6 to 145 mg.

In some embodiments, avexitide is administered to the subject at a total daily dose of 4 to 400 mg (e.g., 4 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, and 400 mg). In some instances, the total daily dose is 4-25 mg, 4-50 mg, 4-75 mg, 4-100 mg, 4-125 mg, 4-150 mg, 4-175 mg, 4-200 mg. In some instances, the total daily dose is 4-25 mg, 4-50 mg, 4-75 mg, 4-100 mg, 4-125 mg, 4-150 mg, 4-175 mg, 4-200 mg, 25-50 mg, 25-75 mg, 25-100 mg, 25-125 mg, 25-150 mg, 25-175 mg, 25-200 mg, 25-225 mg, 25-250 mg, 25-275 mg, 25-300 mg, 50-75 mg, 50-100 mg, 50-125 mg, 50-150 mg, 50-175 mg, 50-200 mg, 50-225 mg, 50-250 mg, 50-275 mg, 50-300 mg, 100-125 mg, 100-150 mg, 100-175 mg, 100-175 mg, 100-200 mg, 100-225 mg, 100-250 mg, 100-275 mg, 100-300 mg, 100-325 mg, 100-350 mg, 100-375 mg, 100-400 mg, 200-300 mg, 200-400 mg, 250-400 mg, or 300-400 mg.

In some embodiments, avexitide is administered to the subject at a total daily dose of 1 to 140 mg, 1 to 130 mg, 1 to 120 mg, 1 to 110 mg, 1 to 100 mg, 1 to 90 mg, 1 to 80 mg, 1 to 70 mg, 1 to 60 mg, 1 to 50 mg, 1 to 40 mg, 1 to 30 mg, 1 to 20 mg, 1 to 10 mg, 10 to 175 mg, 10 to 170 mg, 10 to 165 mg, 10 to 160 mg, 10 to 155 mg, 10 to 150 mg, 10 to 145 mg, 10 to 140 mg, 10 to 130 mg, 10 to 120 mg, 10 to 110 mg, 10 to 100 mg, 10 to 90 mg, 10 to 80 mg, 10 to 70 mg, 10 to 60 mg, 10 to 50 mg, 10 to 40 mg, 10 to 30 mg, 10 to 20 mg, 25 to 175 mg, 25 to 170 mg, 25 to 165 mg, 25 to 160 mg, 25 to 155 mg, 25 to 150 mg, 25 to 145 mg, 25 to 140 mg, 25 to 130 mg, 25 to 120 mg, 25 to 110 mg, 25 to 100 mg, 25 to 90 mg, 25 to 80 mg, 25 to 70 mg, 25 to 60 mg, 25 to 50 mg, 25 to 40 mg, 25 to 30 mg, 50 to 175 mg, 50 to 170 mg, 50 to 165 mg, 50 to 160 mg, 50 to 155 mg, 50 to 150 mg, 50 to 145 mg, 50 to 140 mg, 50 to 130 mg, 50 to 120 mg, 50 to 110 mg, 50 to 100 mg, 50 to 90 mg, 50 to 80 mg, 50 to 70 mg, 50 to 60 mg, 75 to 175 mg, 75 to 170 mg, 75 to 165 mg, 75 to 160 mg, 75 to 155 mg, 75 to 150 mg, 75 to 145 mg, 75 to 140 mg, 75 to 130 mg, 75 to 120 mg, 75 to 110 mg, 75 to 100 mg, 75 to 90 mg, 75 to 80 mg, 100 to 175 mg, 100 to 170 mg, 100 to 165 mg, 100 to 160 mg, 100 to 155 mg, 100 to 150 mg, 100 to 145 mg, 100 to 140 mg, 100 to 130 mg, 100 to 120 mg, 100 to 110 mg, 150 to 175 mg, 150 to 170 mg, 150 to 165 mg, 150 to 160 mg, 175 to 140 mg, 175 to 130 mg, 175 to 120 mg, 175 to 110 mg, 175 to 175 mg, 100 to 170 mg, 100 to 165 mg, 100 to 160 mg, 100 to 155 mg, 100 to 150 mg, 100 to 145 mg, 100 to 140 mg, 100 to 130 mg, 100 to 120 mg, 100 to 110 mg, 150 to 175 mg, 150 to 170 mg, 150 to 165 mg, or 150 to 160 mg.

In terms of lower limits, avexitide may be administered at a daily dose greater than 1 mg, e.g., greater than 1.5 mg, greater than 2 mg, greater than 2.2 mg, greater than 2.4 mg, or greater than 2.6 mg. In terms of upper limits, avexitide may be administered at a daily dose less than 175 mg, e.g., less than 170 mg, less than 165 mg, less than 160 mg, less than 155 mg, less than 150 mg, or less than 145 mg.

In some embodiments, avexitide is administered to the subject at per dose amount of 1 to 200 mg, e.g., 1 to 170 mg, 1 to 175 mg, 1 to 165 mg, 1 to 160 mg, 1 to 155 mg, 1 to 150 mg, 1 to 145 mg, 1.5 to 175 mg, 1.5 to 170 mg, 1.5 to 165 mg, 1.5 to 160 mg, 1.5 to 155 mg, 1.5 to 150 mg, 1.5 to 145 mg, 2 to 175 mg, 2 to 170 mg, 2 to 165 mg, 2 to 160 mg, 2 to 155 mg, 2 to 150 mg, 2 to 145 mg, 2.2 to 175 mg, 2.2 to 170 mg, 2.2 to 165 mg, 2.2 to 160 mg, 2.2 to 155 mg, 2.2 to 150 mg, 2.2 to 145 mg, 2.4 to 175 mg, 2.4 to 170 mg, 2.4 to 165 mg, 2.4 to 160 mg, 2.4 to 155 mg, 2.4 to 150 mg, 2.4 to 145 mg, 2.6 to 175 mg, 2.6 to 170 mg, 2.6 to 165 mg, 2.6 to 160 mg, 2.6 to 155 mg, 2.6 to 150 mg, or 2.6 to 145 mg.

In some embodiments, avexitide is administered to the subject at a total daily dose of 0.3 to 3.6 mg/kg, e.g., 0.3 to 3.4 mg/kg, 0.3 to 3.2 mg/kg, 0.3 to 3 mg/kg, 0.3 to 2.8 mg/kg, 0.3 to 2.5 mg/kg, 0.3 to 2.0 mg/kg, 0.3 to 1.5 mg/kg, 0.3 to 1.0 mg/kg, 0.3 to 0.5 mg/kg, 0.4 to 3.6 mg/kg, 0.4 to 3.4 mg/kg, 0.4 to 3.2 mg/kg, 0.4 to 3 mg/kg, 0.4 to 2.8 mg/kg, 0.5 to 3.6 mg/kg, 0.5 to 3.4 mg/kg, 0.5 to 3.2 mg/kg, 0.5 to 3 mg/kg, 0.5 to 2.8 mg/kg, 0.6 to 3.6 mg/kg, 0.6 to 3.4 mg/kg, 0.6 to 3.2 mg/kg, 0.6 to 3 mg/kg, 0.6 to 2.8 mg/kg, 0.7 to 3.6 mg/kg, 0.7 to 3.4 mg/kg, 0.7 to 3.2 mg/kg, 0.7 to 3 mg/kg, 0.7 to 2.8 mg/kg, 0.8 to 3.6 mg/kg, 0.8 to 3.4 mg/kg, 0.8 to 3.2 mg/kg, 0.8 to 3 mg/kg, 0.8 to 2.8 mg/kg.

In some embodiments, avexitide is administered to the subject at a total daily dose of 0.4 to 2.5 mg/kg, 0.4 to 2.0 mg/kg, 0.4 to 1.5 mg/kg, 0.4 to 1.0 mg/kg, 0.4 to 0.5 mg/kg, 0.5 to 2.5 mg/kg, 0.5 to 2.0 mg/kg, 0.5 to 1.5 mg/kg, 0.5 to 1.0 mg/kg, 0.6 to 2.5 mg/kg, 0.6 to 2.0 mg/kg, 0.6 to 1.5 mg/kg, 0.6 to 1.0 mg/kg, 0.7 to 2.5 mg/kg, 0.7 to 2.0 mg/kg, 0.7 to 1.5 mg/kg, 0.7 to 1.0 mg/kg, 0.8 to 2.5 mg/kg, 0.8 to 2.0 mg/kg, 0.8 to 1.5 mg/kg, 0.8 to 1.0 mg/kg, 1.0 to 3.6 mg/kg, 1.0 to 3.4 mg/kg, 1.0 to 3.2 mg/kg, 1.0 to 3 mg/kg, 1.0 to 2.8 mg/kg, 1.0 to 2.5 mg/kg, 1.0 to 2.0 mg/kg, 1.0 to 1.5 mg/kg, 1.5 to 3.6 mg/kg, 1.5 to 3.4 mg/kg, 1.5 to 3.2 mg/kg, 1.5 to 3 mg/kg, 1.5 to 2.8 mg/kg, 1.5 to 2.0 mg/kg, 2.0 to 3.6 mg/kg, 2.0 to 3.4 mg/kg, 2.0 to 3.2 mg/kg, 2.0 to 3 mg/kg, 2.0 to 2.8 mg/kg, 2.0 to 2.5 mg/kg, 2.5 to 3.6 mg/kg, 2.5 to 3.4 mg/kg, 2.5 to 3.2 mg/kg, 2.5 to 3 mg/kg, 2.5 to 2.8 mg/kg, 3.0 to 3.6 mg/kg, 3.0 to 3.4 mg/kg, 3.0 to 3.2 mg/kg, 2.8 to 3.6 mg/kg, 2.8 to 3.4 mg/kg, or 2.8 to 3.2 mg/kg.

In terms of lower limits, avexitide may be administered at a total daily dose of at least 0.3 mg/kg, e.g., at least 0.4 mg/kg, at least 0.5 mg/kg, at least 0.6 mg/kg, at least 0.7 mg/kg, or at least 0.8 mg/kg. In terms of upper limits, avexitide may be administered at a total daily dose of less than 3.6 mg/kg, e.g., less than 3.4 mg/kg, less than 3.2 mg/kg, less than 3, mg/kg, or less than 2.8 mg/kg.

In some embodiments, avexitide is administered to the subject at a total daily dose of 0.9 to 15 mg/kg, e.g., 1 mg/kg, 1.2 mg/kg, 1.4 mg/kg, 1.6 mg/kg, 1.8 mg/kg, 2 mg/kg, 2.2 mg/kg, 2.4 mg/kg, 2.6 mg/kg, 2.8 mg/kg, 3 mg/kg, 3.2 mg/kg, 3.4 mg/kg, 3.6 mg/kg, 3.8 mg/kg, 4 mg/kg, 4.2 mg/kg, 4.4 mg/kg, 4.6 mg/kg, 4.8 mg/kg, 5 mg/kg, 5.2 mg/kg, 5.4 mg/kg, 5.6 mg/kg, 5.8 mg/kg, 6 mg/kg, 6.2 mg/kg, 6.4 mg/kg, 6.6 mg/kg, 6.8 mg/kg, 7 mg/kg, 7.2 mg/kg, 7.4 mg/kg, 7.6 mg/kg, 7.8 mg/kg, 8 mg/kg, 8.2 mg/kg, 8.4 mg/kg, 8.6 mg/kg, 8.8 mg/kg, 9 mg/kg, 9.2 mg/kg, 9.4 mg/kg, 9.6 mg/kg, 9.8 mg/kg, 10 mg/kg, 10.2 mg/kg, 10.4 mg/kg, 10.6 mg/kg, 10.8 mg/kg, 11 mg/kg, 11.2 mg/kg, 11.4 mg/kg, 11.6 mg/kg, 11.8 mg/kg, 12 mg/kg, 12.2 mg/kg, 12.4 mg/kg, 12.6 mg/kg, 12.8 mg/kg, 13 mg/kg, 13.2 mg/kg, 13.4 mg/kg, 13.6 mg/kg, 13.8 mg/kg, 14 mg/kg, 14.2 mg/kg, 14.4 mg/kg, 14.6 mg/kg, 14.8 mg/kg, or 15 mg/kg.

In some embodiments, avexitide is administered to the subject at a per dose amount BID or TID of 0.3 to 3.6 mg/kg, e.g., 0.3 to 3.4 mg/kg, 0.3 to 3.2 mg/kg, 0.3 to 3 mg/kg, 0.3 to 2.8 mg/kg, 0.3 to 2.5 mg/kg, 0.3 to 2.0 mg/kg, 0.3 to 1.5 mg/kg, 0.3 to 1.0 mg/kg, 0.3 to 0.5 mg/kg, 0.4 to 3.6 mg/kg, 0.4 to 3.4 mg/kg, 0.4 to 3.2 mg/kg, 0.4 to 3 mg/kg, 0.4 to 2.8 mg/kg, 0.5 to 3.6 mg/kg, 0.5 to 3.4 mg/kg, 0.5 to 3.2 mg/kg, 0.5 to 3 mg/kg, 0.5 to 2.8 mg/kg, 0.6 to 3.6 mg/kg, 0.6 to 3.4 mg/kg, 0.6 to 3.2 mg/kg, 0.6 to 3 mg/kg, 0.6 to 2.8 mg/kg, 0.7 to 3.6 mg/kg, 0.7 to 3.4 mg/kg, 0.7 to 3.2 mg/kg, 0.7 to 3 mg/kg, 0.7 to 2.8 mg/kg, 0.8 to 3.6 mg/kg, 0.8 to 3.4 mg/kg, 0.8 to 3.2 mg/kg, 0.8 to 3 mg/kg, 0.8 to 2.8 mg/kg.

In some embodiments, avexitide is administered to the subject at a per dose amount BID or TID of 0.3 to 3.6 mg/kg, e.g., 0.3 to 3.4 mg/kg, 0.3 to 3.2 mg/kg, 0.3 to 3 mg/kg, 0.3 to 2.8 mg/kg, 0.3 to 2.5 mg/kg, 0.3 to 2.0 mg/kg, 0.3 to 1.5 mg/kg, 0.3 to 1.0 mg/kg, 0.3 to 0.5 mg/kg, 0.4 to 3.6 mg/kg, 0.4 to 3.4 mg/kg, 0.4 to 3.2 mg/kg, 0.4 to 3 mg/kg, 0.4 to 2.8 mg/kg, 0.5 to 3.6 mg/kg, 0.5 to 3.4 mg/kg, 0.5 to 3.2 mg/kg, 0.5 to 3 mg/kg, 0.5 to 2.8 mg/kg, 0.6 to 3.6 mg/kg, 0.6 to 3.4 mg/kg, 0.6 to 3.2 mg/kg, 0.6 to 3 mg/kg, 0.6 to 2.8 mg/kg, 0.7 to 3.6 mg/kg, 0.7 to 3.4 mg/kg, 0.7 to 3.2 mg/kg, 0.7 to 3 mg/kg, 0.7 to 2.8 mg/kg, 0.8 to 3.6 mg/kg, 0.8 to 3.4 mg/kg, 0.8 to 3.2 mg/kg, 0.8 to 3 mg/kg, 0.8 to 2.8 mg/kg.

In some embodiments, avexitide is administered to the subject at a per dose amount BID or TID of 0.4 to 2.5 mg/kg, 0.4 to 2.0 mg/kg, 0.4 to 1.5 mg/kg, 0.4 to 1.0 mg/kg, 0.4 to 0.5 mg/kg, 0.5 to 2.5 mg/kg, 0.5 to 2.0 mg/kg, 0.5 to 1.5 mg/kg, 0.5 to 1.0 mg/kg, 0.6 to 2.5 mg/kg, 0.6 to 2.0 mg/kg, 0.6 to 1.5 mg/kg, 0.6 to 1.0 mg/kg, 0.7 to 2.5 mg/kg, 0.7 to 2.0 mg/kg, 0.7 to 1.5 mg/kg, 0.7 to 1.0 mg/kg, 0.8 to 2.5 mg/kg, 0.8 to 2.0 mg/kg, 0.8 to 1.5 mg/kg, 0.8 to 1.0 mg/kg, 1.0 to 3.6 mg/kg, 1.0 to 3.4 mg/kg, 1.0 to 3.2 mg/kg, 1.0 to 3 mg/kg, 1.0 to 2.8 mg/kg, 1.0 to 2.5 mg/kg, 1.0 to 2.0 mg/kg, 1.0 to 1.5 mg/kg, 1.5 to 3.6 mg/kg, 1.5 to 3.4 mg/kg, 1.5 to 3.2 mg/kg, 1.5 to 3 mg/kg, 1.5 to 2.8 mg/kg, 1.5 to 2.0 mg/kg, 2.0 to 3.6 mg/kg, 2.0 to 3.4 mg/kg, 2.0 to 3.2 mg/kg, 2.0 to 3 mg/kg, 2.0 to 2.8 mg/kg, 2.0 to 2.5 mg/kg, 2.5 to 3.6 mg/kg, 2.5 to 3.4 mg/kg, 2.5 to 3.2 mg/kg, 2.5 to 3 mg/kg, 2.5 to 2.8 mg/kg, 3.0 to 3.6 mg/kg, 3.0 to 3.4 mg/kg, 3.0 to 3.2 mg/kg, 2.8 to 3.6 mg/kg, 2.8 to 3.4 mg/kg, or 2.8 to 3.2 mg/kg.

In some embodiments, avexitide is administered (e.g., subcutaneously or by intravenous or subcutaneous infusion) at a dose of 0.5 to 80 mg BID, e.g., 0.5 to 70 mg BID, 1 to 65 mg BID, 1.5 to 60 mg BID, or 1.8 to 50 mg BID. In some embodiments, avexitide is administered at a dose of 0.5 to 60 mg BID, 0.5 to 50 mg BID, 0.5 to 40 mg BID, 0.5 to 30 mg BID, 0.5 to 20 mg BID, 0.5 to 10 mg BID, 0.5 to 5 mg BID, 0.5 to 1.0 mg BID, 1.0 to 60 mg BID, 1.0 to 50 mg BID, 1.0 to 40 mg BID, 1.0 to 30 mg BID, 1.0 to 20 mg BID, 1.0 to 10 mg BID, 1.0 to 5 mg BID, 2.0 to 80 mg BID, 2.0 to 70 mg BID, 2.0 to 60 mg BID, 2.0 to 50 mg BID, 2.0 to 40 mg BID, 2.0 to 30 mg BID, 2.0 to 20 mg BID, 2.0 to 10 mg BID, 2.0 to 5 mg BID, 5.0 to 80 mg BID, 5.0 to 70 mg BID, 5.0 to 60 mg BID, 5.0 to 50 mg BID, 5.0 to 40 mg BID, 5.0 to 30 mg BID, 5.0 to 20 mg BID, 5.0 to 10 mg BID, 10 to 80 mg BID, 10 to 70 mg BID, 10 to 60 mg BID, 10 to 50 mg BID, 10 to 40 mg BID, 10 to 30 mg BID, 10 to 20 mg BID, 20 to 80 mg BID, 20 to 70 mg BID, 20 to 60 mg BID, 20 to 50 mg BID, 20 to 40 mg BID, 20 to 30 mg BID, 30 to 80 mg BID, 30 to 70 mg BID, 30 to 60 mg BID, 30 to 50 mg BID, 30 to 40 mg BID, 40 to 80 mg BID, 40 to 70 mg BID, 40 to 60 mg BID, 40 to 50 mg BID, 50 to 80 mg BID, 50 to 70 mg BID, 50 to 60 mg BID, 60 to 80 mg BID, or 60 to 70 mg BID.

In some embodiments, avexitide is administered (e.g., subcutaneously or by intravenous or subcutaneous infusion) at a dose of 50 to 200 mg BID, e.g., 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, and/or 200 mg. In some embodiments, avexitide is administered (e.g., subcutaneously or by intravenous or subcutaneous infusion) at a dose of 50 mg to 100 mg BID, 50 mg to 150 mg BID, 50 mg to 175 mg BID, 50 mg to 200 mg BID, 100 to 200 mg BID, 75 mg to 200 mg BID, or 75 mg to 150 mg BID.

In some embodiments, avexitide is administered at a dose of 0.5 mg BID, 0.8 mg BID, 1.0 mg BID, 1.2 mg BID, 2.4 mg BID, 3.6 mg BID, 4.8 mg BID, 7.5 mg BID, 8 mg BID, 10 mg BID, 15 mg BID, 20 mg BID, 25 mg BID, 30 mg BID, 35 mg BID, 40 mg BID, 45 mg BID, 50 mg BID, 60 mg BID, 70 mg BID, 80 mg BID, 100 mg BID, 125 mg BID, 150 mg BID, 175 mg BID, or 200 mg BID.

In some embodiments, the avexitide is administered at a dose of 0.9 to 3.6 mg/kg (e.g., 0.9 mg, 1 mg, 1.1 mg, 1.2 mg, 1.3 mg, 1.4 mg, 1.5 mg, 1.6 mg, 1.7 mg, 1.8 mg, 1.9 mg, 2 mg, 2.1 mg, 2.2 mg, 2.3 mg, 2.4 mg, 2.5 mg, 2.6 mg, 2.7 mg, 2.8 mg, 2.9 mg, 3 mg, 3.1 mg, 3.2 mg, 3.3 mg, 3.4 mg, 3.5 mg, and/or 3.6 mg). In some embodiments, the avexitide is administered at a dose of 1.8 to 3.6 mg/kg. In some embodiments, the avexitide is administered at a dose of 0.9 to 2.7 mg/kg. In some embodiments, the avexitide is administered at a dose of 1.8 to 2.7 mg/kg. In some embodiments, the avexitide is administered at a dose of 0.9 to 1.8 mg/kg.

In some embodiments, avexitide is administered (e.g., subcutaneously or by intravenous or subcutaneous infusion) at a dose of 0.5 to 80 mg TID, e.g., 1 to 65 mg TID, 1.5 to 60 mg TID, or 1.8 to 50 mg TID. In some embodiments, avexitide is administered at a dose of 0.5 to 60 mg TID, 0.5 to 50 mg TID, 0.5 to 40 mg TID, 0.5 to 30 mg TID, 0.5 to 20 mg TID, 0.5 to 10 mg TID, 0.5 to 5 mg TID, 0.5 to 1.0 mg TID, 1.0 to 60 mg TID, 1.0 to 50 mg TID, 1.0 to 40 mg TID, 1.0 to 30 mg TID, 1.0 to 20 mg TID, 1.0 to 10 mg TID, 1.0 to 5 mg TID, 2.0 to 80 mg TID, 2.0 to 70 mg TID, 2.0 to 60 mg TID, 2.0 to 50 mg TID, 2.0 to 40 mg TID, 2.0 to 30 mg TID, 2.0 to 20 mg TID, 2.0 to 10 mg TID, 2.0 to 5 mg TID, 5.0 to 80 mg TID, 5.0 to 70 mg TID, 5.0 to 60 mg TID, 5.0 to 50 mg TID, 5.0 to 40 mg TID, 5.0 to 30 mg TID, 5.0 to 20 mg TID, 5.0 to 10 mg TID, 10 to 80 mg TID, 10 to 70 mg TID, 10 to 60 mg TID, 10 to 50 mg TID, 10 to 40 mg TID, 10 to 30 mg TID, 10 to 20 mg TID, 20 to 80 mg TID, 20 to 70 mg TID, 20 to 60 mg TID, 20 to 50 mg TID, 20 to 40 mg BID, 20 to 30 mg BID, 30 to 80 mg BID, 30 to 70 mg BID, 30 to 60 mg TID, 30 to 50 mg TID, 30 to 40 mg TID, 40 to 80 mg TID, 40 to 70 mg TID, 40 to 60 mg TID, 40 to 50 mg TID, 50 to 80 mg TID, 50 to 70 mg TID, 50 to 60 mg TID, 60 to 80 mg TID, or 60 to 70 mg TID.

In some embodiments, avexitide is administered at a dose of 0.5 mg TID, 0.8 mg TID, 1.0 mg BID, 1.2 mg TID, 2.4 mg TID, 3.6 mg TID, 4.8 mg TID, 7.5 mg TID, 8 mg TID, 10 mg TID, 15 mg TID, 20 mg TID, 25 mg TID, 30 mg TID, 35 mg TID, 40 mg TID, 45 mg TID, or 50 mg TID.

In embodiments where avexitide is administered by infusion, the rate of infusion of avexitide to the subject is not particularly limited. In some embodiments, avexitide is administered to the subject at a rate of 100 to 1000 pmol/kg/min, e.g., 100 to 975 pmol/kg/min, 100 to 950 pmol/kg/min, 100 to 925 pmol/kg/min, 100 to 900 pmol/kg/min, 100 to 875 pmol/kg/min, 100 to 850 pmol/kg/min, 100 to 825 pmol/kg/min, 100 to 800 pmol/kg/min, 100 to 775 pmol/kg/min, 100 to 750 pmol/kg/min, 100 to 725 pmol/kg/min, 100 to 700 pmol/kg/min, 100 to 675 pmol/kg/min, 100 to 650 pmol/kg/min, 100 to 625 pmol/kg/min, 100 to 600 pmol/kg/min, 100 to 575 pmol/kg/min, 100 to 550 pmol/kg/min, 100 to 525 pmol/kg/min, or 100 to 500 pmol/kg/min.

In some embodiments, avexitide is administered to the subject at a rate of 125 to 1000 pmol/kg/min, 125 to 975 pmol/kg/min, 125 to 950 pmol/kg/min, 125 to 925 pmol/kg/min, 125 to 900 pmol/kg/min, 125 to 875 pmol/kg/min, 125 to 850 pmol/kg/min, 125 to 825 pmol/kg/min, 125 to 800 pmol/kg/min, 125 to 775 pmol/kg/min, 125 to 750 pmol/kg/min, 125 to 725 pmol/kg/min, 125 to 700 pmol/kg/min, 125 to 675 pmol/kg/min, 125 to 650 pmol/kg/min, 125 to 625 pmol/kg/min, 125 to 600 pmol/kg/min, 125 to 575 pmol/kg/min, 125 to 550 pmol/kg/min, 125 to 525 pmol/kg/min, or 125 to 500 pmol/kg/min.

In some embodiments, avexitide is administered to the subject at a rate of 150 to 1000 pmol/kg/min, 150 to 975 pmol/kg/min, 150 to 950 pmol/kg/min, 150 to 925 pmol/kg/min, 150 to 900 pmol/kg/min, 150 to 875 pmol/kg/min, 150 to 850 pmol/kg/min, 150 to 825 pmol/kg/min, 150 to 800 pmol/kg/min, 150 to 775 pmol/kg/min, 150 to 750 pmol/kg/min, 150 to 725 pmol/kg/min, 150 to 700 pmol/kg/min, 150 to 675 pmol/kg/min, 150 to 650 pmol/kg/min, 150 to 625 pmol/kg/min, 150 to 600 pmol/kg/min, 150 to 575 pmol/kg/min, 150 to 550 pmol/kg/min, 150 to 525 pmol/kg/min, or 150 to 500 pmol/kg/min.

In some embodiments, avexitide is administered to the subject at a rate of 175 to 1000 pmol/kg/min, 175 to 975 pmol/kg/min, 175 to 950 pmol/kg/min, 175 to 925 pmol/kg/min, 175 to 900 pmol/kg/min, 175 to 875 pmol/kg/min, 175 to 850 pmol/kg/min, 175 to 825 pmol/kg/min, 175 to 800 pmol/kg/min, 175 to 775 pmol/kg/min, 175 to 750 pmol/kg/min, 175 to 725 pmol/kg/min, 175 to 700 pmol/kg/min, 175 to 675 pmol/kg/min, 175 to 650 pmol/kg/min, 175 to 625 pmol/kg/min, 175 to 600 pmol/kg/min, 175 to 575 pmol/kg/min, 175 to 550 pmol/kg/min, 175 to 525 pmol/kg/min, or 175 to 500 pmol/kg/min.

In some embodiments, avexitide is administered to the subject at a rate of 200 to 1000 pmol/kg/min, 200 to 975 pmol/kg/min, 200 to 950 pmol/kg/min, 200 to 925 pmol/kg/min, 200 to 900 pmol/kg/min, 200 to 875 pmol/kg/min, 200 to 850 pmol/kg/min, 200 to 825 pmol/kg/min, 200 to 800 pmol/kg/min, 200 to 775 pmol/kg/min, 200 to 750 pmol/kg/min, 200 to 725 pmol/kg/min, 200 to 700 pmol/kg/min, 200 to 675 pmol/kg/min, 200 to 650 pmol/kg/min, 200 to 625 pmol/kg/min, 200 to 600 pmol/kg/min, 200 to 575 pmol/kg/min, 200 to 550 pmol/kg/min, 200 to 525 pmol/kg/min, 200 to 500 pmol/kg/min.

In some embodiments, avexitide is administered to the subject at a rate of 300 to 1000 pmol/kg/min, 350 to 1000 pmol/kg/min, 400 to 1000 pmol/kg/min, 450 to 1000 pmol/kg/min, 500 to 1000 pmol/kg/min, 550 to 1000 pmol/kg/min, 600 to 1000 pmol/kg/min, 650 to 1000 pmol/kg/min, 700 to 1000 pmol/kg/min, 750 to 1000 pmol/kg/min, 800 to 1000 pmol/kg/min, 850 to 1000 pmol/kg/min, 900 to 1000 pmol/kg/min, 950 to 1000 pmol/kg/min, 300 to 900 pmol/kg/min, 300 to 800 pmol/kg/min, 300 to 700 pmol/kg/min, 300 to 600 pmol/kg/min, 300 to 1000 pmol/kg/min, 400 to 900 pmol/kg/min, 400 to 800 pmol/kg/min, 400 to 700 pmol/kg/min, 400 to 600 pmol/kg/min, 400 to 1000 pmol/kg/min, 500 to 900 pmol/kg/min, 500 to 800 pmol/kg/min, 500 to 700 pmol/kg/min, 500 to 600 pmol/kg/min, 500 to 1000 pmol/kg/min, 600 to 900 pmol/kg/min, 600 to 800 pmol/kg/min, or 600 to 700 pmol/kg/min

In terms of lower limits, avexitide may be administered at a rate greater than 100 pmol/kg/min, e.g., greater than 125 pmol/kg/min, greater than 150 pmol/kg/min, greater than 175 pmol/kg/min, or greater than 200 pmol/kg/min. In terms of upper limits, avexitide may be administered at a rate less than 1000 pmol/kg/min, less than 975 pmol/kg/min, less than 950 pmol/kg/min, less than 925 pmol/kg/min, less than 900 pmol/kg/min, less than 875 pmol/kg/min, less than 850 pmol/kg/min, less than 825 pmol/kg/min, less than 800 pmol/kg/min, less than 775 pmol/kg/min, less than 750 pmol/kg/min, less than 725 pmol/kg/min, less than 700 pmol/kg/min, less than 675 pmol/kg/min, less than 650 pmol/kg/min, less than 625 pmol/kg/min, less than 600 pmol/kg/min, less than 575 pmol/kg/min, less than 550 pmol/kg/min, less than 525 pmol/kg/min, or less than 500 pmol/kg/min.

In some embodiments, the subject is administered avexitide by infusion (e.g., intravenous infusion) for 6 to 48 hours, e.g., 6 to 42 hours, 6 to 36 hours, 6 to 30 hours, 6 to 24 hours, 6 to 18 hours, 6 to 12 hours, 7 to 48 hours, 7 to 42 hours, 7 to 36 hours, 7 to 30 hours, 7 to 24 hours, 7 to 18 hours, 7 to 12 hours, 8 to 48 hours, 8 to 42 hours, 8 to 36 hours, 8 to 30 hours, 8 to 24 hours, 8 to 18 hours, 8 to 12 hours, 9 to 48 hours, 9 to 42 hours, 9 to 36 hours, 9 to 30 hours, 9 to 24 hours, 9 to 18 hours, 9 to 12 hours, 10 to 48 hours, 10 to 42 hours, 10 to 36 hours, 10 to 30 hours, 10 to 24 hours, 10 to 18 hours, or 10 to 12 hours. In terms of lower limits, the subject may be administered avexitide for at least 6 hours, e.g., at least 7 hours, at least 8 hours, at least 9 hours, or at least 10 hours. In terms of upper limits, the subject may be administered avexitide for less than 48 hours, e.g., less than 42 hours, less than 36 hours, less than 30 hours, or less than 24 hours.

In some embodiments, avexitide is administered by subcutaneous injection. In some embodiments, avexitide is administered in a total volume of injectate of 0.05 to 1 mL (e.g., 0.05 mL, 0.1 mL, 0.15 mL, 0.2 mL, 0.25 mL, 0.3 mL, 0.35 mL, 0.4 mL, 0.45 mL, 0.5 mL, 0.55 mL, 0.6 mL, 0.65 mL, 0.7 mL, 0.75 mL, 0.8 mL, 0.85 mL, 0.9 mL, 0.95 mL, and/or 1 mL). In some embodiments, each dose is administered in a total volume of 0.05 ml to 0.1 ml, with many subjects administering an injection volume ranging from 0.25-1.5 ml, or from 0.5-1 ml, or from 0.7-1 ml.

In some embodiments, avexitide treatment is continued for less than 1 week. In some embodiments, avexitide treatment is continued for at least 1 week. For example, avexitide treatment can be continued for at least 1 day, at least 2 days, at least 3 days, at least 4 days, at least 5 days, at least 6 days, at least 1 week, at least at least 2 weeks, at least 4 weeks, at least 6 weeks, at least 8 weeks, at least 10 weeks, or at least 12 weeks. In some embodiments, avexitide treatment is continued for at least 1 year. In some embodiments, avexitide treatment is continued for at least 24 months. In some embodiments, avexitide treatment is continued for 1, 2, 3, 4, 5, 6, 7, 8, 9, 10, 11, 12, 13, 14, 15, 16, 17, 18 years, or longer. In some embodiments, avexitide treatment is continued until the subject can maintain euglycemia without administration further administration of avexitide.

As discussed above, in certain embodiments, the subject is receiving continuous intravenous glucose infusion (e.g., through a central or peripheral line). Such subjects are typically neonates or infants, as discussed above. In addition to the dosing features discussed in the embodiments above, certain dosing features of these methods are further recited below.

In some embodiments, avexitide is administered to such patients at a total daily dose of 1 mg to 175 mg (e.g., 1 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, or 175 mg). In some embodiments, avexitide is administered to such patients at a total daily dose of 2 mg to 12 mg, such as, for example, 2.7 mg to 10.8 mg, such as for example, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 10.5 mg, 11 mg, 11.5 mg, or 12 mg. In some embodiments, the avexitide is administered to such patients at a dose of 0.5 mg to 80 mg BID or TID.

In some instances, avexitide is administered to such patients subcutaneously at a dose of at least 0.6 mg/kg TID. In other instances, avexitide is administered to such patients subcutaneously at 0.6 to 3.6 mg/kg TID (e.g., 0.6 mg/kg TID, 0.8 mg/kg TID, 1 mg/kg TID, 1.2 mg/kg TID, 1.4 mg/kg TID, 1.6 mg/kg TID, 1.8 mg/kg TID, 2 mg/kg TID, 2.2 mg/kg TID, 2.4 mg/kg TID, 2.6 mg/kg TID, 2.8 mg/kg TID, 3 mg/kg TID, 3.2 mg/kg TID, 3.4 mg/kg TID, 3.6 mg/kg TID, and/or 3.8 mg/kg TID). In other instances, avexitide is administered to such patients subcutaneously at 0.9 to 3.6 mg/kg TID. In some embodiments, avexitide is administered to such patients subcutaneously at a dose of at least 1.2 mg/kg BID. In some instances, the avexitide is administered to such patients by infusion at a rate from 100-1000 pmol/kg/min.

In some instances, the avexitide is administered to such patients for at least 6 hours. In some instances, the avexitide is administered to such patients for less than 48 hours. In some instances, the avexitide is administered to such patients for at least 48 hours. In some instances, a dose of avexitide is administered to such patients for at least 72 hours. In some instances, the dose is escalated after 24 hours within a 72 hour period if the mean (8-hour) GIR indicates that GIR #0.

As discussed above, in certain embodiments, the subject is administered a total daily dose of 4 mg to 400 mg of avexitide. Such subjects are typically older infants and children, as discussed above. In some embodiments, the dosage of avexitide is administered to the subject based on the subject's body weight. In addition to the dosing features in the embodiments above, certain dosing features of these methods are further recited below.

In some embodiments, avexitide is administered subcutaneously. In some instances, avexitide is administered twice daily (BID). In some embodiments, avexitide is administered at a total daily dose of 4 mg to 400 mg. In some embodiments, avexitide is administered at a total daily dose of 4 mg, 14 mg, 24 mg, 34 mg, 44 mg, 54 mg, 64 mg, 74 mg, 84 mg, 94 mg, 104 mg, 114 mg, 124 mg, 134 mg, 144 mg, 154 mg, 164 mg, 174 mg, 184 mg, 194 mg, 204 mg, 214 mg, 224 mg, 234 mg, 244 mg, 254 mg, 264 mg, 274 mg, 284 mg, 294 mg, 304 mg, 314 mg, 324 mg, 334 mg, 344 mg, 354 mg, 364 mg, 374 mg, 384 mg, 394 mg, and/or 400 mg.

In some embodiments, avexitide is administered at a total daily dose of 4 mg to 210 mg (e.g., 4 mg, 14 mg, 24 mg, 34 mg, 44 mg, 54 mg, 64 mg, 74 mg, 84 mg, 94 mg, 104 mg, 114 mg, 124 mg, 134 mg, 144 mg, 154 mg, 164 mg, 174 mg, 184 mg, 194 mg, 204 mg, or 210 mg) to a subject that has a body weight of 56 kg or less; for example, in some instances, the subject has a body weight of 2.3 to 56 kg. In some embodiments, avexitide is administered 0.9 to 3.6 mg/kg BID for a subject that has a body weight of 56 kg or less; for example, in some instances, the subject has a body weight of 2.3 to 56 kg. In some embodiments, avexitide is administered at a total daily dose of 100 mg to 400 mg (e.g., 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, and/or 400 mg) to a subject that has a body weight of more than 56 kg.

In some embodiments, avexitide is administered (e.g., subcutaneously or by intravenous or subcutaneous infusion) at a dose of 50 to 100 mg BID, 50 to 150 mg BID, or 50 to 200 mg BID, e.g., 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, and/or 200 mg.

In some embodiments, avexitide is administered in a total volume of injectate is 0.05 to 1 mL. In some embodiments, avexitide is administered by intravenous infusion. In some embodiments, avexitide is administered at a rate from 100-1000 pmol/kg/min. In some embodiments, avexitide is administered for at least 6 hours.

The dosage ranges described above are exemplary adult doses, neonate and child doses, and may vary depending upon the age and weight of the patient as would be known by those skilled in the pharmaceutical arts. It will be appreciated that in some embodiments, dosage may be increased or decreased during the course of treatment. For example, some physicians may desire to treat with a low or initiating (starting) dose, escalate to an increased dose if the initiating dose does not provide sufficient therapeutic benefit, and maintain the initiating dose if the initiating dose provides sufficient therapeutic benefit.

In some embodiments, dose escalation can be performed until mean GIR (e.g., over a period of 6 hours, 8 hours, 12 hours, or 24 hours) is zero or close to zero in a subject. In some embodiments, avexitide is administered as a priming or bolus dose at 0.9 to 3.6 mg/kg. In some embodiments, avexitide can be administered in one or more additional doses, optionally escalating doses, until the mean GIR is zero or close to zero in the subject. In some embodiments, after the priming or bolus dose, avexitide can be administered at a first maintenance dose of 0.9-1.8 mg/kg TID. In some embodiments, if mean GIR is not zero or close to zero after the first maintenance dose, avexitide can be administered at a second maintenance dose of 1.8-2.7 mg/kg TID. In some embodiments, if mean GIR is not zero or close to zero after the second maintenance dose, avexitide can be administered at a second maintenance dose of 3.6 mg/kg TID. In some embodiments, if the subject is less than 14 days old, the priming or bolus dose can be 0.9-1.8 mg/kg, the first maintenance dose can be 0.9-1.8 mg/kg TID, the second maintenance dose (if GIR is not zero) can be 1.8-2.7 mg/kg TID, and the third maintenance dose (if GIR is not zero) can be 3.6 mg/kg TID. In some embodiments, if the subject is at least 14 days old, the priming or bolus dose can be 2.7-3.6 mg/kg, the first maintenance dose can be 1.8 mg/kg TID, the second maintenance dose (if GIR is not zero) can be 2.7 mg/kg TID, and the third maintenance dose (if euglycemia is not achieved) can be 3.6 mg/kg TID.

In some embodiments, for subjects that are 56 kg or less, dosing can be administered starting at 0.9 mg/kg BID or TID and increased if the subject experiences hypoglycemia. In some instances, a first increased dose of 1.8 mg/kg BID or TID can be administered. In some embodiments, if the subject experiences hypoglycemia, a second increased dose of 2.7 kg/mg BID or TID can be administered. In some embodiments, if the subject experiences hypoglycemia, a third increased dose of 3.6 kg/mg BID or TID can be administered. In some embodiments, for subjects that are greater than 56 kg, avexitide can be initially administered at 50 mg BID. In some embodiments, if the subject experiences hypoglycemia, dosing can be increased to first to 100 mg BID, then to 150 mg BID (if the subject experiences hypoglycemia), and then to 200 mg BID (if the subject experiences hypoglycemia).

Dose escalation can proceed after 24 hours, 48 hours, 72 hours, 96 hours, or 120 hours on a given dose.

The avexitide can be administered at specific points in the day or schedule of a subject, e.g., morning, afternoon, evening, night, before or during or after meals, before bedtime, etc. In some embodiments, avexitide is administered about once every 12 hours. In some embodiments BID doses are administered at about 12 hour intervals (e.g., 7 a.m. and 7 p.m.). However, shorter (e.g., 8 a.m. and 6 p.m.) or longer (e.g., 7 a.m. and 10 p.m.) intervals between administrations are possible. In some embodiments, the administrations are at least about 4 hours, 6 hours, 7 hours, 8 hours, 9 hours, 10 hours or 11 hours apart. Preferably the administrations are not more than about 15 hours apart.

In some embodiments, avexitide is administered (e.g., subcutaneously administered) twice daily (BID) within about 60 minutes prior to morning and evening meals (or prior to the two main meals of the day). In some embodiments, avexitide is administered at least about 60 minutes prior to a meal (e.g., at least 60 minutes prior to a morning meal and/or at least 60 minutes prior to an evening meal). In some embodiments, the administrations prior to the morning and evening meals (or prior to the two main meals of the day) are at least about 6 hours apart. In some embodiments, the administration of the formulation is not timed to meals.

In some embodiments, avexitide is administered (e.g., subcutaneously administered) three times daily (TID) within about 60 minutes of a meal (e.g., prior to the three meals in the day). In some embodiments, the administrations are spaced at least 1, 2, 3, 4, 5, 6, 7, or 8 hours apart. In some embodiments, at least two adjacent administrations are at least about 2, 3, 4, 5, 6, 7, 8, 9, 10, or 12 hours apart. In some embodiments, the administration of the formulation is not timed to meals. In some embodiments, administration is based on the blood glucose level of the subject.

### III. Exemplary Embodiments

As used below, any reference to a series of embodiments is to be understood as a reference to each of those embodiments disjunctively (e.g., “Embodiments 1-4” is to be understood as “Embodiments 1, 2, 3, or 4”).

Embodiment 1 is a method of reducing a glucose infusion rate (GIR) for a subject having congenital hyperinsulinism (CHI), the method comprising administering a therapeutically effective amount of avexitide to the subject.

Embodiment 2 is the method of embodiment(s) 1, wherein the method reduces the GIR of the subject to ≤10 mg/kg/min.

Embodiment 3 is the method of any one of embodiment(s) 1-2, wherein the method reduces the GIR of the subject by 5% to 100%.

Embodiment 4 is the method of any one of embodiment(s) 1-3, wherein the method reduces the GIR of the subject by 15% to 60%.

Embodiment 5 is the method of any one of embodiment(s) 1-4, wherein the method reduces the GIR to 0.

Embodiment 6 is the method of any one of embodiment(s) 1-5, wherein the subject has a baseline GIR of 1 to 40 mg/kg/min.

Embodiment 7 is the method of any one of embodiment(s) 1-6, wherein the avexitide is administered by intravenous infusion.

Embodiment 8 is the method of embodiment 7, wherein the avexitide is administered at a rate from 100-1000 pmol/kg/min.

Embodiment 9 is the method of any one of embodiment(s) 7-8, wherein the avexitide is administered for at least 6 hours.

Embodiment 10 is the method of any one of embodiment(s) 7-9, wherein the avexitide is administered for less than 48 hours.

Embodiment 11 is the method of any one of embodiment(s) 1-10, wherein the avexitide is administered subcutaneously.

Embodiment 12 is the method of embodiment 11, wherein the avexitide is administered twice daily (BID) or three times daily (TID).

Embodiment 13 is the method of any one of embodiment(s) 11-12, wherein the avexitide is administered at a total daily dose from 1 mg to 175 mg.

Embodiment 14 is the method of any one of embodiment(s) 11-13, wherein the avexitide is administered at a dose of 0.5 to 80 mg BID or TID.

Embodiment 15 is the method of any one of embodiment(s) 1-14, wherein the subject is less than 18 years old.

Embodiment 16 is the method of any one of embodiment(s) 15, wherein the subject is from 1 year old to 12 years old.

Embodiment 17 is the method of embodiment 15, wherein the subject is less than 1 year old.

Embodiment 18 is the method of any one of embodiment(s) 1-17, wherein the CHI is associated with a genetic abnormality, a mutation, or a syndrome.

Embodiment 19 is the method of embodiment 18, wherein the genetic abnormality, mutation, or syndrome is a mutation in a gene encoding a sulfonylurea receptor (SUR-1), a gene encoding glucokinase (GCK), a gene encoding glutamate dehydrogenase (GLUD-1), a gene encoding mitochondrial enzyme short-chain 3-hydroxyacyl-CoA dehydrogenase (HADHSC), and/or any of the genes listed in Table 1.

Embodiment 20 is a method of treating congenital hyperinsulinism (CHI) in a subject, the method comprising subcutaneously administering a therapeutically effective amount of avexitide to the subject.

Embodiment 21 is the method of embodiment 20, wherein the method reduces the GIR of the subject.

Embodiment 22 is the method of embodiment 21, wherein the method reduces the GIR of the subject to ≤10 mg/kg/min.

Embodiment 23 is the method of any one of embodiment(s) 21-22, wherein the method reduces the GIR of the subject by 5% to 100%.

Embodiment 24 is the method of any one of embodiment(s) 21-23, wherein the method reduces the GIR of the subject by 15% to 60%.

Embodiment 25 is the method of any one of embodiment(s) 21-24, wherein the method reduces the GIR to 0.

Embodiment 26 is the method of any one of embodiment(s) 20-25, wherein the subject has a baseline GIR of 1 to 40 mg/kg/min.

Embodiment 27 is the method of any one of embodiment(s) 20-26, wherein the avexitide is administered twice daily (BID) or three times daily (TID).

Embodiment 28 is the method of any one of embodiment(s) 20-27, wherein the avexitide is administered at a total daily dose from 1 mg to 175 mg.

Embodiment 29 is the method of any one of embodiment(s) 20-28, wherein the avexitide is administered at a dose of 0.5 to 80 mg BID or TID.

Embodiment 30 is the method of any one of embodiment(s) 20-29, wherein the subject is less than 18 years old.

Embodiment 31 is the method of embodiment 30, wherein the subject is from 1 year old to 12 years old.

Embodiment 32 is the method of embodiment 30, wherein the subject is less than 1 year old.

Embodiment 33 is the method of any one of embodiment(s) 20-32, wherein the CHI is associated with a genetic abnormality, a mutation, or a syndrome.

Embodiment 34 is the method of embodiment(s) 33, wherein the genetic abnormality, mutation, or syndrome is a mutation in a gene encoding a sulfonylurea receptor (SUR-1), a gene encoding glucokinase (GCK), a gene encoding glutamate dehydrogenase (GLUD-1), a gene encoding mitochondrial enzyme short-chain 3-hydroxyacyl-CoA dehydrogenase (HADHSC), and/or any of the genes listed in Table 1.

Embodiment 36 is a method of treating a subject with congenital hyperinsulinism (CHI), wherein the subject is an infant receiving continuous intravenous glucose infusion at a GIR, the method comprising administering, optionally subcutaneously administering, a therapeutically effective amount of avexitide to the subject.

Embodiment 37 is a method of reducing a glucose infusion rate (GIR) for a subject having congenital hyperinsulinism (CHI), the method comprising administering a therapeutically effective amount of avexitide to the subject.

Embodiment 38 is the method of any one of Embodiments(s) 36-37, wherein the subject requires continuous glucose infusion.

Embodiment 39 is the method of any one of Embodiments(s) 36-38, wherein the subject has a baseline GIR of 1 to 50 mg/kg/min.

Embodiment 40 is the method of any one of Embodiments(s) 36-39, wherein the avexitide is administered subcutaneously.

Embodiment 41 is the method of any one of Embodiments(s) 36-40, wherein the avexitide is administered twice daily (BID) or three times daily (TID).

Embodiment 42 is the method of any one of Embodiments(s) 36-41, wherein the avexitide is administered at a total daily dose from 1 mg to 175 mg.

Embodiment 43 is the method of any one of Embodiments(s) 36-42, wherein the avexitide is administered at a total daily dose from 2.7 to 10.8 mg.

Embodiment 44 is the method of any one of Embodiments(s) 36-43, wherein the avexitide is administered at a dose of 0.5 to 80 mg BID or TID.

Embodiment 45 is the method of any one of Embodiments(s) 36-44, wherein the avexitide is administered at a dose of 0.9 to 2.7 mg/kg TID, optionally for at least 72 hours.

Embodiment 46 is the method of any one of Embodiments(s) 36-45, wherein the avexitide is administered at a dose of 0.9 to 3.6 mg/kg TID, optionally for at least 72 hours.

Embodiment 47 is the method of any one of Embodiments(s) 36-46, wherein the avexitide is administered by intravenous infusion.

Embodiment 48 is the method of Embodiments(s) 47, wherein the avexitide is administered at a rate from 100-1000 pmol/kg/min.

Embodiment 49 is the method of any one of Embodiments(s) 47-48, wherein the avexitide is administered for at least 6 hours.

Embodiment 50 is the method of any one of Embodiments(s) 36-49, wherein treatment is continued for at least 24 months.

Embodiment 51 is the method of any one of Embodiments(s) 36-50, wherein the subject is less than 2 years old.

Embodiment 52 is the method of any one of Embodiments(s) 36-51, wherein the subject is less than 1 year old.

Embodiment 53 is the method of any one of Embodiments(s) 36-52, wherein the subject is a neonate.

Embodiment 54 is the method of any one of Embodiments(s) 36-53, wherein the subject is less than 14 days old.

Embodiment 55 is the method of any one of Embodiments(s) 36-54, wherein the subject is 14 to 364 days old.

Embodiment 56 is the method of any one of Embodiments(s) 36-55, wherein the administering reduces the GIR required to maintain euglycemia of the subject.

Embodiment 57 is the method of any one of Embodiments(s) 36-56, wherein the administering reduces the GIR of the subject to less than or equal to 10 mg/kg/min.

Embodiment 58 is the method of any one of Embodiments(s) 36-57, wherein the administering reduces the GIR of the subject by 5% to 100%.

Embodiment 59 is the method of any one of Embodiments(s) 36-58, wherein the administering reduces the GIR of the subject by 15% to 60%.

Embodiment 60 is the method of any one of Embodiments(s) 36-59, wherein the administering reduces the GIR to 0. The method of example(s) 1 or 2, wherein the administering permits stopping continuous glucose infusion for the subject.

Embodiment 61 is the method of any one of Embodiments(s) 36-60, wherein the administering reduces at least one of the total amount or volume of glucose required to be administered to the subject to maintain euglycemia or the need for IV carbohydrate rescue of the subject.

Embodiment 62 is the method of any one of Embodiments(s) 36-61, wherein the administering reduces at least one of hypoglycemia event rate or clinically important hypoglycemia event rate.

Embodiment 62 is the method of any one of Embodiments(s) 36-61, wherein the administering reduces time to central or peripheral line removal, and optionally, time to discharge readiness.

Embodiment 63 is the method of any one of Embodiments(s) 36-62, wherein the administering permits avoidance of performing a pancreatectomy on the subject.

Embodiment 64 is a method of treating congenital hyperinsulinism (CHI) in a subject, the method comprising subcutaneously administering a total daily dose of 4 mg to 400 mg of avexitide to the subject. The method of example(s) 39, wherein the subject has a body weight of 56 kg or less, optionally 2.3 to 56 kg.

Embodiment 65 is the method of Embodiments 64, wherein the avexitide is administered at a total daily dose from 4 mg to 210 mg.

Embodiment 66 is the method of any one of Embodiments(s) 64-65, wherein the avexitide is administered at 0.9 to 3.6 mg/kg BID.

Embodiment 67 is the method of any one of Embodiments(s) 64-66, wherein the subject has been administered avexitide for at least 10 weeks, and wherein the subject is subsequently administered avexitide at a dose of 1.8 kg/mg to 3.6 mg/kg BID for at least 24 months.

Embodiment 68 is the method of Embodiments 67, wherein the subject has a body weight of more than 56 kg.

Embodiment 69 is the method of any one of Embodiments 67-68, wherein the avexitide is administered at a total daily dose from 100 to 400 mg.

Embodiment 70 is the method of any one of Embodiments 67-69, wherein the avexitide is administered at 50 mg to 200 mg BID.

Embodiment 71 is the method of any one of Embodiments 67-70, wherein the subject has been administered avexitide for at least 10 weeks, and wherein the subject is subsequently administered avexitide at a dose of 100 mg to 200 mg BID for at least 24 months.

Embodiment 72 is the method of any one of Embodiments 67-71, wherein the avexitide is administered twice daily (BID).

Embodiment 73 is a method of reducing the hypoglycemia event rate for a subject having congenital hyperinsulinism (CHI), the method comprising administering therapeutically effective dose of avexitide to the subject, wherein the therapeutically effective dose is a total daily dose of 4 mg to 210 mg if the subject weighs 56 kg or less, optionally 2.3 to 56 kg, and is a total daily dose of 100 mg to 400 mg if the subject weighs more than 56 kg.

Embodiment 74 is the method of Embodiments 73, wherein the avexitide is administered subcutaneously.

Embodiment 75 is the method of Embodiments 74, wherein the avexitide is administered twice daily (BID).

Embodiment 76 is the method of any one of Embodiments 74-75, wherein the subject has a body weight of 56 kg or less, (optionally 2.3 to 56 kg), and wherein the avexitide is administered at 0.9 to 3.6 mg/kg BID.

Embodiment 77 is the method of any one of Embodiments 74-76, wherein the subject has a body weight of more than 56 kg, and wherein the avexitide is administered at 50 mg to 200 mg BID.

Embodiment 78 is the method of any one of Embodiments 64-76, wherein the total volume of injectate is 0.05 to 1 mL.

Embodiment 79 is the method of Embodiments 73, wherein the avexitide is administered by intravenous infusion.

Embodiment 80 is the method of Embodiments 79, wherein the avexitide is administered at a rate from 100-1000 pmol/kg/min.

Embodiment 81 is the method of any one of Embodiments 79-80, wherein the avexitide is administered for at least 6 hours.

Embodiment 82 is the method of any one of Embodiments 63-81, wherein treatment is continued for at least 24 months.

Embodiment 83 is the method of any one of Embodiments 63-82, wherein the subject is less than 18 years old.

Embodiment 84 is the method of any one of Embodiments 63-83, wherein the subject is 1 year old to 12 years old.

Embodiment 85 is the method of any one of Embodiments 63-84, wherein the subject is an infant.

Embodiment 86 is the method of any one of Embodiments 63-85, wherein the subject is a neonatal subject.

Embodiment 87 is the method of any one of Embodiments 63-86, wherein the subject is 18 years old or older.

Embodiment 88 is the method of any one of Embodiments 63-87, wherein the subject has documented uncontrolled hypoglycemia and, optionally, wherein the subject does not require continuous glucose infusion.

Embodiment 89 is the method of any one of Embodiments 63-88, wherein treatment thereby reduces in the subject at least one of hypoglycemia event rate, clinically important hypoglycemia event rate, or severe hypoglycemia event rate.

Embodiment 90 is the method of any one of Embodiments 63-89, wherein treatment thereby reduces in the subject at least one of TBR Level 1, TBR Level 1 Nocturnal, TBR Level 2, or TIR.

Embodiment 91 is the method of any one of Embodiments 63-90, wherein treatment thereby permits reduction of at least one of total carbohydrates administered via oral route, nasogastric tube, or gastrostomy tube per week to treat or prevent hypoglycemia events.

Embodiment 92 is the method of any one of Embodiments 63-91, wherein treatment thereby permits reduction of total nightly carbohydrates administered.

Embodiment 93 is the method of any one of Embodiments 63-92, wherein treatment thereby permits removal of a nasogastric tube or gastrostomy tube from the subject.

Embodiment 94 is the method of any of the preceding embodiments, wherein the avexitide is administered at a dose set forth in Table 12.

Embodiment 95 is the method of any of the preceding embodiments, wherein the subject is unresponsive to medical treatment with diazoxide or octreotide.

Embodiment 96 is the method of any of the preceding embodiments, wherein the subject is responsive to medical treatment with diazoxide.

Embodiment 97 is the method of any of the preceding embodiments, wherein the CHI is associated with a genetic abnormality, a mutation, or a syndrome.

Embodiment 98 is the method of any of Embodiment 97, wherein the genetic abnormality, mutation, or syndrome is a mutation in a gene encoding a sulfonylurea receptor (SUR-1), a gene encoding glucokinase (GCK), a gene encoding glutamate dehydrogenase (GLUD-1), a gene encoding mitochondrial enzyme short-chain 3-hydroxyacyl-CoA dehydrogenase (HADHSC), and/or any of the genes listed in Table 1.

Embodiment 99 is the method of any of the preceding embodiments, wherein the subject during hypoglycemia has at least one of hyperinsulinemia, hypofattyacidemia, hypoketonemia, or a glycemic response.

Embodiment 10 is the method of any of the preceding embodiments, wherein the reduction in GIR is measured in comparison to at least one of treatment of the subject with placebo or a baseline GIR of the subject.

### EXAMPLES

The following examples are provided to illustrate, but not to limit, the claimed invention.

### Example 1: Effect of Avexitide on Glucose Requirements to Maintain Euglycemia in Infants with Diazoxide-Unresponsive K_{ATP} CHI

### A. Methods

This was a placebo-controlled, randomized, crossover study to evaluate the effect of avexitide on glucose requirements to maintain euglycemia in infants with diazoxide-unresponsive KATP CHI (ClinicalTrials.gov Identifier: NCT00835328). The use of synthetic avexitide in this study was approved under the U.S. Food and Drug Administration IND 76612. The primary aim of this study was to evaluate the effect of avexitide on glucose requirements to maintain euglycemia in pediatric patients with congenital hyperinsulinism (CHI) who have failed medical therapy. The secondary aims are to determine the therapeutic plasma levels, plasma half-life and pharmacokinetics of avexitide (Exendin (9-39)) during a 9-hour intravenous infusion.

Where not otherwise specified herein, avexitide acetate is used.

Study Design. An open-label, two-period, two-treatment crossover study design with a dose-escalation component was implemented. Successive cohorts of patients (up to 5 participants/cohort) each received a fixed dose of avexitide infusion and normal saline vehicle on two separate days in random order. The protocol specifies 0.02 mg/kg/hr, via continuous intravenous infusion, administered over 9-hours for the first cohort. The volume of saline to be infused will be calculated to match the volume of avexitide. Successive cohorts will be given doses that are increased in up to 1/2 log increments. The study was designed to test the hypothesis that antagonism of the GLP-1 receptor by avexitide would increase fasting blood glucose levels, prevent protein-induced hypoglycemia, and decrease glucose requirement to maintain euglycemia in infants with CHI.

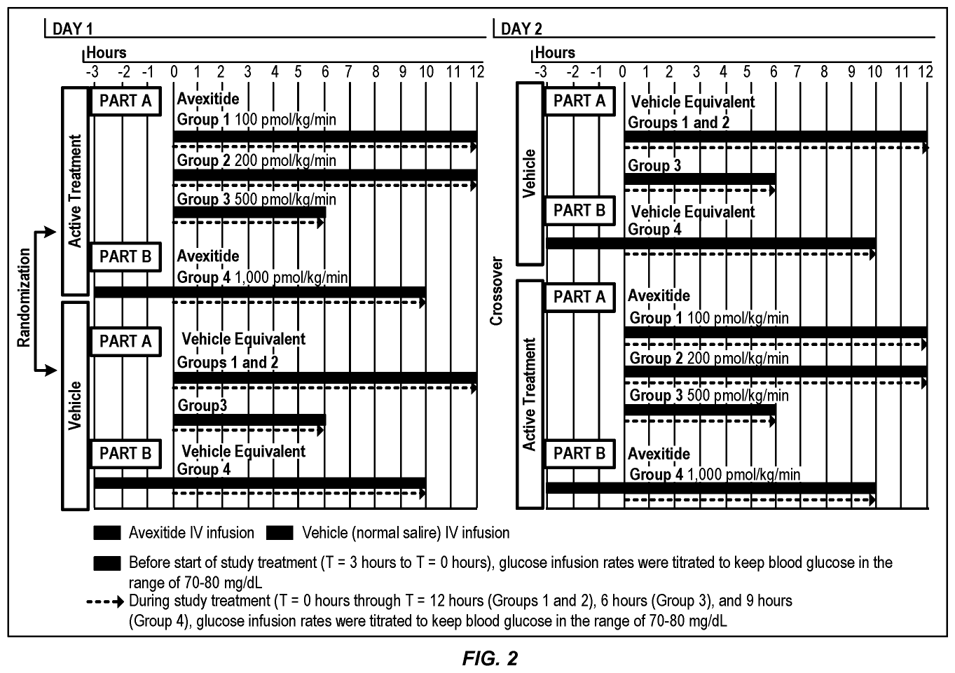

The study was divided into Parts A and B. See FIG. 2. With the exception of the first patient, all patients continued regular feedings every 3 hours throughout the study duration while receiving up to 12 hours of continuous intravenous infusion of avexitide or placebo on two separate days in crossover design. Study procedures were adjusted after the first patient to standardize the feeding interval. Patients in Part B but not Part A received an initial 3-hour infusion of glucose titrated to euglycemia and the dextrose infusion rate was adjusted to maintain glucose in the range of 70-90 mg/dL with repeat glucose levels every 15 minutes after each adjustment before the start of the study treatment. The study run-in period was timed to start with a feeding in the morning and was kept consistent between Part B study days.

As defined in the study protocol, the primary efficacy endpoint for Part A was the glucose infusion rate (GIR) at the end of the treatment period in Part A, and the primary efficacy endpoint as defined in the study protocol for Part B was the GR during the last 2 hours of the treatment period. Post-hoc analysis of PD data employed in the current PK/PD analysis defined GIR as the mean glucose infusion rate during the final 6 hours of avexitide or placebo infusion. GIR redefined for the current analyses as mean GIR during the final 6 hours of infusion (rather than the final 2 hours of infusion), as this is believed to be a more clinically meaningful endpoint, given that 2 hours is likely an insufficient duration over which to observe meaningful changes in GER. Additional study information is provided in Table 2. The avexitide infusion doses these subjects received is summarized in Table 3.

TABLE 2

| Intervention | Drug: Avexitide | |
|---|---|---|
| | A short-term intravenous infusion of the investigational drug, Avexitide, will | |
| | be administered over up to 9 hours. | |
| | Drug: Vehicle | |
| | A short-term intravenous infusion of normal saline (0.9% NaCl), or the | |
| | vehicle, will be administered over up to 9 hours. | |
| | Other Name: Placebo | |
| Study Arms | Each cohort will be administered the specified infusion rate of avexitide and vehicle | |
| | (normal saline), via continuous intravenous infusion, over 9 hours on two separate days | |
| | in random order, with 3 hours of follow-up after the last dose is administered or until | |
| | blood glucose is <70 mg/dL (whichever comes first). Glucose infusion rates (GIR) | |
| | will be titrated three hours prior to infusions to keep blood glucose in the range of 70- | |
| | 90 mg/dL. During both infusions, blood glucose will be measured every 30 minutes. | |
| | Experimental Group 1: Avexitide 0.02 mg/kg/hr | |
| | Experimental Group 2: Avexitide 0.04 mg/kg/hr | |
| | Experimental Group 3: Avexitide 0.10 mg/kg/hr | |
| | Experimental Group 4: Avexitide 0.20 mg/kg/hr | |
| | Interventions: | |
| | Drug: Avexitide | |
| | Drug: Vehicle | |
| Eligibility | Inclusion Criteria: | |
| Criteria | 1. | Confirmed clinical diagnosis of congenital hyperinsulinism |
| | 2. | Infants less than 12 months of age at study enrollment |
| | 3. | Failure to respond to treatment with diazoxide |
| | Exclusion Criteria: | |
| | 1. | Evidence of a medical condition that might alter results, including kidney |
| | | failure, severe liver dysfunction, severe respiratory or cardiac failure |
| | 2. | Treatment with medications that may affect glucose metabolism at the time of |
| | | initiation of study procedures, including: |

| | |
|---|---|
| a. | Treatment with glucagon 4 hours prior to infusion (T = 0) |
| b. | Treatment with octreotide 24 hours prior to infusion (T = 0) |
| c. | Treatment with diazoxide 72 hours prior to infusion (T = 0) |

| | |
|---|---|
| 3. | Suspected Beckwith-Wiedemann syndrome or other syndromic forms of |
| | congenital hyperinsulinism. |

| | |
|---|---|
| | All sexes eligible for study. |
| Primary | Mean Glucose Infusion Rate (GIR) [Time Frame: Up to 9 hours after the |
| Outcome | initiation of infusion] |
| Measures | To assess the effect of avexitide on glucose infusion rate, glucose infusion |
| | rate (GIR) over the last 2 hours of the treatment period was calculated by |
| | adding the total amount of intravenous glucose (mg) received over 2 hours |
| | divided by the weight (kg) and by time (120 min) during infusion of |
| | avexitide and normal saline vehicle. |
| | *As noted above, this outcome measure was re-defined post-hoc to GIR over |
| | the last 6 hours of the treatment period. |
| | To Determine the Pharmacokinetics of Avexitide [Time Frame: Up to 12 hours |
| | after the initiation of infusion] |
| | The following PK variables of interest include AUCO-∞, AUC0-t, maximal |
| | concentration (Cmax), time to maximal concentration (Tmax), concentration |
| | at end of infusion (Ceoi), steady state volume of distribution (Vss), clearance |
| | (CL) and half-life (t½) of Avexitide. These will be derived through both |
| | non-compartmental and model-based methods. |
| Secondary | Safety and Tolerability of Avexitide [Time Frame: Up to 24 hours post- |
| Outcome | infusion] |
| Measures | Number of participants with adverse events as a measure of safety and |
| | tolerability [evaluated by the result of laboratory safety tests (hematology, |
| | chemistry, urinalysis), vital signs, physical examinations, and 12-lead ECG] |
| | Mean Plasma Insulin [Time Frame: Up to 9 hours after the initiation of |
| | infusion] |
| | To assess the effect of Avexitide on plasma insulin levels, samples were |
| | collected at various time points during the infusion [avexitide or vehicle] |
| | including: at the start of the infusion (T = 0) and at 1, 5, and 9 hours post |
| | initiation of the infusion. |
| | Mean Plasma Glucose [Time Frame: Up to 9 hours after the initiation of |
| | infusion] |
| | To assess the effect of Avexitide on plasma glucose levels, samples were |
| | collected at various time points during the infusion [avexitide or vehicle] |
| | including: at the start of the infusion (T = 0) and at 1, 5, and 9 hours post |
| | initiation of the infusion. |
| | Mean Betahydroxybutyrate Levels [Time Frame: Up to 12 hours after the |
| | initiation of infusion] |
| | To assess the effect of Avexitide on mean betahydroxybutyrate levels, |
| | samples were collected at various time points during the infusion [avexitide |
| | or vehicle] including: at the start of the infusion (T = 0) and hourly up to 12- |
| | hours post initiation of the infusion. |

TABLE 3

| Dose Scheme | | | Total | Number of |
|---|---|---|---|---|
| Avexitide Infusion Rate | | Number of | Avexitide | Completed |
| pmol/kg/min | mg/kg/hour | Hours | Dose (mg/kg) | Patients |

Part A

| | | | | | |
|---|---|---|---|---|---|
| 1 | 100 | 0.02 | 12 | 0.24 | 5 |
| 2 | 200 | 0.04 | 12 | 0.48 | 2 |
| 3 | 500 | 0.1 | 6 | 0.6 | 2 |

Part B

| | | | | | |
|---|---|---|---|---|---|
| 4 | 1000 | 0.2 | 9 | 1.8 | 4 |

Patient Baseline Characteristics. This study enrolled infants with congenital hyperinsulinism owing to KATP channel mutations who are unresponsive to medical therapy and will require a pancreatectomy to control hypoglycemia at the Children's Hospital of Philadelphia. Participants were recruited based on a confirmed clinical diagnosis of congenital hyperinsulinism at 1 academic medical center between August 2009 and October 2019. The first participant was enrolled on Aug. 26, 2008 and the last participant was enrolled in Jan. 25, 2017. Of the 14 enrolled participants, 13 met inclusion criteria and were randomized to treatment. Patient demographics and baseline characteristics are shown in Table 4 and Table 5 below.

Study participants included 13 neonates and infants age 11 days to 5 months; 7F/6M. Nearly all patients either failed or were not candidates for diazoxide, octreotide, or glucagon treatment, Diazoxide was trialed and failed by all subjects with the exception of subject 6, who did not undergo a diazoxide trial because the patient's confirmed genetic mutation implied diazoxide resistance, Octreotide was trialed and failed by all subjects through subject 4. For subsequent subjects (with the exception of subject 12) octreotide was not trialed because standard of care changed such that octreotide was no longer used. For subject 12, octreotide was trialed followed by washout, avexitide/placebo infusion, then partial pancreatectomy, with central line retained intact throughout. Glucagon was not used as part of standard of care with the exception of subjects 1 and 6, for whom IV glucagon was trialed due to concerns for fluid overload followed by washout, participation in Study 2008-10-6256, then partial pancreatectomy (subject 1) or subtotal pancreatectomy (subject 6). In both instances, the central line remained intact during and after glucagon treatment until after pancreatectomy.

TABLE 4

| Subject | Age | Sex | Weight | | | | | |
|---|---|---|---|---|---|---|---|---|
| ID | (wk) | (M/F) | (kg) | Histology | Gene | Diazoxide | Octreotide* | Glucagon |
| 1 | 4 | F | 3.92 | Focal | KCNJ11 | Failed | Failed | Trialed |
| 2 | 6 | F | 4.88 | Focal | ABCC8 | Failed | Failed | Not |
| | | | | | | | | trialed |
| 3 | 9 | M | 6.4 | Diffuse | ABCC8 | Failed | Failed | Not |
| | | | | | | | | trialed |
| 4 | 21 | F | 6.47 | Focal | ABCC8 | Failed | Failed | Not |
| | | | | | | | | trialed |
| 5 | 3 | F | 6.245 | Diffuse | ABCC8 | Failed | Not | Not |
| | | | | | | | trialed | trialed |
| 6 | 2 | F | 4.1 | Diffuse | ABCC8 | Not | Not | Trialed |
| | | | | | | trialed** | trialed | |
| 7 | 6 | M | 4.41 | Focal | ABCC8 | Failed | Not | Not |
| | | | | | | | trialed | trialed |
| 8 | 21 | F | 6.9 | Focal | ABCC8 | Failed | Not | Not |
| | | | | | | | trialed | trialed |
| 9 | 13 | M | 6.6 | Focal | ABCC8 | Failed | Not | Not |
| | | | | | | | trialed | trialed |
| 10 | 6 | M | 5.3 | Focal | KCNJ11 | Failed | Not | Not |
| | | | | | | | trialed | trialed |
| 11 | 5 | F | 4.79 | Focal | ABCC8 | Failed | Not | Not |
| | | | | | | | trialed | trialed |
| 12 | 35 | M | 6.6 | Focal | ABCC8 | Failed | Trialed*** | Not |
| | | | | | | | | trialed |
| 14 | 35 | M | 8.2 | Focal | ABCC8 | Failed | Not | Not |
| | | | | | | | trialed | trialed |

*During the course of this study, clinical practice changed such that octreotide was no longer standard of care.
**Diazoxide not trialed because genetic mutation implied diazoxide resistance.
***Octreotide trialed with partial efficacy but was discontinued for PET imaging followed by focal excision.

TABLE 5

| | Treatment Arms | | | | |
|---|---|---|---|---|---|
| | Expt | Expt | Expt | Expt | |
| | Group 1 | Group 2 | Group 3 | Group 4 | Total |
| Overall # of | 5 | 2 | 2 | 4 | 13 |
| baseline | | | | | |
| participants | | | | | |
| Sex: | | | | | |
| Female | 4 | 1 | 1 | 1 | 7 |
| Male | 1 | 1 | 1 | 3 | 6 |
| Ethnicity | | | | | |
| (NIH/OMB): | | | | | |
| Hispanic or | 1 | | | 2 | 3 |
| Latino | | | | | |
| Ethnicity | | | | | |
| (NIH/OMB): | | | | | |
| Not Hispanic | 4 | 2 | 2 | | 10 |
| or Latino | | | | | |

Data Handling. Patients were defined as evaluable for population pharmacokinetic (PopPK) analysis if they had at least one adequately documented avexitide administration and a corresponding PK sample collection after the dose. If the time of drug administration or sample collection were missing, the records were excluded from analysis. Assembly of the PopPK dataset was performed R (version 4.0.2). The NONMEM data file was created as a Microsoft Excel format (.csv) for input into NONMEM.

Population PK and PKIPD Analysis Method. The PopPK analysis methods were based on the Food and Drug Administration (FDA) Guidance for Industry Population Pharmacokinetics and Committee for Medicinal Products for Human Use (CHMP) Guideline on Reporting the Results of Population Pharmacokinetic Analysis. The PopPK analysis was performed using the non-linear mixed effects modeling approach. Model parameter estimation were implemented with NONMEM 7, version 7.4 (ICON Development Solutions. Ellicott City, Maryland, USA) and R (Version 4.0.2) based on Beal, S. L., et al., NONMEM User's Guide, Part I-VII. San Francisco: UCSF: University of California at San Francisco; 1988-1992. PopPK estimation was performed using the first-order conditional estimation (FOCE) method in NONMEM.

Avexitide PopPK in Neonate and Infant Patients with CHI. An external validation process was implemented to estimate avexitide PK in neonate patients with CHI. A previously established avexitide PopPK model was utilized in the external validation. See Ng C. et al, Population pharmacokinetics of exendin-(9-39) and clinical dose selection inpatients with congenital hyperinsulinism, Br. J. Clin. Pharmacol. 2018 March; 84(3):520-532.

Predicted avexitide plasma concentrations for validation patients were obtained by fixing the parameters in the structural and variance model to the parameter estimates in the final model using post-hoc Bayesian forecasting with NONMEM 7, with the $ESTIMATION command set as MAXEVAL=0.

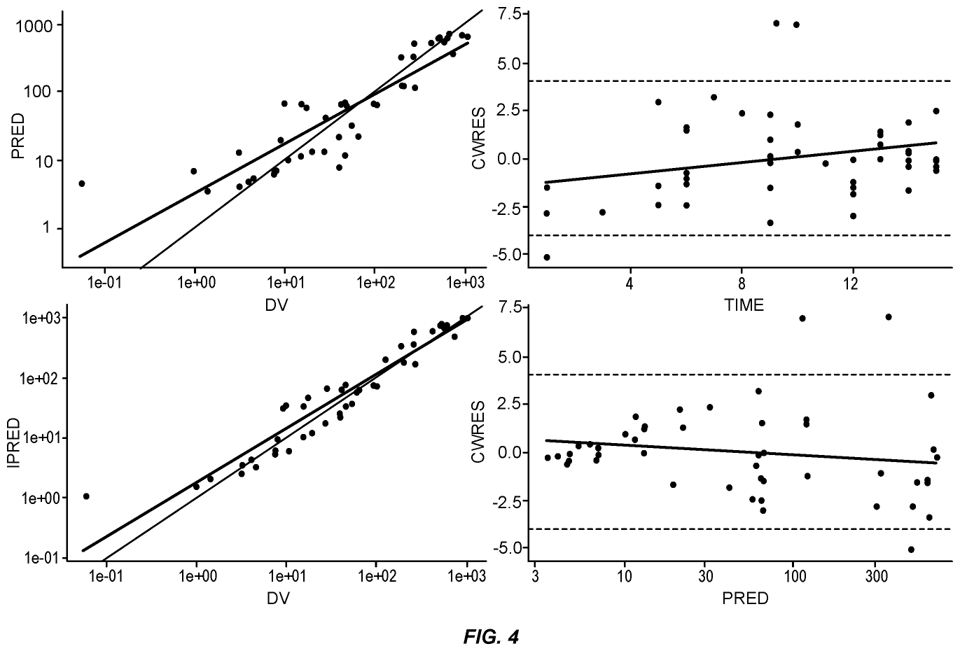

Model diagnostic graphs were generated as follows: population predicted concentrations (PRED) versus observed concentrations (DV), the individual predicted concentrations (IPRED) versus DV, and the individual weighted residuals (IWRES) or CWRES versus PRED or time. See FIG. 4. This set of diagnostic graphs showed whether the predicted concentrations matched the observed concentrations.

PK PD Analysis for CHI. The relationship between avexitide PK vs. PD endpoints (change in mean GIR as measured over the final 6-hours of avexitide infusion vs. vehicle infusion) were explored graphically initially, followed by evaluation of potential PK/PD models (linear or Emax). PopPK model predicted average individual avexitide plasma concentrations for the duration of GIR measurements were used to correlate the absolute change in GIR vs baseline GIR before the initiation of the avexitide infusion.

Simulations of Avexitide PK Following SC Administration. Avexitide plasma concentrations in neonate and infant patients following SC administration was simulated based on a previously established PopPK model for avexitide following SC administration in adult subjects. Allometric scaling was implemented to derived neonate and infant PK parameters using scaling factor of 0.75 for clearance and 1 for volume. Typical avexitide plasma concentrations were simulated assuming BID or TID treatment at various weight-based dose levels.

### B. Results

PopPK Modeliig of Avexitide in Neonate and Infant CHI Patients. A total of thirteen neonates and infants (age, 11 days to 5 months) with KATP CHI were enrolled into this study. Among them, 9 subjects were enrolled into Part A and received avexitide infusions at a rate of 0.02 (N=6), 0.04 (N=2), and 0.1 (N=2) mg/kg/h, respectively. In Part B, 4 subjects received avexitide infusions at a rate of 0.2 mg/kg/h.

Eleven out of 13 neonate and infant patients had at least one plasma sample with avexitide measurement. As such, the external validation included these 11 subjects.

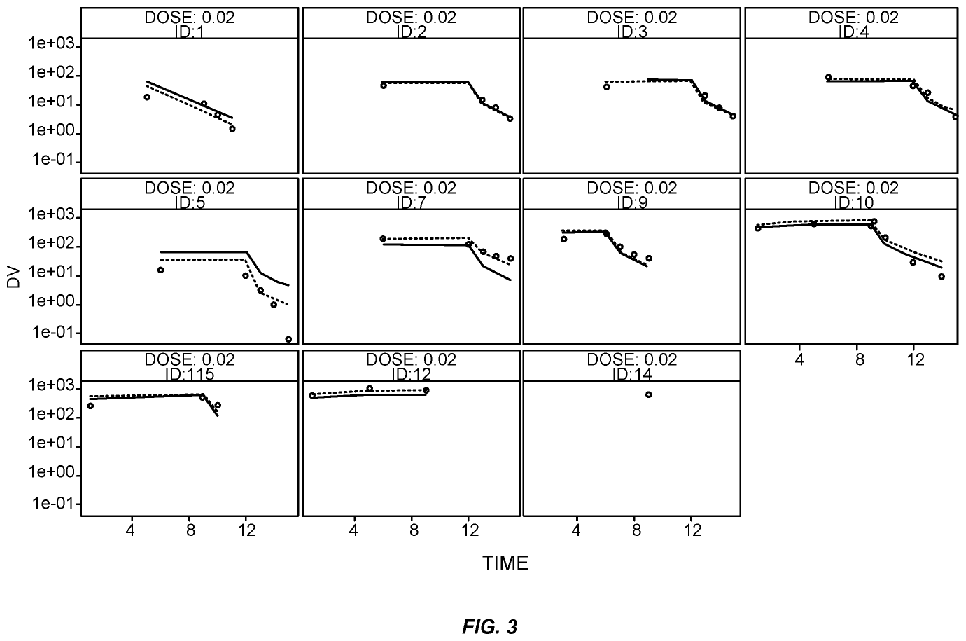

Individual fitting of the concentration-time profiles from the external validation are presented in FIG. 3. In FIG. 3, circles indicate observed avexitide plasma concentrations, black lines indicate population prediction of avexitide concentration-time profiles, and red lines indicate individual prediction of avexitide concentration-time profiles.

The general goodness-of-fit plots of the external validation are shown in FIG. 4. In particular, FIG. 4 shows: (a) individual predicted (IPRED) plasma avexitide concentrations versus observed avexitide concentrations on a logarithmic scale (bottom left); (b) population predicted (PRED) plasma avexitide concentrations versus observed plasma avexitide concentrations on a logarithmic scale (top left); (c) conditional weighted residuals (CWRES) of plasma avexitide concentrations versus time (top right); and (d) CWRES versus PRED (bottom right). In these, points are individual data, solid black lines represent the unit diagonal, and blue solid lines represent the unit line at zero. A good agreement between the predicted concentrations and the observed concentrations was observed.

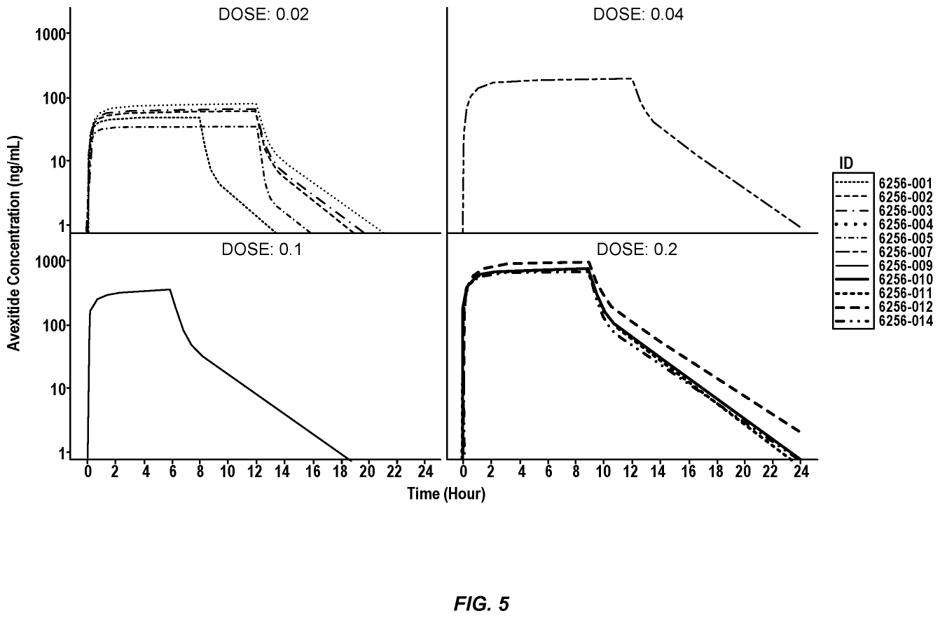

FIG. 5 shows the model predicted individual avexitide concentration-time profiles following avexitide IV infusion.

Glycemic responses to Avexitide vs. Placebo infusion. Mean GIR results following avexitide or vehicle infusion, as defined as the mean GIR during the final 6 hours of avexitide or vehicle infusion, for each patient is presented in Table 6. Eleven out of 13 neonate and infant patients had at least 8 hours of avexitide and placebo infusion, allowing for 2 hours to achieve steady state concentrations followed by another 6 hours of available GIR data. Additionally, 10 out of the 11 patients with sufficient GIR data also had PK data. Therefore, a total of 10 patients were included in the PK/PD analysis.

TABLE 6

| | | | | Absolute | Percent |
|---|---|---|---|---|---|
| | AVX infusion rate | GIR (mg/kg/min) | | Difference | Difference |
| ID | (pmol/kg/min) | PBO | AVX | in GIR | in GIR |
| 1 | 100 | 10.9 | 7.4 | 3.5 | 32% |
| 2 | 100 | 1.2 | 3.6 | −2.4 | −196% |
| 3 | 100 | 6.5 | 4.8 | 1.7 | 26% |
| 4 | 100 | 5.4 | 2.5 | 2.9 | 54% |
| 5 | 100 | 18.9 | 17.9 | 0.9 | 5% |
| 6 | 200 | 12.8 | 12.1 | 0.7 | 6% |
| 7 | 200 | 11.5 | 6.4 | 5.1 | 44% |
| 8 | 400 | | | N/A^{a} | |
| 9 | 400 | | | N/A^{a} | |
| 10 | 1000 | 5.4 | 3.8 | 1.6 | 29% |
| 11 | 1000 | 8.4 | 0 | 8.4 | 100% |
| 12 | 1000 | 12.6 | 9.2 | 3.4 | 27% |
| 13 | N/A^{b} | | | N/A^{b} | |
| 14 | 1000 | 4.2 | 0.3 | 3.9 | 93% |

^{a}Avexitide (AVX) infusion duration was insufficient to determine GIR at steady state concentrations.
^{b}Subject was withdrawn prior to avexitide dosing.

Table 7 shows mean GIR results by dose level. Relative to placebo, avexitide treatment significantly reduced GIR when evaluated across all dose levels (p=0.0087), with reductions in GIR observed in 10 participants, and dose-dependent improvements observed. On average, relative to placebo, avexitide infused at 100, 200 and 1000 pmol/kg/min demonstrated 1.3 (15%), 2.9 (24%), and 4.3 (56%) mg/kg/min reductions in GIR. Additionally, avexitide treatment reduced the GIR from above 10 mg/kg/min during vehicle to below this threshold in 3 participants (subjects 1, 7 and 12), and entirely abolished the glucose infusion requirement in 2 participants (subjects 11 and 14) who received 1000 pmol/kg/min.

TABLE 7

| Avexitide | | Mean GIR during | Mean GIR during | Mean Absolute | Mean | |
|---|---|---|---|---|---|---|
| infusion rate | | placebo infusion | avexitide infusion | Difference in GIR | Percent | |
| (pmol/kg/min) | | (mg/kg/min) | (mg/kg/min) | (mg/kg/min) | Difference | P-value |
| 100 | (n = 5) | 8.6 | 7.2 | 1.3 | 15% | 0.323 |
| 200 | (n = 2) | 12.2 | 9.3 | 2.9 | 24% | 0.438 |
| 1000 | (n = 4) | 7.6 | 3.3 | 4.3 | 56% | 0.058 |
| All | (n = 11) | 8.9 | 6.2 | 2.7 | 30% | 0.009 |

As shown in Table 7, avexitide treatment significantly reduced GIR across all dose levels (p=0.009), demonstrated dose-dependent improvements in GIR (including 56% reduction in GIR at top dose evaluated), and entirely abolished glucose infusion requirements in (50%) of participants at top dose. Furthermore, the avexitide treatment was well-tolerated, with no treatment-related adverse events or severe adverse events.

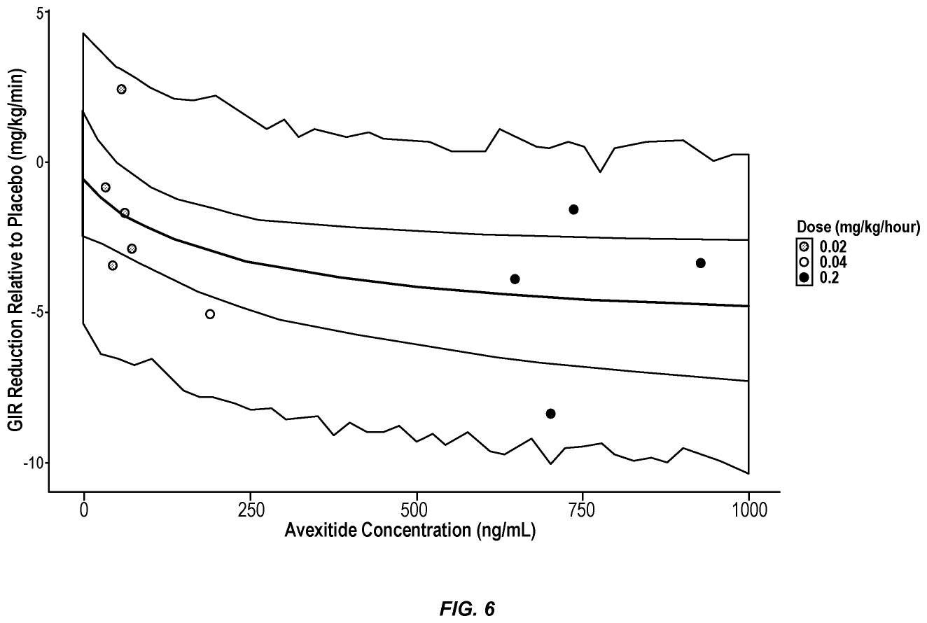

PK/PD Analysis. Absolute change in mean GIR during the final 6 hours of avexitide infusion relative to vehicle infusion was evaluated against the PopPK model-predicted average avexitide concentration for the duration of the GIR measurement. The PK/PD data were fitted to an Emax model:

$$E = {E}_{0} + \frac{{E}_{max} \times C}{{EC}_{50} + C} ,$$

where C represents avexitide concentration, E₀ represents baseline response, Eₘₐₓ represents maximal response, and EC₅₀ represents exposure that achieves 50% of maximal response. Model fitting results are shown in FIG. 6. In FIG. 6, the black line indicates a model predicted mean response, the dark grey area indicates 90% CI, the light grey area indicates 90% PI, and the circles indicate observed absolute change in GIR (ng/mL).

The parameters predicted by the Eₘₐₓ model are presented in Table 8. The outcome of Eₘₐₓ modeling estimated the plasma avexitide EC50 to be 291 ng/mL.

TABLE 8

| Parameter | Estimates | SE |
|---|---|---|
| Emax (mg/kg/min) | −5.6 | 0.12 |
| EC50 (ng/mL) | 291 | 7.55 |
| E0 (mg/kg/min) | −0.53 | 0.05 |

PK Simulation Results. Typical avexitide PK profiles in neonate and infant patients receiving avexitide as a subcutaneous administration at different BID and TID dosing regimens were simulated based on PK parameters from a previously established subcutaneous avexitide PopPK model on adult subjects with body weight based allometric scaling.

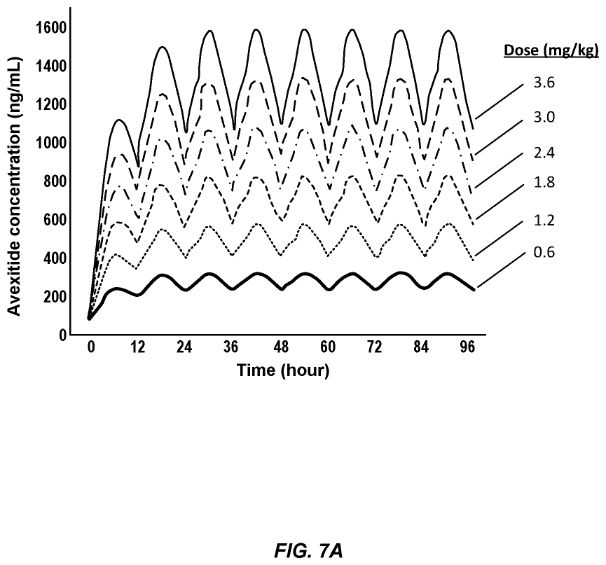

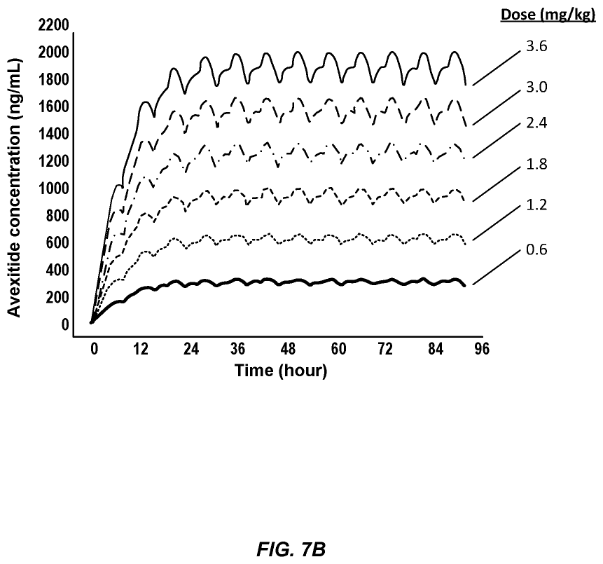

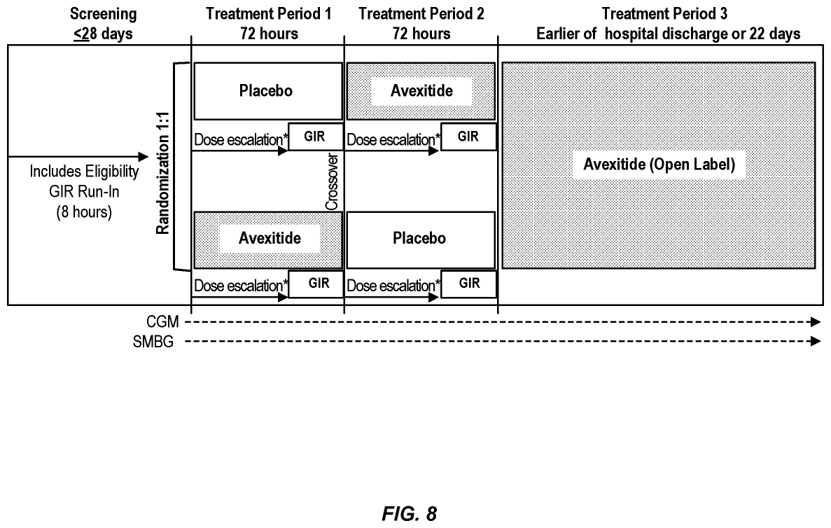

Six different treatment regimens were included in the simulations: 0.6, 1, 2, 1.8, 2.4, 3, and 3.6 mg/kg BID and TID. The simulated PK profiles following these dosing regimens are presented in FIG. 7A-7B. These PK simulations indicated that avexitide administered at or above 0.6 mg/kg TID or 1.2 mg/kg BID will achieve steady-state plasma concentration above the EC50 of 291 ng/mL. In addition, BID or TID subcutaneous dosing of avexitide will allow plasma avexitide concentrations to reach steady-state after approximately 2 days of dosing.

### C. Conclusions

Avexitide administered as a continuous infusion at rates ranging from 100-1000 pmol/kg/min demonstrated significant reductions in glucose requirements to maintain euglycemia and demonstrated dose-dependent improvements in the treatment effect. An exposure-response relationship was observed. Furthermore, PK simulation results indicate that avexitide can effectively be administered as a SC injection dosed BID or TID in neonate and infant patients with CHI.

These results establish a significant effect of avexitide on reduction in GIR and therefore on the ability to discontinue the need for a central line in neonates and infants with CHI. Moreover, these results also provide preliminary clinical evidence of the ability to reduce the GIR to the extent to allow for potential discharge home, avoidance of the need for pancreatectomy, reduction in the extent of pancreatic tissue removed, and reduction in the risk for fluid overload. On the basis of these results, there is sufficient preliminary clinical evidence to support the avexitide treatment effect on the clinically significant endpoint.

### Example 2: Effect of Avexitide on Glucose Requirements to Maintain Euglycemia with a Confirmed Diagnosis of CHI (EIG-AVX-005-CHI)

### A. Methods

This will be a placebo-controlled, randomized, crossover study to evaluate the effect of avexitide on glucose requirements to maintain euglycemia in infants with a confirmed diagnosis of CHI. The primary aim of this study is to evaluate the effect of avexitide on glucose requirements to maintain euglycemia in hospitalized pediatric patients with congenital hyperinsulinism (CHI) based on genetic, clinical, and diagnostic workup consistent with the standard of care for patients with CHI, inclusive of Hyperinsulinemia (plasma insulin above the limit of detection of the assay documented during an event of hypoglycemia), Hypofattyacidemia (plasma free fatty acid <1.7 mmol/L), Hypoketonemia (Beta-hydroxybutyrate <1.8 mmol/L), and/or Glycemic response (an increase in plasma glucose of >30 mg/dL (>1.7 mmol/L) after IV or intramuscular glucagon administration). The secondary aims are to evaluate the effect of avexitide on discharge readiness among patients with diffuse disease with basal glucose infusion rate (GIR) >10 mg/kg/min as well as on hypoglycemia event rate (Levels 1 and 2) (European Association for the Study of Diabetes [EASD] and American Diabetes Association [ADA]) as measured by self-monitoring of blood glucose (SMBG) during Treatment Periods 1 and 2.

Where not otherwise specified herein, avexitide acetate will be used. The tested concentrations will be 40 mg/mL, 100 mg/mL, and 200 mg/mL. The placebo is an acetate buffered solution.

Study Design. A Phase 3, multicenter, double-blind, placebo-controlled, dose escalation, crossover study followed by an open-label period will be implemented. Patients will be randomized to 2 treatment sequences (avexitide-placebo or placebo-avexitide) in a 1:1 ratio. Approximately 14 patients will receive either 3 times daily (TID) subcutaneous (SC) injections of placebo for 72 hours followed by TID injections of avexitide for 72 hours, or TID SC injections of avexitide for 72 hours followed by TID injections of placebo for 72 hours, in crossover design.

All patients will receive SC injections of avexitide TID for 72 hours and SC injections of placebo TID for 72 hours in crossover design and random order. For patients ≥14 days of age, when initiating each 72-hour dosing regimen, a priming dose of 1.8-3.6 mg/kg will be administered, followed by a maintenance dose of 0.9-1.8 mg/kg TID (e.g., 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg) 8 hours later. For patients <14 days of age, when initiating each 72-hour dosing regimen, a priming dose of 0.9-1.8 mg/kg will be administered, followed by a maintenance dose of 0.9-1.8 mg/kg TID 8 hours later. The study drug dose will be escalated up to 3.6 mg/kg TID to efficacy as long as no safety or tolerability concerns have been observed (if the mean GIR #0) after mean GIR determination in the final 8 hours of each 24-hour period. The volume of placebo to be injected TID will match the volume of avexitide that would be administered.

Following the double-blind crossover phase (Treatment Periods 1 and 2), patients will participate in an open-label period (Treatment Period 3), during which all patients will continue to receive the dose to which they were optimized during the crossover period on an open-label basis. Treatment Period 3 will conclude at the time of discharge from the hospital or after 28 days of inpatient care, whichever is earlier. Upon study completion, all eligible patients can enroll in the subsequent Open-Label Extension (OLE) study described in Example 4 (referred to below as EIG-AVX-007-CHI).

As defined in the study protocol, one objective is to evaluate the effect of avexitide on the discharge readiness among patients with diffuse disease with basal GIR>10 mg/kg/min. A Run-In Period GIR will be established over an 8-hour period. Dextrose infusion rate will be adjusted to maintain euglycemia (see Table 9). CGM assessments will also be completed during the GIR Run-In Period. Additional study information is provided in Table 10.

TABLE 9
Exemplary Clinical Algorithm for Blood Glucose Adjustment

| Blood Glucose (mg/dL) | Glucose Infusion Rate (GIR) Adjustment |
|---|---|
| <50 | Give 200 mg/kg dextrose bolus and |
| | increase GIR by 2 mg/kg/min |
| 50-59 | Increase GIR by 2 mg/kg/min |
| 60-69 | Increase GIR by 1 mg/kg/min |
| 70-90 | Maintain current GIR |
| 91-100 | Reduce GIR by 1 mg/kg/min |
| 101-120 | Reduce GIR by 1.5 mg/kg/min |
| >120 | Reduce GIR by 2 mg/kg/min |

TABLE 10

| Intervention | SC injections of avexitide TID for 72 hours and SC injections of placebo TID for | |
|---|---|---|
| | 72 hours in crossover design and random order. | |
| | For patients ≥14 days of age, when initiating each 72-hour dosing regimen, a | |
| | priming dose of 1.8-3.6 mg/kg will be administered, followed by a maintenance | |
| | dose of 0.9-1.8 mg/kg TID 8 hours later and dose escalated to efficacy (mean | |
| | GIR ≠ 0) and tolerability to a maximum dose of 3.6 mg/kg TID.. For patients <14 | |
| | days of age, when initiating each 72-hour dosing regimen, a priming dose of 0.9- | |
| | 1.8 mg/kg will be administered, followed by a maintenance dose of 0.9-1.8 mg/kg | |
| | TID 8 hours later, and dose escalated thereafter to efficacy (mean GIR ≠ 0) and | |
| | tolerability to a maximum dose of 3.6 mg/kg TID. | |
| | During the up-to-22-day open label period, patients will receive the dose to which | |
| | they were optimized during the crossover period every 8 hours and blood glucose | |
| | will continue to be sampled by Nova meter. | |
| Study Arms | Patients will be randomized to 2 treatment sequences (avexitide-placebo or | |
| | placebo-avexitide) in a 1:1 ratio. | |
| Eligibility | Inclusion Criteria: | |
| Criteria | 1. | Consenting guardian is able to understand the purpose and risks of the study; |
| | | adhere to scheduled visits, treatment plans, laboratory tests, procedures, and |
| | | study evaluations; and provide written informed consent; |
| | 2. | Male or female, 0 to 364 days old (inclusive) at Screening; |
| | 3. | Confirmed diagnosis of CHI, based on genetic, clinical, and diagnostic |
| | | workup consistent with the standard of care for patients with CHI, inclusive |
| | | of the following criteria during hypoglycemia: |

| | |
|---|---|
| a. | Hyperinsulinemia: plasma insulin above the limit of detection of the |
| | assay documented during an event of hypoglycemia; |
| b. | Hypofattyacidemia: plasma free fatty acid <1.7 mmol/L; |
| c. | Hypoketonemia: Beta-hydroxybutyrate <1.8 mmol/L; and/or |
| d. | Glycemic response: an increase in plasma glucose of >30 mg/dl (>1.7 |
| | mmol/L) after IV or intramuscular glucagon administration. |

| 4. | Requires continuous IV glucose to prevent hypoglycemia (glucose <70 |
|---|---|
| | mg/dL) as confirmed during an 8-hour Screening GIR assessment Run-In. |
| Exclusion Criteria: | |
| 1. | Receipt of an investigational or other study drug within 5 half-lives of the |
| | drug, not inclusive of receipt of ¹⁸F-DOPA (¹⁸F-dihydroxyphenylalanine), |
| | used during positron emission tomography (PET) imaging; |
| 2. | Participation in another interventional clinical study within 30 days before |
| | Screening; |
| | Note: Participation in studies employing investigational use of PET imaging |
| | within 30 days is acceptable. |
| 3. | Known or suspected allergy to the study drug or related products; |
| 4. | Clinically significant active infection within 14 days before the first dose of |
| | study drug; |
| 5. | Suspected of having a transient form of CHI (e.g., transient hyperinsulinism |
| | due to maternal diabetes, perinatal stress, or intrauterine growth retardation); |
| 6. | Evidence of other potential cause of hypoglycemia (not CHI); |
| 7. | Any clinically significant abnormality that, in the opinion of the Investigator, |
| | would affect the patient's ability to participate in the study; |
| 8. | Presence of any clinically relevant renal, hepatic, pancreatic, cardiovascular, |
| | neurological, hematological, pulmonary, or gastrointestinal abnormality that |
| | may preclude the individual from safely completing the study; |
| | Note: Patients with pancreatic abnormalities related to CHI and/or treatment |
| | thereof (eg, history of pancreatomy) are eligible. |
| 9. | Presence of any malignancy; |
| 10. | Abnormal liver function, defined as aminotransferase (alanine |
| | aminotransferase or aspartate aminotransferase) levels >2.5 × upper limit of |
| | normal (ULN) reference range, and/or total bilirubin level >2 × ULN at |
| | Screening; |
| | Note: Neonates with total bilirubin level <4 × ULN at Screening with a |
| | clinical diagnosis of physiological jaundice (within first 14 days of life) or |
| | breast-feeding jaundice are eligible if, per Investigator judgement, |
| | hyperbilirubinemia is resolving. |
| 11. | Abnormal renal function for the patient age, as calculated using the Schwartz |
| | formula at Screening; |
| 12. | Physical examination or electrocardiogram (ECG) with clinically significant |
| | abnormality, as judged by the Investigator, at Screening; |
| 13. | Current use of medication(s) that affect(s) glucose metabolism, such as |
| | systemic corticosteroids, e.g., hydrocortisone >20 mg/m² body surface area |
| | or equivalent, glucagon, or octreotide. Note: Effective or partially effective |
| | concomitant medications used for treatment of hypoglycemia will not be |
| | withdrawn and patients will not be withdrawn from concomitant therapies in |
| | order to be eligible for study participation. If, in the opinion of the |
| | Investigator, the patient is experiencing a clinical benefit from diazoxide, |
| | continued use will be permitted, provided the dose remains stable throughout |
| | the Run-In Period and each of Treatment Periods 1 and 2; or |
| 14. | Prior use of lanreotide, sirolimus (mechanistic target of rapamycin |
| | inhibitors), anti-inflammatory biological agents, or other immune modulating |
| | agents. |

| | |
|---|---|
| Primary | GIR required to maintain euglycemia, defined as the percent change from |
| Outcome | the mean Run-In GIR (from the Run-In Period) to the mean GIR during the |
| Measures | final 24-hour infusion of each treatment period (Treatment Periods 1 and 2). |
| Secondary | Proportion of patients who achieve mean GIR <10 mg/kg/min for at least 24 |
| Outcome | hours; |
| Measures | Time to mean GIR <10 mg/kg/min for at least 24 hours; |
| | Hypoglycemia event rate (Level 1), defined as the number of events with |
| | glucose <70 mg/dL or <3.9 mmol/L, as measured by SMBG during each of |
| | Treatment Periods 1 and 2; |
| | Clinically important hypoglycemia event rate (Level 2), defined as the |
| | number of events with glucose <54 mg/dL or <3.0 mmol/L, as measured by |
| | SMBG during each of Treatment Periods 1 and 2; |
| | TIR, defined as the change in the percentage of time, compared to the |
| | percentage of time from the Run-In Period, in the glucose range of 70 to 180 |
| | mg/dL (3.9 to 10.0 mmol/L), inclusive, as measured by CGM during each of |
| | Treatment Periods 1 and 2; and |
| | TBR, defined as the change in the percentage of time, compared to the |
| | percentage of time from the Run-In Period, in hypoglycemia (Level 1 and |
| | Level 2 hypoglycemia; glucose <70 mg/dL or <3.9 mmol/L and glucose <54 |
| | mg/dL or <3.0 mmol/L), as measured by CGM during each of Treatment |
| | Periods 1 and 2; |
| | Time to IV dextrose discontinuation, defined as the time from start of |
| | Treatment Period 3 until the patient has completed weaning from IV dextrose |
| | infusion indicating the patient does not require inpatient services to maintain |
| | euglycemia; |
| | Time to central line removal; |
| | Proportion of patients who did not require pancreatectomy; |
| | Hypoglycemia event rate (Level 1), defined as the number of events with |
| | glucose <70 mg/dL (<3.9 mmol/L), as measured by SMBG during Treatment |
| | Period 3; |
| | Clinically important hypoglycemia event rate (Level 2), defined as the |
| | number of events with glucose <54 mg/dL (<3.0 mmol/L), as measured by |
| | SMBG during Treatment Period 3; |
| | TIR, defined as the percent time in glucose range of 70 to 180 mg/dL (3.9 to |
| | 10.0 mmol/L), inclusive, as measured by CGM during Treatment Period 3; |
| | TBR, defined as the percent time in hypoglycemia (glucose <70 mg/dL or |
| | <3.9 mmol/L and glucose <54 mg/dL or <3.0 mmol/L), as measured by CGM |
| | during each of Treatment Period 3; |
| | Total carbohydrate requirements, defined as the amount (g) of carbohydrates |
| | administered via IV glucose infusion, as part of total parenteral nutrition, and |
| | via nasogastric tube or gastrostomy tube during Treatment Period 3; and |
| | Development of antidrug antibody (ADAb). |
| | Evaluation of the PK profile of avexitide. |

### Example 3: Efficacy, Safety, and Pharmacokinetics of Avexitide in Children with CHI (EIG-AVX-006-CHI)

This will be a placebo-controlled study to assess the efficacy, safety, and pharmacokinetics of avexitide in children up to 18 years old with CHI. The primary aim of this study is to evaluate the effect of the addition of avexitide to standard of care on the hypoglycemia event rate (Level 1) (EASD and ADA) as measured by self-monitoring of blood glucose (SMBG). The secondary aims are to determine the hypoglycemia event rate (Level 1) as measured by continuous glucose monitor (CGM) (EASD and ADA), the clinically important hypoglycemia event rate (Level 2) as measured by CGM and SMBG (EASD and ADA), the time in range (TIR) and time below range (TBR) in Levels 1 and 2 hypoglycemia as measured by CGM, and the total carbohydrates administered to treat or prevent hypoglycemia.

Where not otherwise specified herein, avexitide acetate is used. The drug product compositions consist of 40 mg/mL, 100 mg/mL, and 200 mg/mL avexitide. The placebo is an acetate buffered solution.

Study Design. A Phase 3, multicenter, randomized, double-blind, placebo-controlled study of avexitide in children with CHI will be implemented. Eligible patients must have a confirmed diagnosis of CHI and an average of at least 3 documented episodes of hypoglycemia (<70 mg/dL [<3.9 mmol/L]) per week (at least 6 hypoglycemia episodes during the 2-week Run-In Period). Approximately 30 patients will be randomized and assigned in a 1:1 ratio to the following treatment arms, each treatment comprising 10 weeks of dosing by SC injection. Patients will undergo Screening within 8 weeks of randomization and enrollment, during which eligibility will be confirmed based on the frequency of hypoglycemia events during the Run-In Period. The Run-In Period is a 2-week period and is considered part of the 8-week Screening Period. The study will consist of a 10-week Treatment Period. Upon study completion, all eligible patients can enroll in the subsequent Open-Label Extension (OLE) study described in Example 4 (referred to below as EIG-AVX-007-CHI). Additional study information is provided in Table 11.

Avexitide doses ranging from 0.9 to 3.6 mg/kg administered BID will be evaluated. Avexitide (or matching placebo) will be injected in a single dose volume as a function of body weight range (Table 12). For patients with body weight exceeding 56 kg, doses are to be administered following a fixed-dose regimen escalating from 50 mg BID to 100 mg BID, to 150 mg BID, to 200 mg BID (maximum dose). If the patient experiences ≥1 hypoglycemia (Level 1) event per week, as recorded in the patient eDiary, the dose will be escalated stepwise (0.9 mg/kg, then 1.8 mg/kg, then 2.7 mg/kg, and then 3.6 mg/kg), as tolerated. If a dose level is not well tolerated, the dose level will be decreased stepwise.

TABLE 11

| Intervention | 10 weeks of dosing by subcutaneous (SC) injection; Placebo SC, twice daily | |
|---|---|---|
| | (BID) every 12 hours; or Avexitide SC, BID every 12 hours | |
| Study Arms | Approximately 30 patients are planned to be randomly assigned into 2 arms | |
| | with 1:1 ratio (15 patients in avexitide and 15 patients in placebo). | |
| Eligibility | Inclusion Criteria: | |
| Criteria | 1. | Consenting caregiver/guardian is able to understand the purpose and risks of |
| | | the study; adhere to scheduled visits, treatment plans, laboratory tests, |
| | | procedures, and study evaluations; and provide written informed consent. |
| | | Assent of participant will be obtained per institutional standards; |
| | 2. | Male or female, up to 18 years of age at Screening Visit: |
| | 3. | Previously documented diagnosis of CHI, consistent with the standard of care |
| | | for patients with CHI, meeting 1 or more of the following criteria during |
| | | hypoglycemia: |

| | |
|---|---|
| a. | Hyperinsulinemia: plasma insulin above the limit of detection of the |
| | assay documented during an event of hypoglycemia; and/or |
| b. | Hypofattyacidemia: plasma free fatty acid <1.7 mmol/L; and/or |
| c. | Hypoketonemia: Beta-hydroxybutyrate <1.8 mmol/L; and/or |
| d. | Glycemic response: an increase in plasma glucose of >30 mg/dl (>1.7 |
| | mmol/L) after IV or intramuscular glucagon administration. |

| 4. | No requirement for inpatient management (continuous IV glucose infusion) |
|---|---|
| | for maintenance of euglycemia; |
| 5. | Has at least an average of 3 documented episodes of hypoglycemia |
| | (<70 mg/dL [<3.9 mmol/L]) per week (at least 6 episodes of hypoglycemia |
| | during the 2-week Run-In Period); |
| 6. | If female and postmenarchal, must meet the following criteria: not breast |
| | feeding or lactating, not have a positive serum pregnancy test result at |
| | screening Visit 1, agree to use appropriate birth control (failure rate of less |
| | than 1% per year when used consistently and correctly) during duration of |
| | study and for 4 weeks after the last dose of study drug, or not be of child- |
| | bearing potential (<11 years of age and premenarchal or, if >11 years of age, |
| | documented as surgically sterile). Periodic abstinence (eg, calendar, |
| | ovulation, symptothermal, or post-ovulation methods) and withdrawal are not |
| | acceptable methods of contraception. |
| 7. | Males engaged in sexual relations with a female of childbearing potential |
| | must utilize a highly effective method of contraception from the time of |
| | signing informed consent or assent as age appropriate until 74 days after the |
| | last dose of study drug. |
| Exclusion Criteria: | |
| General exclusions | |
| 1. | Receipt of an investigational or other study drug within 5 half-lives of the |
| | investigational drug and first dose administration of study drug; |
| 2. | Participation in another interventional clinical study within 30 days before a |
| | Screening Visit; Participation in studies employing investigational use of |
| | 18FDOPA (¹⁸F-dihydroxyphenylalanine) during positron emission |
| | tomography imaging is acceptable. |
| 3. | Known or suspected allergy to the study drug or related products; |
| 4. | Clinically significant active infection within 14 days before the first dose of |
| | study drug; |
| Disease-related exclusions | |
| 5. | Suspected of having a transient form of CHI (eg, transient hyperinsulinism |
| | due to maternal diabetes or perinatal stress); |
| 6. | Evidence of other potential cause of hypoglycemia (not CHI); |
| 7. | Requirement for exogenous insulin; |
| 8. | Any clinically significant abnormality, that in the opinion of the Investigator, |
| | would affect the patient's ability to participate in the study or benefit from |
| | treatment with avexitide; |
| 9. | Presence of any clinically relevant renal, hepatic, pancreatic, cardiovascular, |
| | neurological, hematological, or gastrointestinal abnormality that may |
| | preclude the patient from safely completing the study; |
| | Note: Patients with pancreatic abnormalities related to CHI and/or treatment |
| | thereof (e.g., history of pancreatectomy) are eligible. |
| 10. | Presence of any malignancy within 3 years, except for basal or squamous cell |
| | carcinoma of the skin. Patients with a history of other malignancies that have |
| | been treated with curative intent and which have no recurrence within 3 years |
| | may also be eligible if approved by the Sponsor Medical Monitor; |
| Exclusions based on laboratory or physical examination findings | |
| 11. | Documented glycosylated hemoglobin ≥6.5% within 6 months prior to a |
| | Screening Visit; |
| 12. | Abnormal liver function, defined as transaminases (alanine aminotransferase |
| | or aspartate aminotransferase) levels >2.5 × upper limit of normal (ULN) |
| | reference range, and/or total bilirubin level >2 × ULN at Screening Visit 1a; |
| | Note: Neonates with total bilirubin level <4 × ULN at Screening Visit 1a with |
| | a clinical diagnosis of physiological jaundice (within first 14 days of life) or |
| | breast-feeding jaundice are eligible if their total bilirubin levels are |
| | decreasing prior to start of study drug. |
| 13. | Abnormal renal function for the patient age, as calculated using the Schwartz |
| | formula at a Screening Visit 1; |
| 14. | Physical examination or electrocardiogram (ECG) with clinically significant |
| | abnormality, as judged by the Investigator, at Screening Visit 1a; or |
| Exclusions based on recent or concomitant medication or drug use | |
| 15. | Current use of medication(s) that affect(s) glucose metabolism other than |
| | diazoxide or somatostatin analogues. |
| | Note: Patients who are on diazoxide or a somatostatin analogue may remain |
| | on treatment, provided the dose remains stable throughout the study duration. |

| | |
|---|---|
| Primary | Effect of Avexitide on the Hypoglycemia event rate (Level 1) (EASD and |
| Outcome | ADA) as measured by self-monitoring of blood glucose (SMBG). |
| Measures | |
| Secondary | Clinically important hypoglycemia event rate (Level 2) (EASD and ADA) |
| Outcome | defined as the number of events <54 mg/dL (<3.0 mmol/L) observed per |
| Measures | week, as measured by CGM and sustained for at least 15 minutes; |
| | Clinically important hypoglycemia event rate (Level 2) (EASD and ADA) |
| | defined as the number of events <54 mg/dL (<3.0 mmol/L) observed per |
| | week, as measured by SMBG; |
| | TBR Level 2, defined as the percent time in clinically important |
| | hypoglycemia (<54 mg/dL [<3.0 mmol/L]) as measured by CGM; |
| | TIR, defined as the percent time in the glucose range of 70 to 180 mg/dl (3.9 |
| | to 10.0 mmol/L), inclusive, as measured by CGM; |
| | Total carbohydrates administered via oral route, nasogastric tube, or |
| | gastrostomy tube per week to treat or prevent hypoglycemia; |
| | Total nightly carbohydrates administered via oral route, nasogastric tube, or |
| | gastrostomy tube per week to treat or prevent hypoglycemia; and |
| | Severe hypoglycemia event rate (Level 3) (EASD and ADA), defined as the |
| | total number of severe hypoglycemia events observed per week. |
| | The exploratory efficacy endpoints of this study include the following: |
| | QoL, as measured by the Pediatric Quality of Life Inventory (PedsQL) and |
| | the PedsQL Family Impact Module or the Pediatric Quality of Life Infant |
| | Scale; and |
| | Development of ADAb. |
| | The PK endpoint of this study includes the following: |
| | The PK profile of avexitide. |

TABLE 12
Study Drug Injection 40, 100, and 200 mg/mL Single Dose Volume as a Function of Body Weight Range

| Body | Dose 0.9 mg/kg | | | Dose 1.8 mg/kg | | | Dose 2.7 mg/kg | | | Dose 3.6 mg/kg | | |
|---|---|---|---|---|---|---|---|---|---|---|---|---|
| Weight | Volume | | | Volume | | | Volume | | | Volume | | |
| Range | 40 | 100 | 200 | 40 | 100 | 200 | 40 | 100 | 200 | 40 | 100 | 200 |
| (kg) | mg/mL | mg/mL | mg/mL | mg/mL | mg/mL | mg/mL | mg/mL | mg/mL | mg/mL | mg/mL | mg/mL | mg/mL |
| 2.3-3 | 0.05 | | | 0.10 | | | 0.15 | | | 0.20 | | |
| >3-4 | 0.05 | | | 0.15 | | | 0.20 | | | 0.30 | | |
| >4-5 | 0.10 | | | 0.20 | | | 0.30 | | | 0.40 | | |
| >5-6 | 0.10 | | | 0.25 | | | 0.35 | | | 0.50 | | |
| >6-7 | 0.15 | | | 0.30 | | | 0.45 | | | | 0.25 | |
| >7-8 | 0.15 | | | 0.35 | | | 0.50 | | | | 0.30 | |
| >8-9 | 0.20 | | | 0.40 | | | | 0.25 | | | 0.30 | |
| >9-10 | 0.20 | | | 0.45 | | | | 0.25 | | | 0.35 | |
| >10-11 | 0.25 | | | 0.50 | | | | 0.30 | | | 0.40 | |
| >11-12 | | 0.10 | | | 0.20 | | | 0.30 | | | 0.40 | |
| >12-14 | | 0.10 | | | 0.25 | | | 0.35 | | | 0.45 | |
| >14-17 | | 0.15 | | | 0.30 | | | 0.40 | | | | 0.25 |
| >17-20 | | 0.15 | | | 0.35 | | | 0.45 | | | | 0.30 |
| >20-23 | | 0.20 | | | 0.40 | | | 0.50 | | | | 0.40 |
| >23-25 | | 0.20 | | | 0.45 | | | | 0.30 | | | 0.45 |
| >25-30 | | 0.25 | | | 0.50 | | | | 0.35 | | | 0.50 |
| >28-30 | | | 0.15 | | | 0.25 | | | 0.40 | | | 0.55^{a} |
| >30-35 | | | 0.15 | | | 0.30 | | | 0.45 | | | 0.60^{a} |
| >35-40 | | | 0.15 | | | 0.35 | | | 0.50 | | | 0.65^{a} |
| >40-45 | | | 0.20 | | | 0.40 | | | 0.60^{a} | | | 0.80^{a} |
| >45-50 | | | 0.20 | | | 0.45 | | | 0.65^{a} | | | 0.85^{a} |
| >50-56 | | | 0.25 | | | 0.50 | | | 0.75^{a} | | | 1.00^{a} |

^{a}Doses drawn from 2 vials.

### Example 4: Long-Term Safety and Efficacy of Avexitide in Children with Congenital Hyperinsulinism (EIG-AVX-007-CHI)

Patients who have completed treatment in either the study described in Example 2 (referred to as Study EIG-AVX-005-CHI) or the study described in Example 3 (referred to as Study EIG-AVX-006-CHI) and meet the entry criteria will be invited to enroll into this Phase 3, multicenter, long-term, open-label extension study. Treatment will start at a dose of 0.9 mg/kg BID to 3.6 mg/kg BID, with dose escalation based on efficacy and tolerability as assessed during each study visit over an up to 2 year period. Dose up-titration will occur if the patient experiences ≥1 hypoglycemia (Level 1) event per week, as recorded in the patient electronic diary (eDiary), as tolerated, and dose down-titration will occur if tolerability issues arise. Additional information regarding the trial is provided in Table 13.

During the dose titration period, patients who previously participated in Study EIG-AVX-005-CH6 will initially receive avexitide at the same total daily dose they were on at the end of the study; however, doses will be administered on a twice daily (BID) basis. During the dose titration period, patients who previously participated in Study EFG-AVX-006-CHI will initiate at a dosing regimen of 0.9 mg/kg BID and will be escalated to 1.8 mg/kg BID, then 2.7 mg/kg BID, and then to 3.6 mg/kg BID based on efficacy and as tolerated.

TABLE 13

| Eligibility | Patients must have completed treatment in either Study EIG-AVX-005-CHI or | |
|---|---|---|
| Criteria | Study EIG-AVX-006-CHI before study entry to be eligible for enrollment; | |
| | Exclusion Criteria: | |
| | 1. | Participation in another interventional clinical study other than Study EIG- |
| | | AVX-005-CHI or Study EIG-AVX-006-CHI within 30 days before |
| | | Screening. Participation in studies employing investigational use of positron |
| | | emission tomography (PET) imaging within 30 days is acceptable. |
| | 2. | Receipt of an investigational or other study drug other than avexitide within 5 |
| | | half-lives of the drug, not inclusive of receipt of ¹⁸F-DOPA (¹⁸F- |
| | | dihydroxyphenylalanine), used during PET imaging; diazoxide; or |
| | | somatostatin analogues; |
| | 3. | Requirement for exogenous insulin. |

| | |
|---|---|
| Primary | Hypoglycemia event rate (Level 1), defined as the number of events with glucose <70 |
| Outcome | mg/dL (<3.9 mmol/L) observed within a specific time interval (e.g., every |
| Measures | 2, 4, or 8 weeks as appropriate) over the avexitide treatment period, as measured |
| | by self-monitoring of blood glucose (SMBG). |
| Secondary | Clinically important hypoglycemia event rate (Level 2), defined as the |
| Outcome | number of events with glucose <54 mg/dL (<3.0 mmol/L) observed within a |
| Measures | specific time interval (eg, every 2, 4, or 8 weeks as appropriate) over the |
| | avexitide treatment period, as measured by SMBG; |
| | TIR, defined as the percent time in the glucose range of 70 to 180 mg/dl (3.9 |
| | to 10.0 mmol/L), inclusive, as measured by CGM; |
| | TBR Level 1, defined as the percent time in hypoglycemia (<70 mg/dl [<3.9 |
| | mmol/L]) as measured by CGM; |
| | TBR Level 1 Nocturnal, defined as the percent time in nocturnal |
| | hypoglycemia (<70 mg/dL [<3.9 mmol/L]; 12 AM to 8 AM) as measured by |
| | CGM; |
| | TBR Level 2, defined as the percent time in clinically important |
| | hypoglycemia (<54 mg/dL [<3.0 mmol/L]) as measured by CGM; |
| | Total and nightly carbohydrates administered via oral route, NG tube, or |
| | gastrostomy tube within a specific time interval over the avexitide treatment |
| | period; |
| | Time to removal of NG tube or gastrostomy tube; |
| | Time to pancreatic surgery (subtotal or total pancreatectomy); |
| | Diazoxide dose; |
| | Somatostatin analog dose; |
| | QoL, as measured by the Pediatric Quality of Life Inventory (PedsQL) and |
| | the PedsQL Family Impact Module, or the Pediatric Quality of Life Infant |
| | Scale; and |
| | Development of antidrug antibody (ADAb). |
| | Long-term pharmacokinetic profile of avexitide. |

According to various embodiments of this disclosure, additional information relating to avexitide and hyperinsulinism (e.g., CHI), including clinical trials, demographics of participants, diagnostic criteria, medical history of participants, and related information can be found in:
Adzick N. S., et al., Surgical treatment of congenital hyperinsulinism: Results from 500 pancreatectomies in neonates and children. J Pediatr Surg. 2019 January; 54(1):27-32.
Aguilar-Bryan L. et al., Cloning of the beta cell high-affinity sulfonylurea receptor: A regulator of insulin secretion. Science. 1995. 21; 268(5209):423-426.
Avatapalle B. et al., Drug-induced hepatitis following use of octreotide for long-term treatment of congenital hyperinsulinism. BMJ Case Rep 2012; 30: 2012.
Beal, S. L., et al., NONMEM User's Guide, Part I-VII. San Francisco: UCSF: University of California at San Francisco; 1988-1992.
Beltrand J. et al., Glucose metabolism in 105 children and adolescents after pancreatectomy for congenital hyperinsulinism. Diabetes Care. 2012 February; 35(2):198-203.
Brar P. C. et al., Management and Appropriate Use of Diazoxide in Infants and Children with Hyperinsulinism. J Clin Endocrinol Metab. 2020 Dec. 1; 105(12):dgaa543
Calabria A. C. et al., GLP-1 receptor antagonist exendin-(9-39) elevates fasting blood glucose levels in congenital hyperinsulinism owing to inactivating mutations in the ATP-sensitive K+ channel. Diabetes. 2012. 61(10):2585-2591. (ClinicalTrials.gov Identifier: NCT000571324)
Calabria A. C. et al., Postprandial hypoglycemia in children after gastric surgery: Clinical characterization and pathophysiology. Horm Res Paediatr. 2016. 85(2):140-146. Epub 23 Dec. 2015
Craig C. M. et al., Critical Role for GLP-1 in symptomatic post-bariatric hypoglycaemia. Diabetologia. 2017. 60(3):531-540.
Craig C. M. et al., Efficacy and pharmacokinetics of subcutaneous exendin (9-39) in patients with post-bariatric hypoglycemia. Diabetes, Obes, Metab. 2018.20(2):352-361.
Craig C. M. et al., PREVENT: A Randomized, Placebo-Controlled Crossover Trial of Avexitide for Treatment of Post-Bariatric Hypoglycemia. J Clin Endocrinol Metab. 2021. doi:10.1210/clinem/dgab103
De León D. D. & Stanley C. A., Mechanisms of Disease: advances in diagnosis and treatment of hyperinsulinism in neonates. Nat Clin Pract Endocrinol Metab. 2007 Jan; 3(1):57-68.
De León D. D. et al., Exendin-(9-39) corrects fasting hypoglycemia in SUR-1−/− mice by lowering cAMP in pancreatic β-cells and inhibiting insulin secretion. J Biol Chem. 2008. 283(38):25786-25793.
Edwards M. B. et al., Glucagon-like peptide 1 has a physiological role in the control of postprandial glucose in humans. Diabetes. 1999. 48:86-93.
Eng J. et al., Isolation and characterization of exendin-4, an exendin-3 analogue, from heloderma suspectum venom. Further evidence for an exendin receptor on dispersed acini from guinea pig pancreas. J Biol Chem. 1992. 267(11):7402-7405.
EMEA. Guideline on reporting the results of population pharmacokinetic analyses.2007: Available from:
http://www.ema.europa.eu/docs/en_GB/document_library/Scientific_guideline/2009/09/WC500003067.pdf.
FDA. Guidance for Industry Population Pharmacokinetics Available from: http://www.fda.gov/downloads/ScienceResearch/SpecialTopics/WomensHealthRe search/UCM133184.pdf. 1999.
Helleskov A. et al., Both Low Blood Glucose and Insufficient Treatment Confer Risk of Neurodevelopmental Impairment in Congenital Hyperinsulinism: A Multinational Cohort Study. Front Endocrinol (Lausanne). 2017 Jul. 10; 8:156.
Herrera A. et al., Prevalence of Adverse Events in Children With Congenital Hyperinsulinism Treated With Diazoxide. J Clin Endocrinol Metab. 2018 Dec. 1; 103(12):4365-4372
Jacobs D. G. et al., Growth and development in patients operated on for islet cell dysplasia. J Pediatr Surg. 1986 December; 21(12):1184-9.
Laje P. et al., Necrotizing enterocolitis in neonates receiving octreotide for the management of congenital hyperinsulinism. Pediatr Diabetes. 2010 March; 11(2):142-7
Leibowitz G. et al., Hyperinsulinemic hypoglycemia of infancy (nesidioblastosis) in clinical remission: high incidence of diabetes mellitus and persistent β-cell dysfunction at long term follow up. J Clin Endocrinol Metab 1995. 80: 386-392
Li C. et al., A signaling role of glutamine in insulin Secretion J. Biol. Chem. 2004. 279, 13393-13401.
Lord K. et al., High Risk of Diabetes and Neurobehavioral Deficits in Individuals With Surgically Treated Hyperinsulinism. J Clin Endocrinol Metab. 2015 November; 100(11):4133-9.
Ludwig A. et al., Glucose metabolism and neurological outcome in congenital hyperinsulinism. Semin Pediatr Surg. 2011 February; 20(1):45-9.
Kapoor R. R. et al., Advances in the diagnosis and management of hyperinsulinemic hypoglycemia. Nat Clin Pract Endocrinol Metab. 2009. 5(2):101-112
McMahon A. W. et al., Octreotide use and safety in infants with hyperinsulinism. Pharmacoepidemiol Drug Saf 2017; 26: 26-31.
Meissner T. et al., Long-term follow-up of 114 patients with congenital hyperinsulinism. Eur J Endocrinol. 2003 July; 149(1):43-51.
Menni F. et al., Neurologic outcomes of 90 neonates and infants with persistent hyperinsulinemic hypoglycemia. Pediatrics. 2001 March; 107(3):476-9.
Mohnike K. et al., Long-term non-surgical therapy of severe persistent congenital hyperinsulinism with glucagon. Horm Res. 2008; 70(1):59-64.
Ng C. M. et al., Population pharmacokinetics of exendin-(9-39) and clinical dose selection in patients with congenital hyperinsulinism. Br J Clin Pharmacol. 2018 March; 84(3):520-532
R Core Team. R: A language and environment for statistical computing. R Foundation for Statistical Computing, Vienna, Austria. 2020. Available at www.R-project.org.
Salehi M. et al., Gastric bypass surgery enhances glucagon-like peptide-1 stimulated postprandial insulin secretion in humans. Diabetes. 2011. 60:2308-2314.
Salehi M., et al., Blockade of glucagon-like peptide 1 receptor corrects postprandial hypoglycemia after gastric bypass, Gastroenterology. 2014. 146(3):669-680.
Schirra J. et al., Exendin (9-39) amide is an antagonist of glucagon-like peptide-1(7-36) amide in humans. J Clin Invest. 1998. 101(7):1421-1430.
Seino S. & Miki T., Physiological and pathophysiological roles of ATP-sensitive K+ channels. Prog Biophys Mol Biol. 2003 February; 81(2):133-76.
Serre V. et al., 1998. Exendin-(9-39) is an inverse agonist of the murine glucagon-like peptide-1 receptor: Implications for basal intracellular cyclic adenosine 3′,5′-monophosphate levels and β-cell glucose competence. Endocrinology 139; 4448-4454.
Stanley C. A., Perspective on the Genetics and Diagnosis of Congenital Hyperinsulinism Disorders. J Clin Endocrinol Metab. 2016 March; 101(3):815-26.
Steinkrauss L. et al., Effects of hypoglycemia on developmental outcome in children with congenital hyperinsulinism. J Pediatr Nurs. 2005 April; 20(2):109-18.
Tan M. J. et al., Safety, efficacy and pharmacokinetics of repeat subcutaneous dosing of avexitide (exendin 9-39) for treatment of post-bariatric hypoglycaemia. Diabetes Obes Metab. 2020. 8:1406-1416.
Vajravelu M. E. et al., Continuous Intragastric Dextrose: A Therapeutic Option for Refractory Hypoglycemia in Congenital Hyperinsulinism. Horm Res Paediatr. 2019; 91(1):62-68.
van der Steen I. et al., A Multicenter Experience with Long-Acting Somatostatin Analogues in Patients with CongenitalHyperinsulinism. Horm Res Paediatr, 2018; 89: 82-89.
Wald M. et al., Glucagon therapy as a possible cause of erythema necrolyticum migrans in two neonates with persistent hyperinsulinaemic hypoglycaemia. Eur J Pediatr. 2002 November; 161(11):600-3

One skilled in the art will readily appreciate that the present disclosure is well adapted to carry out the objects and obtain the ends and advantages mentioned, as well as those inherent therein. Although the foregoing invention has been described in some detail by way of illustration and example for purposes of clarity of understanding, one of skill in the art will appreciate that many modifications and variations of this invention can be made without departing from its spirit and scope, as will be apparent to those skilled in the art. The specific embodiments described herein are offered by way of example only and are not meant to be limiting in any way. It is intended that the specification and examples be considered as exemplary only, with the true scope and spirit of the invention being indicated by the following claims. Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

All publications, patents, patent applications or other documents cited herein are hereby incorporated by reference in their entirety for all purposes to the same extent as if each individual publication, patent, patent application, or other document was individually indicated to be incorporated by reference for all purposes.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

What is claimed is:

1. A method of treating a subject with congenital hyperinsulinism (CHI), wherein the subject is an infant receiving continuous intravenous glucose infusion at a GIR, the method comprising subcutaneously administering a therapeutically effective amount of avexitide to the subject.

2. A method of reducing a glucose infusion rate (GIR) for a subject having congenital hyperinsulinism (CHI), the method comprising administering a therapeutically effective amount of avexitide to the subject.

3. The method of claim 2, wherein the subject requires continuous glucose infusion.

4. The method of claim 1 or 2, wherein the subject has a baseline GIR of 1 to 50 mg/kg/min.

5. The method of claim 1, wherein the avexitide is administered subcutaneously.

6. The method of claim 1 or 5, wherein the avexitide is administered twice daily (BID) or three times daily (TID).

7. The method of claim 1 or 5, wherein the avexitide is administered at a total daily dose from 1 mg to 175 mg.

8. The method of claim 1 or 5, wherein the avexitide is administered at a total daily dose from 2.7 to 10.8 mg.

9. The method of claim 1 or 5, wherein the avexitide is administered at a dose of 0.5 to 80 mg BID or TID.

10. The method of claim 1 or 5, wherein the avexitide is administered at a dose of 0.9 to 2.7 mg/kg TID, optionally for at least 72 hours.

11. The method of claim 1 or 5, wherein the avexitide is administered at a dose of 0.9 to 3.6 mg/kg TID, optionally for at least 72 hours.

12. The method of claim 1, wherein the avexitide is administered by intravenous infusion.

13. The method of claim 12, wherein the avexitide is administered at a rate from 100-1000 pmol/kg/min.

14. The method of claim 12, wherein the avexitide is administered for at least 6 hours.

15. The method of any one of claims 1 or 5, wherein treatment is continued for at least 24 months.

16. The method of claim 1 or 2, wherein the subject is less than 2 years old.

17. The method of claim 1 or 2, wherein the subject is less than 1 year old.

18. The method of claim 1 or 2, wherein the subject is a neonate.

19. The method of claim 1 or 2, wherein the subject is less than 14 days old.

20. The method of claim 1 or 2, wherein the subject is 14 to 364 days old.

21. The method of claim 1 or 2, wherein the subject is unresponsive to medical treatment with diazoxide or octreotide.

22. The method of claim 1 or 2, wherein the subject is responsive to medical treatment with diazoxide.

23. The method of claim 1 or 2, wherein the CHI is associated with a genetic abnormality, a mutation, or a syndrome.

24. The method of claim 23, wherein the genetic abnormality, mutation, or syndrome is a mutation in a gene encoding a sulfonylurea receptor (SUR-1), a gene encoding glucokinase (GCK), a gene encoding glutamate dehydrogenase (GLUD-1), a gene encoding mitochondrial enzyme short-chain 3-hydroxyacyl-CoA dehydrogenase (HADHSC), and/or any of the genes listed in Table 1.

25. The method of claim 1 or 2, wherein the subject during hypoglycemia has at least one of hyperinsulinemia, hypofattyacidemia, hypoketonemia, or a glycemic response.

26. The method of claim 1 or 2, wherein the administering reduces the GIR required to maintain euglycemia of the subject.

27. The method of claim 1 or 2, wherein the administering reduces the GIR of the subject to less than or equal to 10 mg/kg/min.

28. The method of claim 1 or 2, wherein the administering reduces the GIR of the subject by 5% to 100%.

29. The method of claim 1 or 2, wherein the administering reduces the GIR of the subject by 15% to 60%.

30. The method of claim 1 or 2, wherein the administering reduces the GIR to 0.

31. The method of claim 1 or 2, wherein the administering permits stopping continuous glucose infusion for the subject.

32. The method of claim 1 or 2, wherein the administering reduces at least one of the total amount or volume of glucose required to be administered to the subject to maintain euglycemia or the need for IV carbohydrate rescue of the subject.

33. The method of claim 1 or 2, wherein the administering reduces at least one of hypoglycemia event rate or clinically important hypoglycemia event rate.

34. The method of claim 1 or 2, wherein the administering reduces time to central or peripheral line removal, and optionally, time to discharge readiness.

35. The method of claim 1 or 2, wherein the administering permits avoidance of performing a pancreatectomy on the subject.

36. A method of treating congenital hyperinsulinism (CHI) in a subject, the method comprising subcutaneously administering a total daily dose of 4 mg to 400 mg of avexitide to the subject.

37. The method of claim 36, wherein the subject has a body weight of 56 kg or less, optionally 2.3 to 56 kg.

38. The method of claim 37, wherein the avexitide is administered at a total daily dose from 4 mg to 210 mg.

39. The method of claim 37, wherein the avexitide is administered at 0.9 to 3.6 mg/kg BID.

40. The method of any one of claims 37 to 39, wherein the subject has been administered avexitide for at least 10 weeks, and wherein the subject is subsequently administered avexitide at a dose of 1.8 kg/mg to 3.6 mg/kg BID for at least 24 months.

41. The method of claim 36, wherein the subject has a body weight of more than 56 kg.

42. The method of claim 40, wherein the avexitide is administered at a total daily dose from 100 to 400 mg.

43. The method of claim 40, wherein the avexitide is administered at 50 mg to 200 mg BID.

44. The method of any one of claims 36 to 43, wherein the subject has been administered avexitide for at least 10 weeks, and wherein the subject is subsequently administered avexitide at a dose of 100 mg to 200 mg BID for at least 24 months.

45. The method of claim 36, wherein the avexitide is administered twice daily (BID).

46. A method of reducing the hypoglycemia event rate for a subject having congenital hyperinsulinism (CHI), the method comprising administering therapeutically effective dose of avexitide to the subject, wherein the therapeutically effective dose is a total daily dose of 4 mg to 210 mg if the subject weighs 56 kg or less, optionally 2.3 to 56 kg, and is a total daily dose of 100 mg to 400 mg if the subject weighs more than 56 kg.

47. The method of claim 46, wherein the avexitide is administered subcutaneously.

48. The method of claim 47, wherein the avexitide is administered twice daily (BID).

49. The method of claim 47, wherein the subject has a body weight of 56 kg or less, and wherein the avexitide is administered at 0.9 to 3.6 mg/kg BID.

50. The method of claim 47, wherein the subject has a body weight of more than 56 kg, and wherein the avexitide is administered at 50 mg to 200 mg BID.

51. The method of claim 36 or 47, wherein the total volume of injectate is 0.05 to 1 mL.

52. The method of claim 46, wherein the avexitide is administered by intravenous infusion.

53. The method of claim 52, wherein the avexitide is administered at a rate from 100-1000 pmol/kg/min.

54. The method of claim 52, wherein the avexitide is administered for at least 6 hours.

55. The method of any one of claims 36 or 46, wherein treatment is continued for at least 24 months.

56. The method of claim 36 or 46, wherein the subject is less than 18 years old.

57. The method of claim 36 or 46, wherein the subject is 1 year old to 12 years old.

58. The method of claim 36 or 46, wherein the subject is an infant.

59. The method of claim 36 or 46, wherein the subject is a neonatal subject.

60. The method of claim 36 or 46, wherein the subject is 18 years old or older.

61. The method of claim 36 or 46, wherein the CHI is associated with a genetic abnormality, a mutation, or a syndrome.

62. The method of claim 61, wherein the genetic abnormality, mutation, or syndrome is a mutation in a gene encoding a sulfonylurea receptor (SUR-1), a gene encoding glucokinase (GCK), a gene encoding glutamate dehydrogenase (GLUD-1), a gene encoding mitochondrial enzyme short-chain 3-hydroxyacyl-CoA dehydrogenase (HADHSC), and/or any of the genes listed in Table 1.

63. The method of claim 36 or 46, wherein the subject during hypoglycemia has at least one of hyperinsulinemia, hypofattyacidemia, hypoketonemia, or a glycemic response.

64. The method of claim 36 or 46, wherein the subject has documented uncontrolled hypoglycemia and, optionally, wherein the subject does not require continuous glucose infusion.

65. The method of claim 36 or 46, wherein treatment thereby reduces in the subject at least one of hypoglycemia event rate, clinically important hypoglycemia event rate, or severe hypoglycemia event rate.

66. The method of claim 36 or 46, wherein treatment thereby reduces in the subject at least one of TBR Level 1, TBR Level 1 Nocturnal, TBR Level 2, or TIR.

67. The method of claim 36 or 46, wherein treatment thereby permits reduction of at least one of total carbohydrates administered via oral route, nasogastric tube, or gastrostomy tube per week to treat or prevent hypoglycemia events.

68. The method of claim 36 or 46, wherein treatment thereby permits reduction of total nightly carbohydrates administered.

69. The method of claim 36 or 46, wherein treatment thereby permits removal of a nasogastric tube or gastrostomy tube from the subject.


SEQUENCE LISTING (1 sequence)

SEQ ID NO: 1 - protein, 31 aa, Artificial Sequence
  avexitide, exendin(9-39)
DLSKQMEEEAVRLFIEWLKNGGPSSGAPPPS